(19) United States
(12) Patent Application Publication
(10) Pub. No.: US 2024/0368154 A1
(43) Pub. Date: Nov. 7, 2024

(54) SELECTIVE INHIBITORS OF ROCK2 PROTEIN KINASES AND USES THEREOF

(71) Applicant: GENOSCO Inc., Billerica, MA (US)

(72) Inventors: Wongil LEE, Bedford, MA (US); William DEVINE, Woburn, MA (US); R. Bruce DIEBOLD, Waltham, MA (US); So Young HWANG, Lexington, MA (US); Sang-Ae SEUNG, Lexington, MA (US); Miyong YONG, Belmont, MA (US); Ravi DASHNAMOORTHY, Billerica, MA (US); Anu GUPTA, Billerica, MA (US); Jaekyoo LEE, North Andover, MA (US); Jong Sung KOH, Cambridge, MA (US)

(21) Appl. No.: 18/601,424

(22) Filed: Mar. 11, 2024

Related U.S. Application Data
(60) Provisional application No. 63/496,815, filed on Apr. 18, 2023.

(51) Int. Cl. C07D 471/04; A61K 31/4155; A61K 31/416; A61K 31/437; A61K 31/519; C07D 405/14; C07D 487/04

(52) U.S. Cl. CPC C07D 471/04; A61K 31/4155; A61K 31/416; A61K 31/437; A61K 31/519; C07D 405/14; C07D 487/04

(57) ABSTRACT

The present invention relates to novel substituted bicyclic derivatives that can selectively inhibit ROCK2 and/or Rho-kinase mediated phosphorylation of myosin light chain phosphates, compositions comprising the derivatives, methods for preparing the derivatives and/or compositions, and methods for using the derivatives and/or compositions.

### CROSS-REFERENCE TO RELATED APPLICATION(S)

This application claims priority from U.S. Provisional Application No. 63/496,815 filed Apr. 18, 2023, which is incorporated herein by reference in its entirety.

### TECHNICAL FIELD

The present invention relates to the novel substituted bicyclic derivatives that can selectively inhibit ROCK2 and/or Rho-kinase mediated phosphorylation of myosin light chain phosphates, the process for their preparation, and formulation thereof and their use for preparing a medicament, as well as pharmacologically acceptable salts, diastereomers, enantiomers, racemates.

### BACKGROUND

The Rho-associated coiled-coil containing protein kinases (ROCKs/Rho-kinase/Rho-associated kinase) are downstream effectors of the small GTPase Rho (Rho A, Rho B, Rho C, and Rho E) and belong to the family of serine/threonine kinases. The active GTP bound form of Rho mediates several biological functions through the action of ROCKs, including smooth muscle contractions, cell motility, and cytokinesis. The ROCK proteins were identified in 1996 as proteins that bind to Rho GTPase. Two proteins were independently isolated as p160 and p164. Later, these were recognized as ROCK-1 and ROCK-2, respectively and as isoforms of the Rho-associated kinase. The two isoforms, ROCK-1 (ROCK-b/p160) and ROCK-2 (ROCK-a/p164), share 92% similarity in their amino acid sequence. Their structure comprises an N-terminally located catalytic kinase domain, followed by a coiled-coil containing region (600 amino acids) with a Rho-binding domain and a pleckstrin homology (PH) domain at the C terminus. They have varying locations, and distinct physiological roles have been identified for each. The ROCK-1 transcript (gene located on chromosome 18) is ubiquitous, with more prominent expression in liver, kidney, spleen, testis, thymus, and blood corpuscles, whereas ROCK-2 mRNA (chromosome 2) is expressed more abundantly in skeletal muscles and brain, suggesting that they have specialized roles in these locations.

ROCK inhibitors have been considered for use in numerous diseases, such as cerebral ischemia, hypertension, erectile dysfunction, glaucoma, osteoporosis, cardiac hypertrophy, diabetic cardiomyopathy, retinopathy, pulmonary hypertension, and atherosclerosis. However, their implementation is limited because of a lack of knowledge regarding the involvement of the particular ROCK isoform. Whether isoform-specific targeting or combined ROCK inhibition would provide a better therapeutic outcome is yet to be verified. Despite their incomplete specificity towards the ROCK isoforms as well as other serine/threonine kinases, such as PRK2, PKC, cAMP-dependent protein kinase, and citron kinase, some nonspecific ROCK2 inhibitors have shown promising results in certain pathological states, such as glaucoma and hypertension. However, further work is required for isoform-selective ROCK2 inhibitors to be of clinical use, although several research-based studies have helped resolve much ambiguity over ROCK-1 and ROCK-2-specific functions. Since ROCKs play a central role in the organization of the actin cytoskeleton, it might be anticipated that the complete inhibition of both isoforms could cause adverse events in patients. Therefore, selective ROCK2 inhibition could have a greater specific therapeutic index over the dual inhibition of ROCK1 and ROCK2. Thus, understanding the discreet functions of each isoform in a particular disorder could help improve safety and specificity and expand the therapeutic application of ROCK inhibitors.

### SUMMARY

In one aspect, the present invention provides novel chemical compounds represented by Formula (1) below, which is capable of inhibiting Rho-associated coiled-coil forming protein serine/threonine kinases (ROCKs):

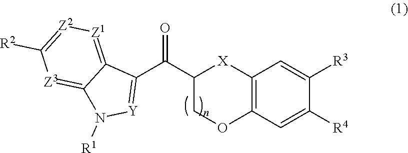

wherein X, Y, Z¹, Z², Z³, R¹, R² R³ and R⁴ are defined in the detailed description of the invention below.

In another aspect, the present invention provides pharmaceutically acceptable salts, diastereomers, enantiomers, racemates, hydrates, solvates, prodrugs of the novel compounds.

Also, the present invention provides pharmaceutically acceptable salts, hydrates, or solvates of the diastereomers, enantiomers, or racemates.

In still another aspect, the present invention provides pharmaceutical compositions each comprising the compound, salt, diastereomer, enantiomer, racemate, hydrate, solvate, prodrug, or a combination thereof.

In yet another aspect, the present invention provides methods of treating or alleviating certain ROCK-mediated diseases or disorders by using the compounds, salts, diastereomers, enantiomers, racemates, hydrates, solvates, prodrugs, or compositions. Non-limiting examples of the diseases or disorders include cardiovascular, pulmonary, inflammatory, neurological, or proliferative diseases or disorders.

In a further aspect, the present invention provides methods of preparing the compounds, salts, diastereomers, enantiomers, racemates, hydrates, solvates, and prodrugs.

### DETAILED DESCRIPTION

### 1. Compounds

In one aspect, the present invention provides a compound represented by Formula (1), a pharmaceutically acceptable salt, diastereomer, enantiomer, racemate, hydrate, solvate, or prodrug thereof:

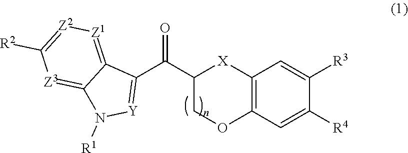

wherein:
X is CH₂ or O;
Y is N or CH;
Z¹, Z² and Z³ each are independently N or CH,
n is 0, 1, 2 or 3;
R¹ is H, C₂-C₆alkyl, or CH₂C₃-C₈cyclocloalkyl wherein the C₂-C₆alkyl or CH₂C₃-C₈cyclocloalkyl can be optionally substituted with one or more suitable substituents, for example, C₁-C₃alkyl, hydroxyl, or C₁-C₃alkoxy;
R² is 5-6 membered heteroaryl or bicyclic heteroaryl, wherein the heteroaryl or bicyclic heteroaryl can be optionally substituted with one or more suitable substituents, for example, halogen, CN, CHF₂, CF₃, C₁-C₃alkyl, or amino, wherein the 5-6 membered heteroaryl or bicyclic heteroaryl has 1-3 heteroatoms selected from the group consisting of oxygen, nitrogen and a combination thereof; and
R³ and R⁴ each are independently H, Cl, F, OH, OCD³, C₁-C₃alkyl, or C₁-C₃alkoxy, wherein the C₁-C₃alkyl or C₁-C₃alkoxy can be optionally substituted with one or more suitable substituents, for example, OH or OMe, OEt, or OPrⁱ.

In some embodiments, R¹ may be H, C₂-C₆alkyl, or C₃-C₈cyclocloalkyl wherein the C₂-C₆alkyl and C₃-C₈cyclocloalkyl can be optionally substituted with one or more suitable substituents, for example, C₁-C₃alkyl, hydroxyl, or C₁-C₃alkoxy.

In some embodiments, R² may be one of the following group:

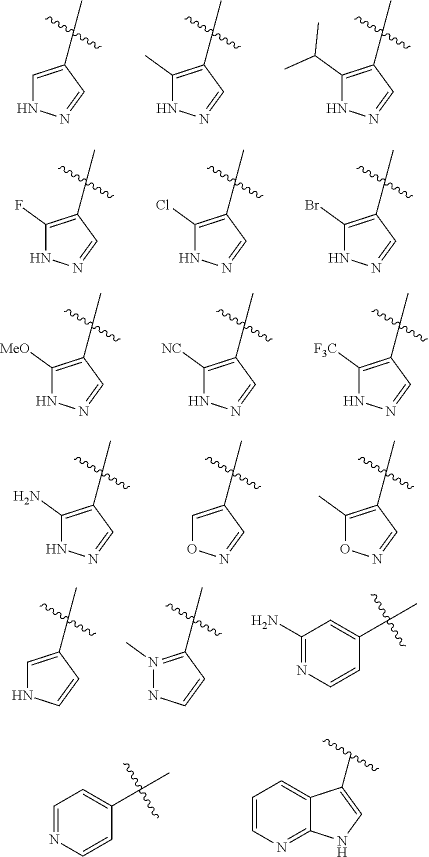

In some embodiments, R³ or R⁴ may be independently H, Cl, F, OH, methoxy, CD³, ethoxy, or iso-propoxy.

The articles “a” and “an” are used in this disclosure to refer to one or more than one (i.e., to at least one) of the grammatical objects of the article. By way of example, “an element” means one element or more than one element.

The term “and/or” is used in this disclosure to mean either “and” or “or” unless indicated otherwise. The use of the term “or” is used to mean “and/or” unless explicitly indicated to refer to alternatives only or the alternative are mutually exclusive, although the disclosure supports a definition that refers to only alternatives and “and/or.”

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment, an optionally substituted group has 2 substituents. In another embodiment, an optionally substituted group has 3 substituents. In another embodiment, an optionally substituted group has 4 substituents. In another embodiment, an optionally substituted group has 5 substituents. For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bonded to a halogen atom, a hydroxyl group, or any other substituent described herein. Thus, the term “optionally substituted” means that a given chemical moiety has the potential to contain other functional groups, but does not necessarily have any further functional groups.

The term “alkyl,” used alone or as part of a larger moiety such as “arylalkyl” or “cycloalkyl” refers to a straight or branched hydrocarbon radical having from 1 to 10 carbon atoms, or from 1-8 carbon atoms, or from 1-6 carbon atoms, or from 1-4 carbon atoms (unless stated otherwise) and includes, for example, methyl, ethyl, n-propyl, iso-propyl, n-butyl, sec-butyl, isobutyl, tert-butyl, n-pentyl, iso-pentyl, n-hexyl and the like. An alkyl can be unsubstituted or substituted with one or more suitable substituents.

The term “cycloalkyl” refers to a monocyclic or polycyclic hydrocarbon ring group having from 3 to 10 carbon atoms, or from 3 to 7 carbon atoms, in the hydrocarbon ring (unless stated otherwise) and includes, for example, cyclopropyl, cycloheptyl, cyclooctyl, cyclodecyl, cyclobutyl, adamantyl, norpinanyl, decalinyl, norbornyl, cyclohexyl, cyclopentyl, and the like. A cycloalkyl group can be unsubstituted or substituted with one or more suitable substituents.

The term “hetero” refers to the replacement of at least one carbon atom member in a ring system with at least one heteroatom such as nitrogen, sulfur, sulfoxide, sulfone, and oxygen.

As used herein, the term “halo” includes fluoro, chloro, bromo, and iodo.

As used herein, the term “alkoxy” refers to the alkyl groups above bound through oxygen, examples of which include methoxy, ethoxy, iso-propoxy, tert-butoxy, and the like. In addition, alkoxy also refers to polyethers such as —O—(CH₂)₂O—CH₃, and the like. An alkoxy can be unsubstituted or substituted with one or more suitable substituents.

As used herein, the term “aryl” refers to unsubstituted or substituted aromatic monocyclic or polycyclic groups and includes, for example, carbocyclic aromatic groups such as phenyl, naphthyl and the like, as well as heteroaromatic groups such as pyridyl, furanyl, thiophenyl, and the like. The term “aryl” also includes an aromatic ring (such as a phenyl or pyridyl ring) fused to a non-aromatic carbocyclic or heterocyclic ring. The term “aryl” may be interchangeably used with “aryl ring,” aromatic group,” and “aromatic ring.” Heteroaryl groups have 4 to 14 atoms in the heteroaromatic ring(s), 1 to 9 of which are independently selected from the group consisting of oxygen, sulfur and nitrogen. Heteroaryl groups have 1-3 heteroatoms in a 5-8 membered aromatic group. An aryl or heteroaryl can be a mono- or bicyclic aromatic group. Typical aryl and heteroaryl groups include, for example, phenyl, quinolinyl, indazoyl, indolyl, dihydrobenzodioxynyl, 3-chlorophenyl, 2,6-dibromophenyl, pyridyl, pyrimidinyl, 3-methylpyridyl, benzothienyl, 2,4,6-tribromophenyl, 4-ethylbenzothienyl, furanyl, 3,4-diethylfuranyl, naphthyl, 4,7-dichloronaphthyl, pyrrole, pyrazole, imidazole, thiazole, and the like. An aryl or heteroaryl can be unsubstituted or substituted with one or more suitable substituents.

As used herein, the term “hydroxyl” or “hydroxy” refers to —OH.

As used herein, the term “amino” refers to —NH₂.

As used herein, the term “hydroxyalkyl” refers to any hydroxyl derivative of alkyl radical. The term “hydroxyalkyl” includes any alkyl radical having one or more hydrogen atoms replaced by a hydroxy group.

As used herein, the term “arylalkyl” includes any alkyl radical having one or more hydrogen atoms replaced by an aryl group, e.g., a benzyl group, a phenethyl group, and the like.

A “substituent,” as used herein, refers to a molecular moiety that is covalently bonded to an atom within a molecule of interest. For example, a ring substituent may be a moiety such as a halogen, alkyl group, haloalkyl group or other group that is covalently bonded to an atom (preferably a carbon or nitrogen atom) that is a ring member. Substituents of aromatic groups are generally covalently bonded to a ring carbon atom. The term “substitution” refers to replacing a hydrogen atom in a molecular structure with a substituent, such that the valence on the designated atom is not exceeded, and such that a chemically stable compound (i.e., a compound that can be isolated, characterized, and tested for biological activity) results from the substitution.

As described above, certain groups can be unsubstituted or substituted with one or more suitable substituents by other than hydrogen at one or more available positions, typically 1, 2, 3, 4 or 5 positions, by one or more suitable groups (which may be the same or different). Certain groups, when substituted, are substituted with 1, 2, 3 or 4 independently selected substituents. Suitable substituents include halo, alkyl, haloalkyl, aryl, hydroxy, alkoxy, hydroxyalkyl, amino, and the like.

As used herein, the term “pharmaceutically acceptable” refers to a material, such as a carrier or diluent, which does not abrogate the biological activity or properties of the compounds described herein. Such materials are administered to an individual without causing undesirable biological effects or interacting in a deleterious manner with any of the components of the composition in which it is contained.

As used herein, the term “pharmaceutically acceptable salt” refers to a formulation of a compound that does not cause significant irritation to an organism to which it is administered and does not abrogate the biological activity and properties of the compounds described herein.

Pharmaceutically acceptable salt forms include pharmaceutically acceptable acidic/anionic or basic/cationic salts (UK Journal of Pharmaceutical and Biosciences Vol. 2(4), 01-04, 2014, which is incorporated herein by reference). Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include sodium, potassium, calcium, magnesium, diethanolamine, N-methyl-D-glucamine, L-lysine, L-arginine, ammonium, ethanolamine, piperazine and triethanolamine salts.

A pharmaceutically acceptable acid salt is formed by reaction of the free base form of a compound of Formula 1 with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid. A pharmaceutically acceptable acid addition salt of a compound of Formula 1 can comprise or be, for example, a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g., 2-naphthalenesulfonate) or hexanoate salt.

The free acid or free base forms of the compounds of the invention may be prepared by methods known to those of ordinary skill in the art (e.g., for further details see L. D. Bigley, S. M. Berg, D. C. Monkhouse, in “Encyclopedia of Pharmaceutical Technology”. Eds, J. Swarbrick and J. C. Boylam, Vol 13, Marcel Dekker, Inc., 1995, pp. 453-499; the entire teachings of which are incorporated herein by reference) from the corresponding base addition salt or acid addition salt form, respectively. For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Prodrug derivatives of the compounds of the invention may be prepared by methods known to those of ordinary skill in the art (e.g., for further details see Saulnier et al., Bioorg. Med. Chem. Letters, 1994, 4, 1985; Daniela Hartmann Jornada at. Al., Molecules 2016, 21, 42; the entire teachings of which are incorporated herein by reference). Protected derivatives of the compounds of the invention may be prepared by means known to those of ordinary skill in the art. A detailed description of techniques applicable to the creation of protecting groups and their removal can be found in T. W. Greene, “Green's Protective Groups in Organic Chemistry,” 4th edition, John Wiley and Sons, Inc., 2006, the entire teachings of which are incorporated herein by reference.

Compounds of the invention may be prepared as their individual stereoisomers by reaction of a racemic mixture of the compound with an optically active resolving agent to form a pair of diastereoisomeric compounds, separating the diastereomers and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet and Samuel H. Wilen, “Enantiomers, Racemates and Resolutions,” John Wiley And Sons, Inc., 1981, the entire teachings of which are incorporated herein by reference.

As used herein, the term “solvate” refers to a complex of variable stoichiometry formed by a solute (in this invention, a compound of Formula 1 or a pharmaceutically acceptable salt thereof) and a solvent. Such solvents for the purpose of the invention may not interfere with the biological activity of the solute. Non-limiting examples of suitable solvents include water, acetone, methanol, ethanol and acetic acid. Preferably the solvent used is a pharmaceutically acceptable solvent. Non-limiting examples of suitable pharmaceutically acceptable solvents include water, ethanol and acetic acid.

### 2. Compositions

In another aspect, the present invention provides a pharmaceutical composition comprising the compound, salt, diastereomer, enantiomer, racemate, hydrate, solvate, prodrug, or a pharmaceutical combination thereof. The composition may further comprise an additional component. A non-limiting example of the additional component includes a pharmaceutically acceptable carrier, diluent, excipient, and a combination thereof.

As used herein, the term “pharmaceutical composition” refers to a mixture of a compound described herein with other chemical components, such as carriers, stabilizers, diluents, dispersing agents, suspending agents, thickening agents, and/or excipients.

As used herein, the term “pharmaceutical combination” means a product that results from the mixing or combining of more than one active ingredient.

As used herein, the term “acceptable” with respect to a formulation, composition, or ingredient, as used herein, means having no persistent detrimental effect on the general health of the subject being treated.

As used herein, the term “carrier” refers to chemical compounds or agents that facilitate the incorporation of a compound described herein into cells or tissues.

As used herein, the term “diluent” refers to chemical compounds that are used to dilute a compound described herein prior to delivery. Diluents can also be used to stabilize compounds described herein.

Suitable pharmaceutically acceptable carriers, diluents, adjuvants, or excipients for use in the pharmaceutical compositions of the invention include tablets (coated tablets) made of for example collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another) and aerosols (propellant-containing or -free inhale solutions).

Excipients which may be used include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone) and lubricants (e.g., magnesium stearate, talc, stearic acid and sodium lauryl sulphate).

### 3. Methods of Using Compounds or Compositions

In another aspect, the present invention provides methods of treating or alleviating certain protein kinase-mediated diseases or conditions by administering a subject or patient a therapeutically effective amount of the compound, salt, diastereomer, enantiomer, racemate, hydrate, solvate, prodrug, or composition. In some embodiments, the present invention provides methods of treating or alleviating certain ROCK-mediated diseases or disorders by administering a subject or patient a therapeutically effective amount of the compound, salt, diastereomer, enantiomer, racemate, hydrate, solvate, prodrug, or composition. In some embodiments, the present invention provides methods of treating or alleviating a disease or disorder in which ROCK is known to play a role.

In a further aspect, the present invention provides methods of inhibiting enzyme activity, particularly ROCK1, ROCK2, PKCδ, PKCθ, PRK1, GSK3, PRK2, NEK1 and NEK4 kinase activity, by administering a subject or patient a therapeutically effective amount of the compound, salt, diastereomer, enantiomer, racemate, hydrate, solvate, prodrug, or composition.

In yet another aspect, the present invention provides methods of inhibiting protein kinase activity, such as, for example, ROCK kinase activity, in a biological sample by contacting the biological sample with the compound, salt, diastereomer, enantiomer, racemate, hydrate, solvate, prodrug, or composition.

As used herein, the term “inhibitor” refers to a compound which inhibits one or more kinases described herein. For example, the term “ROCK inhibitor” refers to a compound which inhibits the ROCK receptor or reduces the signaling effect.

As used herein, the term “protein kinase-mediated disease” or a “disorder or disease or condition mediated by inappropriate protein kinase activity” refers to any disease state mediated or modulated by protein kinases described herein. Such disease states include, but are not limited to, fibrotic disorders; pulmonary fibrosis including cystic and idiopathic pulmonary fibrosis, radiation induced lung injury, liver fibrosis including cirrhosis, cardiac fibrosis including arterial fibrosis, endomyocardial fibrosis, old myocardial infraction, arterial stiffness, atherosclerosis, restenosis, arthrofibrosis, Crohn's disease, myelofibrosis, Peyronie's diseases, nephrogenic systemic fibrosis, progressive massive fibrosis, retroperitoneal cavity fibrosis, scleroderma/systemic sclerosis, mediastinal fibrosis, Keloids and hypertrophic scars, glial scaring, or renal fibrosis; cardiovascular disease or disorder, such as, for example, cerebral vasospasm, hypertension, atherosclerosis, angina, myocardial infarction, ischemic/reperfusion injury, stroke, bronchial asthma; glaucoma, pre-term labor, erectile dysfunction, or renal disease, such as, for example, chronic renal failure, chronic nephritis, diabetic nephropathy, and IgA nephropathia; and proliferative disorders, such as, for example, retinopathy, fibrosis, or invasive/metastatic cancers. Such cancers include adenocarcinoma, adrenocortical cancer; bladder cancer; bone cancer; brain cancer; breast cancer; cancer of the buccal cavity; cervical cancer; colon cancer; colorectal cancer; endometrial or uterine carcinoma; epidermoid carcinoma; esophageal cancer; eye cancer; follicular carcinoma; gallbladder cancer; gastrointestinal cancer; cancer of the genitourinary tract; glioblastoma; hairy cell carcinoma; head and neck cancer; hepatic carcinoma; hepatocellular cancer; Hodgkin's disease; keratoacanthoma; kidney cancer; large cell carcinoma; cancer of the large intestine; laryngeal cancer; liver cancer; lung cancer, such as, for example, adenocarcinoma of the lung, small-cell lung cancer, squamous carcinoma of the lung, non-small cell lung cancer; melanoma; myeloproliferative disorders, neuroblastoma; ovarian cancer; papillary carcinoma; pancreatic cancer; cancer of the peritoneum; prostate cancer; rectal cancer; salivary gland carcinoma; sarcoma; squamous cell cancer; small cell carcinoma; cancer of the small intestine; stomach cancer; testicular cancer; thyroid cancer; and vulval cancer. In particular embodiments, the treated cancer is melanoma, breast cancer, colon cancer, or pancreatic cancer.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes and the like.

As used herein, the term “administration” or “administering” of the subject compound refers to providing a compound of the invention and/or prodrugs thereof to a subject in need of treatment.

As used herein, the term “effective amount” or “therapeutically effective amount” refer to a sufficient amount of a compound described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. By way of example only, a therapeutically effective amount of a compound of the invention may be in the range of e.g., about 0.01 mg/kg/day to about 1000 mg/kg/day, from about 0.1 mg/kg/day to about 500 mg/kg/day, from about 0.1 mg (×2)/kg/day to about 500 mg (×2)/kg/day.

The compounds of the present invention were screened against a kinase panel and inhibited the activity of at least one kinase on the panel. Examples of kinases include, but not limited to, ROCK1 and ROCK2.

The compounds described herein are inhibitors of ROCK kinase activity and have therapeutic benefit in the treatment of disorders associated with inappropriate kinase activity, in particular in the treatment and prevention of disease states mediated by kinases, including ROCK kinase. Therefore, the present invention provides methods of regulating and, in particular, inhibiting signal transduction cascades in which a kinase plays a role. The method generally involves administering to a subject or contacting a cell expressing the kinase with an effective amount of a compound described herein, a salt, a diastereomer, an enantiomer, a racemate, a hydrate, a solvate, a prodrug, and/or a composition thereof, to regulate or inhibit the signal transduction cascade. The methods are also used to regulate and, in particular, inhibit downstream processes or cellular responses elicited by activation of the particular kinase signal transduction cascade. The methods are also practiced in in vitro contexts or in in vivo contexts as a therapeutic approach towards the treatment or prevention of diseases characterized by, caused by, or associated with activation of the kinase-dependent signal transduction cascade.

For the therapeutic uses of compounds provided herein, including compounds of Formula 1, salts, diastereomers, enantiomers, racemates, hydrates, solvates, or prodrugs thereof, such compounds are administered in therapeutically effective amounts either alone or as part of a pharmaceutical composition. Accordingly, provided herein are pharmaceutical compositions, which comprise at least one compound provided herein, including at least one compound of Formula 1, a pharmaceutically acceptable salt, a diastereomer, an enantiomer, a racemate, a hydrate, a solvate, or prodrug thereof, and one or more pharmaceutically acceptable carriers, diluents, adjuvant or excipients.

In addition, such compounds and compositions are administered singly or in combination with one or more additional therapeutic agents. Non-limiting examples of the additional therapeutic agents may include immune checkpoint inhibitors and immunogenic cell death (ICD)-inducing chemotherapeutic agents. Non-limiting examples of the immune checkpoint inhibitor may include PD-1 inhibitors, PD-L1 inhibitors, and CTLA-4 inhibitors. Non-limiting examples of the immunogenic cell death (ICD)-inducing chemotherapeutic agent may include doxorubicin, idarubicin, mitoxantrone, tautomycin, calyculin A, salubrinal, oxaliplatin, bleomycin, and cyclophosphamide. The methods of administration of such compounds and compositions include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration. Compounds provided herein are administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such pharmaceutical compositions are formulated as tablets, pills, capsules, a liquid, an inhalant, a nasal spray solution, a suppository, a solution, a gel, an emulsion, an ointment, eye drops or ear drops.

The therapeutically effective amount will vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

The compounds of Formula 1 are useful for inhibiting one or more protein kinases and for treating diseases and disorders that are mediated by the protein kinases, such as cancer, autoimmune diseases, fibrotic disorders, cardiovascular disease, and neurodegenerative diseases.

The term “biological sample,” as used herein, means a sample outside an animal and includes, without limitation, cell cultures or extracts thereof; biopsied material obtained from an animal or extracts thereof; and blood, saliva, urine, feces, semen, tears, or other body fluids or extracts thereof Inhibition of kinase activity, particularly ROCK kinase activity, in a biological sample is useful for a variety of purposes known to one of skill in the art. Examples of such purposes include, but are not limited to, biological specimen storage and biological assays. The term “ROCK-mediated disease” or “condition,” as used herein, means any disease or other deleterious condition in which ROCK is known to play a role. ROCK participates in a variety of important physiological functions in the vasculature, including smooth muscle contraction; cell proliferation, such as, for example, vascular smooth muscle cell proliferation; and cell adhesion and migration (see Hu & Lee, Expert Opin. Ther. Targets, 9(4):715-36, 2005; Shimokawa & Takeshita, Arterioscler. Thromb. Vase. Biol. 25(9):1767-75, 2005). ROCK participates in inflammatory responses due to leukocyte migration, such as, for example, autoimmune disease and allergic reactions (see Wettschureck et al., J. Mol. Med. 80:629-38, 2002). Abnormal activation of the Rho/ROCK pathway has been observed in various disorders of the central nervous system (see Mueller et al, Nature Rev., 4:387-98, 2005). In addition, ROCK has been implicated in tumor cell migration and invasion (Riento & Ridley, Nature Rev. 4:446-56, 2004) and in osteoporosis (Ohnaka et al., Biochem. Biophys., Res. Commun. 287(2):337-4, 2001).

Specifically, the present invention relates to a method of treating or lessening the severity of a cardiovascular disease or disorder, such as, for example, cerebral vasospasm, hypertension, atherosclerosis, angina, myocardial infarction, ischemic/reperfusion injury, stroke, bronchial asthma; glaucoma, pre-term labor, erectile dysfunction, or renal disease, such as, for example, chronic renal failure, chronic nephritis, diabetic nephropathy, and IgA nephropathia; and proliferative disorders, such as, for example, retinopathy, fibrosis, or invasive/metastatic cancers. Such cancers include adenocarcinoma, adrenocortical cancer; bladder cancer; bone cancer; brain cancer; breast cancer; cancer of the buccal cavity; cervical cancer; colon cancer; colorectal cancer; endometrial or uterine carcinoma; epidermoid carcinoma; esophageal cancer; eye cancer; follicular carcinoma; gallbladder cancer; gastrointestinal cancer; cancer of the genitourinary tract; glioblastoma; hairy cell carcinoma; head and neck cancer; hepatic carcinoma; hepatocellular cancer; Hodgkin's disease; keratoacanthoma; kidney cancer; large cell carcinoma; cancer of the large intestine; laryngeal cancer; liver cancer; lung cancer, such as, for example, adenocarcinoma of the lung, small-cell lung cancer, squamous carcinoma of the lung, non-small cell lung cancer; melanoma; myeloproliferative disorders, neuroblastoma; ovarian cancer; papillary carcinoma; pancreatic cancer; cancer of the peritoneum; prostate cancer; rectal cancer; salivary gland carcinoma; sarcoma; squamous cell cancer; small cell carcinoma; cancer of the small intestine; stomach cancer; testicular cancer; thyroid cancer; and vulval cancer. In particular embodiments, the treated cancer is melanoma, breast cancer, colon cancer, or pancreatic cancer.

In another aspect, the invention provides a method of treating a fibrotic disorder in a subject comprising administering to the subject a therapeutically effective amount of a compound of Formula I. Non-limiting examples of fibrotic disorders are pulmonary fibrosis including cystic and idiopathic pulmonary fibrosis, radiation induced lung injury, liver fibrosis including cirrhosis, cardiac fibrosis including arterial fibrosis, endomyocardial fibrosis, old myocardial infraction, arterial stiffness, atherosclerosis, restenosis, arthrofibrosis, Crohn's disease, myelofibrosis, Peyronie's diseases, nephrogenic systemic fibrosis, progressive massive fibrosis, retroperitoneal cavity fibrosis, schleroderma/systemic sclerosis, mediastinal fibrosis, Keloids and hypertrophic scars, glial scaring, or renal fibrosis.

In one aspect, the present invention provides methods for treating a cell-proliferative disease or condition, such as cancer, comprising administering to a subject in need of such treatment a therapeutically effective amount of the compound of Formula 1, a pharmaceutically acceptable salt, a diastereomer, an enantiomer, a racemate, a hydrate, a solvate, a prodrug thereof, or a pharmaceutical composition or medicament thereof, wherein the cell proliferative disease or condition include, for example, lymphoma, osteosarcoma, melanoma, breast cancer, renal cancer, prostate cancer, colorectal cancer, thyroid cancer, ovarian cancer, pancreatic cancer, neuronal cancer, lung cancer, uterine cancer or gastrointestinal cancer. In one aspect, the present invention provides methods of inhibiting growth of cancer cells with the compound described herein, salt, diastereomer, enantiomer, racemate, hydrate, solvate, prodrug, or any combination thereof, or the composition described herein.

In certain embodiments, protein kinase-mediated diseases or conditions are inflammatory diseases or conditions, respiratory diseases or autoimmune diseases or conditions, such as asthma, chronic obstructive pulmonary disease (COPD), adult respiratory distress syndrome (ARDS), ulcerative colitis, Crohn's disease, bronchitis, dermatitis, allergic rhinitis, psoriasis, scleroderma, urticaria, rheumatoid arthritis, multiple sclerosis, cancer, breast cancer, HIV associated diseases or lupus.

In another aspect, the present invention provides methods for treating a cardiovascular disease by administering to a subject a therapeutically effective amount of the compound described herein, salt, diastereomer, enantiomer, racemate, hydrate, solvate, prodrug, or any combination thereof, or the composition described herein. Such a cardiovascular disease affects the heart or blood vessels and includes, for example, atherosclerosis, arrhythmia, angina, myocardial ischemia, myocardial infarction, cardiac or vascular aneurysm, vasculitis, stroke, peripheral obstructive arteriopathy of a limb, an organ, or a tissue, reperfusion injury following ischemia of an organ or a tissue, endotoxic, surgical, or traumatic shock, hypertension, valvular heart disease, heart failure, abnormal blood pressure, vasoconstriction, vascular abnormality, or inflammation.

In another aspect, the present invention provides methods of treating cancer comprising administering to a subject in need a composition comprising a therapeutically effective amount of at least one of the compounds described herein, salts, diastereomers, enantiomers, racemates, hydrates, solvates, or prodrugs thereof and a therapeutically effective amount of at least one immune checkpoint inhibitor, wherein the cancer is adenocarcinoma, adrenocortical cancer, bladder cancer, bone cancer, brain cancer, breast cancer, buccal cavity cancer, cervical cancer, colon cancer, colorectal cancer, endometrial or uterine carcinoma, epidermoid carcinoma, esophageal cancer, eye cancer, follicular carcinoma, gallbladder cancer, gastrointestinal cancer, genitourinary tract cancer, glioblastoma, hairy cell carcinoma, head and neck cancer, hepatic carcinoma, hepatocellular cancer, Hodgkin's disease, keratoacanthoma, kidney cancer, large cell carcinoma, large intestine cancer, laryngeal cancer, liver cancer, lung adenocarcinoma, small-cell lung cancer, lung squamous carcinoma, non-small cell lung cancer, melanoma, a myeloproliferative disorder, neuroblastoma, ovarian cancer, papillary carcinoma, pancreatic cancer, peritoneal cancer, prostate cancer, rectal cancer, salivary gland carcinoma, sarcoma, squamous cell cancer, small cell carcinoma, small intestine cancer, stomach cancer, testicular cancer, thyroid cancer, vulvar cancer, or any combination thereof. Examples of the checkpoint inhibitor include, not being limited to, a PD-1 inhibitor, a PD-L1 inhibitor, and a CTLA-4 inhibitor.

In another aspect, the present invention provides methods of treating a cancer comprising administering to a subject in need a composition comprising a therapeutically effective amount of at least one of the compounds described herein, salts, diastereomers, enantiomers, racemates, hydrates, solvates, or prodrugs thereof and a therapeutically effective amount of at least one immunogenic cell death (ICD)-inducing chemotherapeutic, wherein the cancer is adenocarcinoma, adrenocortical cancer, bladder cancer, bone cancer, brain cancer, breast cancer, buccal cavity cancer, cervical cancer, colon cancer, colorectal cancer, endometrial or uterine carcinoma, epidermoid carcinoma, esophageal cancer, eye cancer, follicular carcinoma, gallbladder cancer, gastrointestinal cancer, genitourinary tract cancer, glioblastoma, hairy cell carcinoma, head and neck cancer, hepatic carcinoma, hepatocellular cancer, Hodgkin's disease, keratoacanthoma, kidney cancer, large cell carcinoma, large intestine cancer, laryngeal cancer, liver cancer, lung adenocarcinoma, small-cell lung cancer, lung squamous carcinoma, non-small cell lung cancer, melanoma, a myeloproliferative disorder, neuroblastoma, ovarian cancer, papillary carcinoma, pancreatic cancer, peritoneal cancer, prostate cancer, rectal cancer, salivary gland carcinoma, sarcoma, squamous cell cancer, small cell carcinoma, small intestine cancer, stomach cancer, testicular cancer, thyroid cancer, vulvar cancer, or any combination thereof. Examples of the immunogenic cell death (ICD)-inducing chemotherapeutic include, not being limited to, doxorubicin, idarubicin, mitoxantrone, tautomycin, calyculin A, salubrinal, oxaliplatin, bleomycin, and cyclophosphamide.

In the above methods for using the compound of the invention, the compound described herein, salt, diastereomer, enantiomer, racemate, hydrate, solvate, or prodrug thereof is administered to a system comprising cells or tissues. In certain embodiments, the compound described herein, a salt, a diastereomer, an enantiomer, a racemate, a hydrate, a solvate, a prodrug, or any combination thereof is administered to a human or animal subject. In still certain embodiments, a pharmaceutical composition or a medicament comprising at least one of the compound, the salt, the diastereomer, the enantiomer, the racemate, the hydrate, the solvate, the prodrug, or any combination thereof is administered to a human or animal subject.

### 4. Methods of Preparing Compounds

In a further aspect, the present invention provides methods of preparing the compounds, salts, diastereomers, enantiomers, racemates, hydrates, solvates, and prodrugs. In some embodiments, the compounds, salts, diastereomers, enantiomers, racemates, hydrates, solvates, or prodrugs may be prepared by methods including, but not limited to, one or more of the following methods:
(a) optionally converting a compound of the invention into a pharmaceutically acceptable salt;
(b) optionally converting a salt form of a compound of the invention to a non-salt form;
(c) optionally converting an unoxidized form of a compound of the invention into a pharmaceutically acceptable N-oxide;
(d) optionally resolving an individual isomer of a compound of the invention from a mixture of stereoisomers;
(e) optionally converting a non-derivatized compound of the invention into a pharmaceutically acceptable prodrug derivative; and
(f) optionally converting a prodrug derivative of a compound of the invention to its non-derivatized form.

Exemplary methods for preparing the compounds of the invention are described herein, including in the Examples, which will be described in detail below. Some embodiments of the invention provide processes for preparing the compounds of the present invention, as illustrated in Method 1 or Method 2 below.

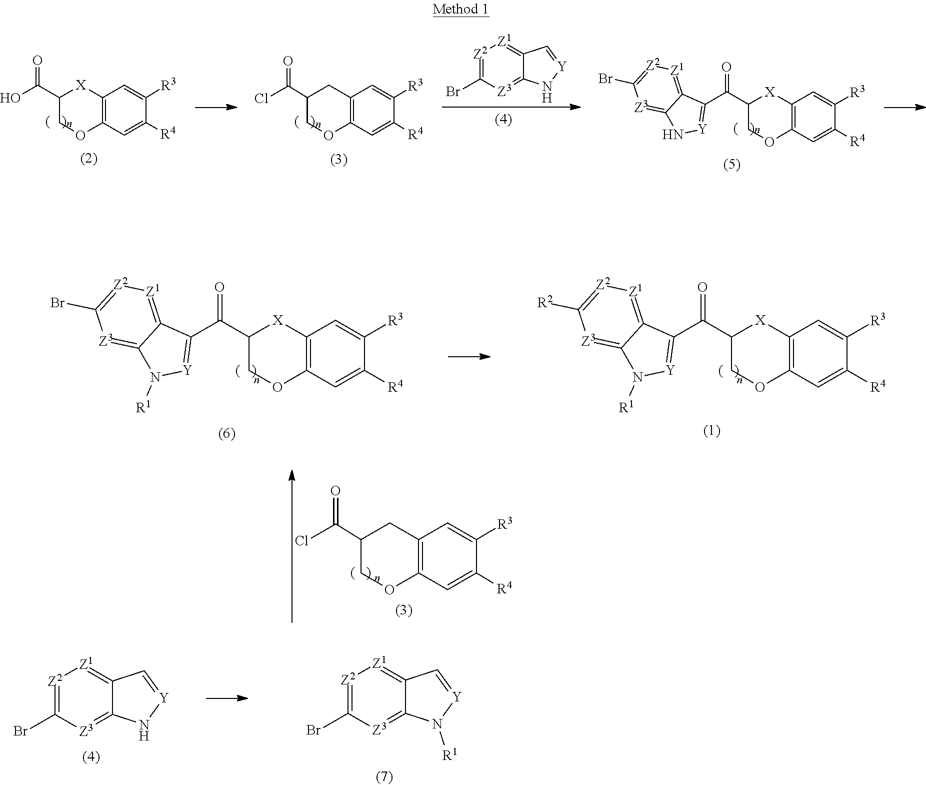

A bicyclic acid (2) was reacted with thionyl chloride or oxalyl chloride in dichloromethane in the presence of catalytic amount of GMF to provide its acyl chloride (3). The resulting acyl chloride (3) was coupled with a bicyclic heteroarly compound (4) in a Friedel-Craft acylation reaction to provide ketone (5) in an organic solvent (dichloromethane or dichloroethane) in the presence of a Lewis acid (aluminum chloride, titanium chloride or stannous chloride). The resulted ketone (5) was reacted with an alkyl bromide, alkyl chloride or alkyl epoxide to afford a N-alkylated compound (6) in an organic solvent (DMF, acetone, dioxane, acetonitrile, dichloromethane or dichloroethane) with an inorganic base (NaHCO₃, Na₂CO₃, K₂CO₃, Cs₂CO₃, K₂HPO₄ or K₃PO₄) at room temperature (RT)-150° C. for 5-24 hours.

The N-alkylated compound (6) also was alternatively prepared starting from a bicyclic heteroarly compound (4) by N-alkylation at the above mentioned reaction condition to give (7), followed by Friedel-Craft acylation reaction with an acyl chloride (3).

The N-alkylated compound (6) was reacted with a boronic acid or a boronic ester with Suzuki reaction condition using a palladium catalyst (Pd(OAc)₂, Pd₂(dba)₃, Pd(PPh₃)₄ or Pd(dppf)Cl₂-DCM) to provide the compound (1) in the presence of an inorganic base (NaHCO₃, Na₂CO₃, K₂CO₃, Cs₂CO₃, CsF, K₂HPO₄ or K₃PO₄) at room temperature (RT)-150° C. for 5-24 hours.

The N-alkylated compound (6) also was alternatively prepared starting from a bicyclic heteroarly compound (4) (Method 2).

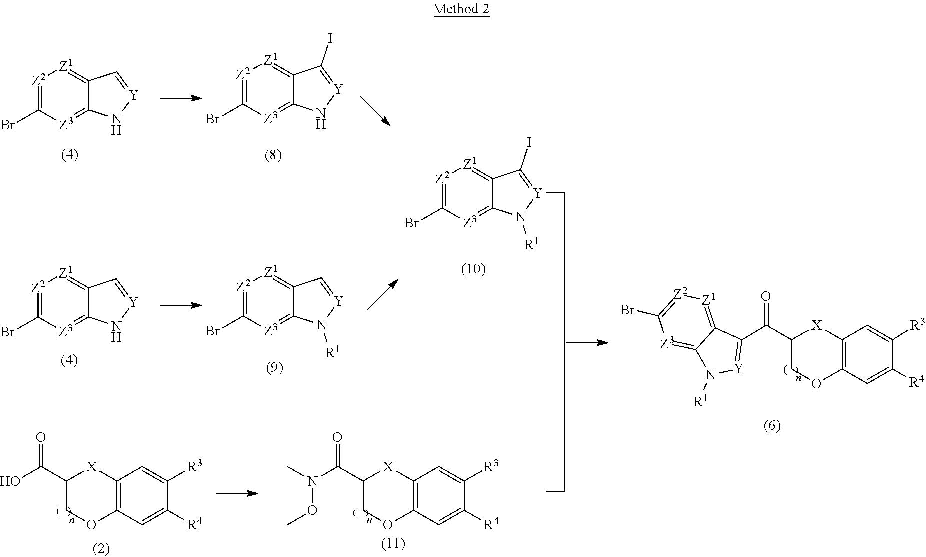

A bicyclic heteroarly compound (4) was reacted with N-iodosuccinimide (NIS) in an organic solvent (DMF, acetone, dioxane, acetonitrile, dichloromethane or dichloroethane) at RT for 2-24 h to provide an iodobicyclic heteroarly compound (8). The iodobicyclic heteroarly compound (8) was reacted with an alkyl bromide, alkyl chloride or alkyl epoxide to afford a N-alkylated compound (10) in an organic solvent (DMF, acetone, dioxane, acetonitrile, dichloromethane or dichloroethane) with an inorganic base (NaHCO₃, Na₂CO₃, K₂CO₃, Cs₂CO₃, K₂HPO₄ or K₃PO₄) at room temperature (RT)-150° C. for 5-24 hours. A N-alkylated compound (10) was alternatively prepared from bicyclic heteroarly compound (4) by N-alkylation to (9), followed by iodonation of (9) using N-iodosuccinimide (NIS) as aforementioned. A bicyclic acid (2) was coupled with N-methoxymethanamine hydrochloride by a coupling reagent (DCC, EDCI, HATU, HBTU, PyBop or PyBrop) in an organic solvent (DMF, tetrahydrofuran, dichloromethane or dichloroethane) at RT for 2-24 h to provide a Weinreb amide (11). The Weinreb amide (11) was converted to a ketone (6) by reacting with iodobicyclic heteroarly compound (10) in the presence of Grignard reagent at low temperature (−78-0° C.) in THF.

Non-limiting examples of the compounds described herein may include:
(6-Chlorochroman-3-yl)-[1-(2-hydroxyethyl)-6-(3-methoxy-1H-pyrazol-4-yl)pyrrolo[3,2-c]pyridin-3-yl]methanone;
(6-Chloro-2H-chromen-3-yl)-[6-(3-chloro-1H-pyrazol-4-yl)-1-(2-hydroxyethyl)pyrrolo[3,2-c]pyridin-3-yl]methanone;
(6-(3-Chloro-1H-pyrazol-4-yl)-1-(2-hydroxyethyl)-1H-pyrrolo[2,3-b]pyridin-3-yl)(6-fluorochroman-3-yl)methanone;
(6-Chlorochroman-3-yl)-[6-(3-chloro-1H-pyrazol-4-yl)-1-(2-hydroxyethyl)pyrrolo[2,3-b]pyridin-3-yl]methanone;
(6-Chlorochroman-3-yl)-[6-(3-chloro-1H-pyrazol-4-yl)-1-(2-hydroxyethyl)pyrrolo[3,2-b]pyridin-3-yl]methanone;
(6-Chlorochroman-3-yl)-[6-(3-chloro-1H-pyrazol-4-yl)-1-(2-hydroxypropyl)pyrrolo[3,2-c]pyridin-3-yl]methanone;
(6-Chlorochroman-3-yl)-[6-(5-chloro-1H-pyrazol-4-yl)-1-[(2S)-2-hydroxypropyl]pyrrolo[3,2-c]pyridin-3-yl]methanone;
(6-Chlorochroman-3-yl)-[6-(3-chloro-1H-pyrazol-4-yl)-1-[(2R)-2-hydroxypropyl]pyrrolo[3,2-c]pyridin-3-yl]methanone;
(6-(5-Chloro-1H-pyrazol-4-yl)-1-((S)-2-hydroxypropyl)-1H-pyrrolo[3,2-c]pyridin-3-yl)((S)-6-chlorochroman-3-yl)methanone;
(6-(5-Chloro-1H-pyrazol-4-yl)-1-((S)-2-hydroxypropyl)-1H-pyrrolo[3,2-c]pyridin-3-yl)((R)-6-chlorochroman-3-yl)methanone;
(6-(5-Chloro-1H-pyrazol-4-yl)-1-((S)-2-hydroxypropyl)-1H-pyrrolo[3,2-c]pyridin-3-yl)((S)-6-chlorochroman-3-yl)methanone methanesulfonic acid;
[6-(3-Chloro-1H-pyrazol-4-yl)-1-(2-hydroxyethyl)pyrrolo[3,2-c]pyridin-3-yl]-(6-fluorochroman-3-yl)methanone;
[6-(3-chloro-1H-pyrazol-4-yl)-1-(2-hydroxypropyl)pyrrolo[3,2-c]pyridin-3-yl]-(6-fluorochroman-3-yl)methanone;
(6-Fluorochroman-3-yl)-[1-(2-hydroxyethyl)-6-(3-methyl-1H-pyrazol-4-yl)pyrrolo[2,3-b]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone hydrochloride;
[6-(3-Chloro-1H-pyrazol-4-yl)-1-[(2S)-2-hydroxypropyl]pyrrolo[3,2-c]pyridin-3-yl]-(6-fluorochroman-3-yl)methanone;
[[6-(3-Chloro-1H-pyrazol-4-yl)-1-[(2R)-2-hydroxypropyl]pyrrolo[3,2-c]pyridin-3-yl]-(6-fluorochroman-3-yl)methanone;
[6-(3-Chloro-1H-pyrazol-4-yl)-1-(2-hydroxyethyl)pyrrolo[2,3-b]pyridin-3-yl]-(6-methoxychroman-3-yl)methanone;
[6-(3-Chloro-1H-pyrazol-4-yl)-1-(2-hydroxyethyl)pyrrolo[3,2-c]pyridin-3-yl]-(6-methoxychroman-3-yl)methanone;
[6-(5-Fluoro-1H-pyrazol-4-yl)-1-(2-hydroxyethyl)pyrrolo[3,2-c]pyridin-3-yl]-(6-methoxychroman-3-yl)methanone;
[6-(5-Fluoro-1H-pyrazol-4-yl)-1-[(2S)-2-hydroxypropyl]pyrrolo[3,2-c]pyridin-3-yl]-(6-methoxychroman-3-yl)methanone;
[6-(5-Fluoro-1H-pyrazol-4-yl)-1-[(2S)-2-hydroxypropyl]pyrrolo[3,2-c]pyridin-3-yl]-(6-methoxychroman-3-yl)methanone;
[6-(5-Chloro-1H-pyrazol-4-yl)-1-[(2S)-2-hydroxypropyl]pyrrolo[3,2-c]pyridin-3-yl]-(6-methoxychroman-3-yl)methanone;
[6-(5-Fluoro-1H-pyrazol-4-yl)-1-(2-hydroxy-2-methyl-propyl)pyrrolo[3,2-c]pyridin-3-yl]-(6-methoxychroman-3-yl)methanone;
[6-(5-Chloro-1H-pyrazol-4-yl)-1-(2-hydroxy-2-methyl-propyl)pyrrolo[3,2-c]pyridin-3-yl]-(6-methoxychroman-3-yl)methanone;
[(3S)-6-Chlorochroman-3-yl]-[6-(5-fluoro-1H-pyrazol-4-yl)-1-[(2S)-2-hydroxypropyl]pyrrolo[3,2-c]pyridin-3-yl]methanone;
[(3S)-6-Chlorochroman-3-yl]-[6-(5-fluoro-1H-pyrazol-4-yl)-1-(2-hydroxy-2-methyl-propyl)pyrrolo[3,2-c]pyridin-3-yl]methanone;
[(3S)-6-Chlorochroman-3-yl]-[6-(5-chloro-1H-pyrazol-4-yl)-1-(2-hydroxy-2-methyl-propyl)pyrrolo[3,2-c]pyridin-3-yl]methanone;
[(3S)-6-Chlorochroman-3-yl]-[1-[(2S)-2-hydroxypropyl]-6-(5-methoxy-1H-pyrazol-4-yl)pyrrolo[3,2-c]pyridin-3-yl]methanone;
[(3R)-6-Chlorochroman-3-yl]-[1-[(2R)-2-hydroxypropyl]-6-(5-methoxy-1H-pyrazol-4-yl)pyrrolo[3,2-c]pyridin-3-yl]methanone;
(6-Chlorochroman-3-yl)-[6-(5-chloro-1H-pyrazol-4-yl)-1-[(2S)-2-hydroxybutyl]pyrrolo[3,2-c]pyridin-3-yl]methanone;
(6-Chlorochroman-3-yl)-[6-(5-fluoro-1H-pyrazol-4-yl)-1-[(2S)-2-hydroxybutyl]pyrrolo[3,2-c]pyridin-3-yl]methanone;
(6-Chlorochroman-3-yl)-[6-(5-fluoro-1H-pyrazol-4-yl)-1-[(3-hydroxycyclobutyl)methyl]pyrrolo[3,2-c]pyridin-3-yl]methanone;
(6-Chlorochroman-3-yl)-[6-(5-chloro-1H-pyrazol-4-yl)-1-[(3-hydroxycyclobutyl)methyl]pyrrolo[3,2-c]pyridin-3-yl]methanone;
(6-Chlorochroman-3-yl)-[6-(3-chloro-1H-pyrazol-4-yl)-1-(2-hydroxypropyl)indol-3-yl]methanone;
(6-(3-Chloro-1H-pyrazol-4-yl)-1-(2-hydroxypropyl)-1H-indol-3-yl)((S)-6-chlorochroman-3-yl)methanone;
(6-(3-Chloro-1H-pyrazol-4-yl)-1-(2-hydroxypropyl)-1H-indol-3-yl)((S)-6-chlorochroman-3-yl)methanone;
((S)-6-Chlorochroman-3-yl)(1-(2-hydroxypropyl)-6-(3-methoxy-1H-pyrazol-4-yl)-1H-indol-3-yl)methanone;
(6-Chlorochroman-3-yl)-[1-[(2S)-2-hydroxypropyl]-6-(3-methoxy-1H-pyrazol-4-yl)indol-3-yl]methanone;
(6-Chlorochroman-3-yl)-[1-[(2R)-2-hydroxypropyl]-6-(3-methoxy-1H-pyrazol-4-yl)indol-3-yl]methanone;
(6-Chlorochroman-3-yl)-[2-(5-chloro-1H-pyrazol-4-yl)-7-[(2S)-2-hydroxypropyl]pyrrolo[2,3-d]pyrimidin-5-yl]methanone;
(2-(5-Chloro-1H-pyrazol-4-yl)-7-((S)-2-hydroxypropyl)-7H-pyrrolo[2,3-d]pyrimidin-5-yl)((S)-6-chlorochroman-3-yl)methanone;
(6-Chlorochroman-3-yl)-[2-(5-fluoro-1H-pyrazol-4-yl)-7-[(2S)-2-hydroxypropyl]pyrrolo[2,3-d]pyrimidin-5-yl]methanone;
(2-(5-Fluoro-1H-pyrazol-4-yl)-7-((R)-2-hydroxypropyl)-7H-pyrrolo[2,3-d]pyrimidin-5-yl)(6-fluorochroman-3-yl)methanone;
(2-(5-Fluoro-1H-pyrazol-4-yl)-7-((R)-2-hydroxypropyl)-7H-pyrrolo[2,3-d]pyrimidin-5-yl)((S)-6-fluorochroman-3-yl)methanone;
(2-(5-Fluoro-1H-pyrazol-4-yl)-7-((R)-2-hydroxypropyl)-7H-pyrrolo[2,3-d]pyrimidin-5-yl)((S)-6-fluorochroman-3-yl)methanone hydrochloride;
(2-(5-Fluoro-1H-pyrazol-4-yl)-7-((R)-2-hydroxypropyl)-7H-pyrrolo[2,3-d]pyrimidin-5-yl)((S)-6-fluorochroman-3-yl)methanone methanesulfonic acid;
(6-Fluorochroman-3-yl)(7-(2-hydroxy-2-methylpropyl)-2-(5-methoxy-1H-pyrazol-4-yl)-7H-pyrrolo[2,3-d]pyrimidin-5-yl)methanone;
(6-Chlorochroman-3-yl)-[7-[(2S)-2-hydroxypropyl]-2-(5-methoxy-1H-pyrazol-4-yl)pyrrolo[2,3-d]pyrimidin-5-yl]methanone;
(6-Chlorochroman-3-yl)-[2-(5-fluoro-1H-pyrazol-4-yl)-7-(2-hydroxy-2-methyl-propyl)pyrrolo[2,3-d]pyrimidin-5-yl]methanone;
(S)-(6-Chlorochroman-3-yl)(2-(5-fluoro-1H-pyrazol-4-yl)-7-(2-hydroxy-2-methylpropyl)-7H-pyrrolo[2,3-d]pyrimidin-5-yl)methanone;
(S)-(6-Chlorochroman-3-yl)(2-(5-fluoro-1H-pyrazol-4-yl)-7-(2-hydroxy-2-methylpropyl)-7H-pyrrolo[2,3-d]pyrimidin-5-yl)methanone methanesulfonic acid;
(6-Chlorochroman-3-yl)-[2-(5-chloro-1H-pyrazol-4-yl)-7-(2-hydroxy-2-methyl-propyl)pyrrolo[2,3-d]pyrimidin-5-yl]methanone;
(6-Chlorochroman-3-yl)-[7-(2-hydroxy-2-methyl-propyl)-2-(5-methoxy-1H-pyrazol-4-yl)pyrrolo[2,3-d]pyrimidin-5-yl]methanone;
(6-Chlorochroman-3-yl)(1-(2-hydroxyethyl)-6-(5-methoxy-1H-pyrazol-4-yl)-1H-pyrrolo[3,2-c]pyridin-3-yl)methanone hydrochloride;
(6-Chlorochroman-3-yl)(1-(2-hydroxyethyl)-6-(5-methoxy-1H-pyrazol-4-yl)-1H-pyrrolo[3,2-c]pyridin-3-yl)methanone methanesulfonic acid;
(2,3-Dihydrobenzo[b][1,4]dioxin-2-yl)(1-(2-hydroxyethyl)-6-(1H-pyrazol-4-yl)-1H-indol-3-yl)methanone;
Chroman-3-yl-[1-(2-hydroxyethyl)-6-(1H-pyrazol-4-yl)indol-3-yl]methanone;
2,3-Dihydro-1,4-benzodioxin-3-yl-[1-(2-hydroxyethyl)-6-(1H-pyrrolo[2,3-b]pyridin-3-yl)indol-3-yl]methanone;
[6-(3-Chloro-1H-pyrazol-4-yl)-1-(2-hydroxyethyl)indol-3-yl]-(2,3-dihydro-1,4-benzodioxin-3-yl)methanone;
Chroman-3-yl-[1-(2-hydroxyethyl)-6-(1H-pyrrolo[2,3-b]pyridin-3-yl)indol-3-yl]methanone;
[6-(3-Chloro-1H-pyrazol-4-yl)-1-(2-hydroxyethyl)indol-3-yl]-chroman-3-yl-methanone;
(6-Fluorochroman-3-yl)-[1-(2-hydroxyethyl)-6-(1H-pyrazol-4-yl)indol-3-yl]methanone;
[1-(2-Hydroxyethyl)-6-(1H-pyrazol-4-yl)indol-3-yl]-(7-methoxychroman-3-yl)methanone;
(6-Fluoro-2,3-dihydro-1,4-benzodioxin-3-yl)-[1-(2-hydroxyethyl)-6-(1H-pyrazol-4-yl)indol-3-yl]methanone;
(6-Chloro-2,3-dihydro-1,4-benzodioxin-2-yl)-[1-(2-hydroxyethyl)-6-(1H-pyrazol-4-yl)indol-3-yl]methanone;
(6-Fluoro-2,3-dihydro-1,4-benzodioxin-2-yl)-[1-(2-hydroxyethyl)-6-(1H-pyrazol-4-yl)indol-3-yl]methanone;
(6,7-Dichloro-2,3-dihydro-1,4-benzodioxin-3-yl)-[1-(2-hydroxyethyl)-6-(1H-pyrazol-4-yl)indol-3-yl]methanone;
(6-Chloro-2,3-dihydro-1,4-benzodioxin-3-yl)-[1-(2-hydroxyethyl)-6-(1H-pyrazol-4-yl)indol-3-yl]methanone;
[11-(2-Hydroxyethyl)-6-(1H-pyrazol-4-yl)indol-3-yl]-(6-methoxy-2,3-dihydro-1,4-benzodioxin-3-yl)methanone;
[6-(3-Chloro-1H-pyrazol-4-yl)-1-(2-hydroxyethyl)indol-3-yl]-(6-methoxychroman-3-yl)methanone;
(6-Chlorochroman-3-yl)(6-(3-fluoro-1H-pyrazol-4-yl)-1-(2-hydroxyethyl)-1H-indol-3-yl)methanone;
(6-Chlorochroman-3-yl)(1-(2-hydroxyethyl)-6-(3-methoxy-1H-pyrazol-4-yl)-1H-indol-3-yl)methanone;
(S)-(6-Chlorochroman-3-yl)(1-(2-hydroxyethyl)-6-(3-methoxy-1H-pyrazol-4-yl)-1H-indol-3-yl)methanone;
(R)-(6-Chlorochroman-3-yl)(1-(2-hydroxyethyl)-6-(3-methoxy-1H-pyrazol-4-yl)-1H-indol-3-yl)methanone;
(6-Fluorochroman-3-yl)(1-(2-hydroxyethyl)-6-(3-methoxy-1H-pyrazol-4-yl)-1H-indol-3-yl)methanone;
(S)-(6-Fluorochroman-3-yl)(1-(2-hydroxyethyl)-6-(3-methoxy-1H-pyrazol-4-yl)-1H-indol-3-yl)methanone;
(R)-(6-Fluorochroman-3-yl)(1-(2-hydroxyethyl)-6-(3-methoxy-1H-pyrazol-4-yl)-1H-indol-3-yl)methanone;
Chroman-3-yl(1-(2-hydroxyethyl)-6-(1H-pyrazol-4-yl)-1H-indol-3-yl)methanone;
Chroman-3-yl(1-(2-methoxyethyl)-6-(1H-pyrazol-4-yl)-1H-indazol-3-yl)methanone;
(S)-(2,3-Dihydrobenzo[b][1,4]dioxin-2-yl)(1-(2-methoxyethyl)-6-(1H-pyrazol-4-yl)-1H-indazol-3-yl)methanone;
(6-Fluorochroman-3-yl)(1-(2-hydroxyethyl)-6-(3-(trifluoromethyl)-1H-pyrazol-4-yl)-1H-indazol-3-yl)methanone;
(6-Fluorochroman-3-yl)(1-(2-hydroxyethyl)-6-(3-methoxy-1H-pyrazol-4-yl)-1H-indazol-3-yl)methanone;
(6-Fluorochroman-3-yl)(1-(2-hydroxyethyl)-6-(3-isopropyl-1H-pyrazol-4-yl)-1H-indazol-3-yl)methanone;
2-(3-(6-Chlorochromane-3-carbonyl)-6-(3-methoxy-1H-pyrazol-4-yl)-1H-indazol-1-yl)acetonitrile;
(2,3-Dihydrobenzofuran-2-yl)(1-(2-hydroxyethyl)-6-(3-methoxy-1H-pyrazol-4-yl)-1H-indazol-3-yl)methanone;
(1-(2-Hydroxyethyl)-6-(3-methoxy-1H-pyrazol-4-yl)-1H-indazol-3-yl)(2-methyl-2,3-dihydrobenzo[b][1,4]dioxin-2-yl)methanone;
Benzofuran-3-yl(1-(2-hydroxyethyl)-6-(3-methoxy-1H-pyrazol-4-yl)-1H-indazol-3-yl)methanone;
(6-Chlorochroman-3-yl)(1-(2-hydroxypropyl)-6-(3-methoxy-1H-pyrazol-4-yl)-1H-indazol-3-yl)methanone;
(6-Chlorochroman-3-yl)(1-((R)-2-hydroxypropyl)-6-(3-methoxy-1H-pyrazol-4-yl)-1H-indazol-3-yl)methanone;
(6-Chlorochroman-3-yl)(1-((S)-2-hydroxypropyl)-6-(3-methoxy-1H-pyrazol-4-yl)-1H-indazol-3-yl)methanone;
(6-Fluorochroman-3-yl)(1-(2-hydroxyethyl)-6-(1H-pyrazol-4-yl)-1H-indazol-3-yl)methanone;
(Chroman-3-yl(1-(2-hydroxyethyl)-6-(1H-pyrazol-4-yl)-1H-indazol-3-yl)methanone;
(7-Fluoro-2,3-dihydrobenzo[b][1,4]dioxin-2-yl)(1-(2-hydroxyethyl)-6-(1H-pyrazol-4-yl)-1H-indazol-3-yl)methanone; and
(7-Chloro-2,3-dihydrobenzo[b][1,4]dioxin-2-yl)(1-(2-hydroxyethyl)-6-(1H-pyrazol-4-yl)-1H-indazol-3-yl)methanone.

### EXAMPLES

The present invention is further exemplified by the following examples that illustrate the preparation of compounds of FIG. 1 according to the invention. The examples are for illustrative purpose only and are not intended, nor should they be construed as limiting the invention in any manner. Those skilled in the art will appreciate that variations and modifications can be made without changing the scope of the invention.

Nuclear magnetic resonance (NMR) and mass spectrometry (MS) spectra obtained for compounds described in the examples below and those described herein were consistent with that of the compounds of formulae herein.

Liquid chromatography-mass spectrometry (LC-MS) Method:
1. Samples are run on Agilent Technologies 6120 MSD system with a Zorbax Eclipse XDB-C18 (3.5μ) reverse phase column (4.6×50 mm) run at room temperature with flow rate of 1.5 mL/minute.
2. The mobile phase uses solvent A (water/0.1% formic acid) and solvent B (acetonitrile/0.1% formic acid): 95%/5% to 0%/100% (A/B) for 5 minute.
3. The mass spectra (m/z) were recorded using electrospray ionization (ESI).
4. Ionization data was rounded to the nearest integer.

### Proton NMR Spectra:

Unless otherwise indicated, all ¹H NMR spectra are run on a Varian series Mercury 400 or 500 MHz. All observed protons are reported as parts-per-million (ppm) downfield from tetramethylsilane using conventional abbreviations for designation of major peaks: e.g., s (singlet), d (doublet), t (triplet), q (quartet), m (multiplet) and brs (broad singlet).

### Example 1: Preparation of Compounds

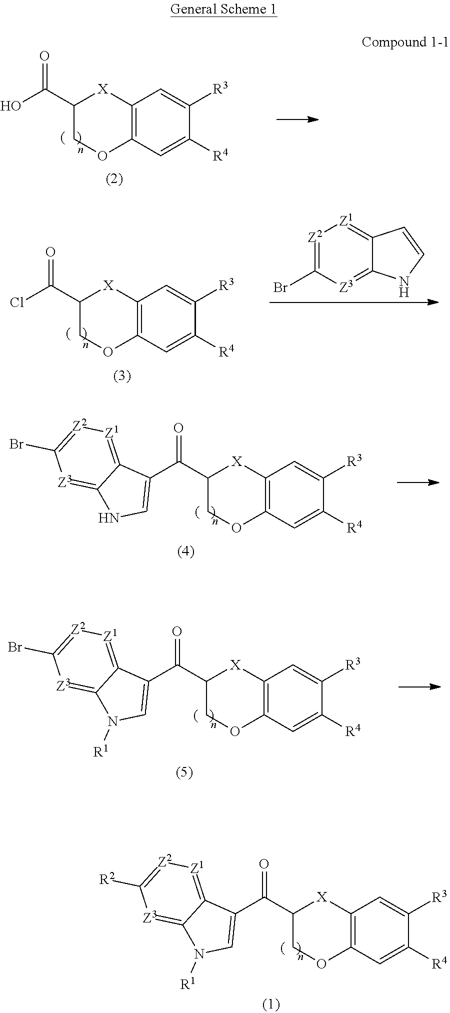

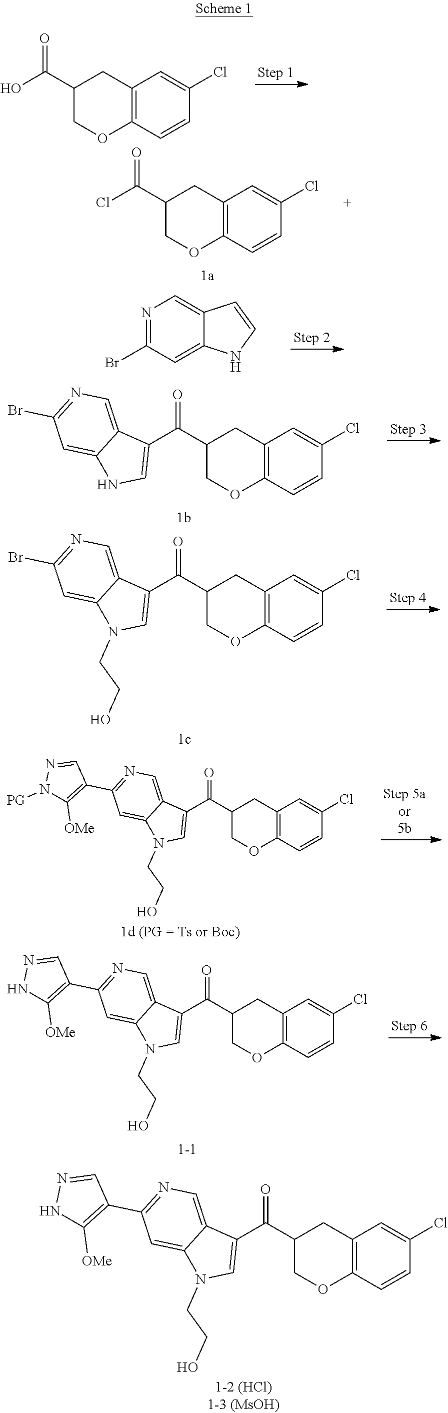

Step 1: A mixture of 6-chlorochromane-3-carboxylic acid (6.0 g, 28.2 mmol), thionyl chloride (20.98 mL, 282.18 mmol), and DMF (0.1 mL, catalyst) in toluene (60 mL) was heated to reflux with stirring for 3 hours. The mixture was cooled to RT and concentrated to remove volatile organic solvents. The crude was dissolved in acetonitrile (100 mL) and concentrated to provide 1a as a pale blown oil (crude), which was used in the next step without further purification.

Step 2: A mixture of 1a (5.4 g, 23 mmol) and 6-bromo-1H-pyrrolo[3,2-c]pyridine (3.7 g, 19 mmol) in dichloromethane (60 mL) was cooled in an ice bath, and aluminum chloride (9.4 g, 70 mmol) was added. The mixture was stirred for 1 hour at 0° C. The mixture was quenched with a saturated NH₄Cl solution (100 mL) at 0° C. to form solid precipitation. The mixture was then warmed to room temperature and stirred for additional 2 hours. The solids were collected by filtration and then rinsed with methanol. The resulting solids were dried under high vacuum to provide 1b as a white solid in 80% yield (7.4 g). LC/MS found 391.0 and 393.0 [M+H]⁺.

Step 3: A mixture of 1b (800 mg, 2.1 mmol), potassium carbonate (1.76 g, 1.3 mmol, 770 μL), and 2-chloro-N,N-dimethylethanamine hydrochloride (690 mg, 6.4 mmol) in DMF (20 mL) was heated to 70° C. and stirred overnight. The mixture was poured over water (100 mL) and extracted with EtOAc (100 mL). The organic layer was separated, washed with brine (100 mL), dried over Na₂SO₄, filtered, and concentrated. The residue was purified by flash column chromatography on silica gel column using a gradient of 20-100% EtOAc in hexane to give 1c as a white solid in 20% yield (202 mg). LC/MS found 463.1 and 465.0 [M+H]⁺.

Step 4: A mixture of Intermediate 1c (202 mg, 430 μmol), [3-methoxy-1-(p-tolylsulfonyl)pyrazol-4-yl]boronic acid (190 mg, 650 μmol), potassium carbonate (2 M in water, 540 μL), and tetrakis(triphenylphosphine)palladium (25.0 mg, 22 μmol) in 1,4-dioxane (10 mL) was degassed via sparging with argon for 5 minutes, and the reaction mixture was heated to 70° C. and shaken overnight. The mixture was cooled to room temperature, concentrated, poured over water (50 mL), and extracted twice with EtOAc (50 mL). The combined organic layers were dried over Na₂SO₄, filtered, and concentrated to afford 1d, which was used for the next step without further purification.

Step 5a for a tosyl (Ts) protected pyrazole compound: A solution of 1d (crude, 230 mg) in THF (1 mL) was cooled on an ice bath and treated with 2N sodium hydroxide solution (2.0 mL). The solution was stirred at 0° C. 30 minutes, and it was neutralized with 1N HCl solution (4.0 mL), diluted with EtOAc (30 mL), and saturated Na₂CO₃ solution (30 mL). The aqueous phase was separated and extracted with EtOAc. The organic layers were combined, dried over Na₂SO₄, filtered, treated with silica gel, and evaporated under reduced pressure. The material was purified by silica gel column chromatography to provide a compound 1-1 (6-chlorochroman-3-yl)-[1-[2-(dimethylamino)ethyl]-6-(3-methoxy-1H-pyrazol-4-yl)pyrrolo[3,2-c]pyridin-3-yl]methanone (101 mg, 210 μmol, 58% yield for step 4 and 5a) as a white solid. LC/MS found 480.2 [M+H]⁺.

Step 6a: ((6-chlorochroman-3-yl)(1-(2-(dimethylamino)ethyl)-6-(5-methoxy-1H-pyrazol-4-yl)-1H-pyrrolo[3,2-c]pyridin-3-yl)methanone hydrochloride): To a solution of 1-1 (50 mg, 104 μmol) in acetone (1 mL), was slowly added a 4M solution of HCl in dioxane (55 μL) at room temperature to form solid precipitation. After being stirred for additional 1 hour at room temperature, was added ter-butylethylether (2 mL) for a complete precipitation. The solids were collected by filtration under nitrogen atmosphere, rinsed with ter-butylethylether, and dried under vacuum to give 1-59 (47.5 mg, 82%) as white powder. LC/MS found 480.2 [M+H]⁺.

Step 6a: ((6-chlorochroman-3-yl)(1-(2-(dimethylamino)ethyl)-6-(5-methoxy-1H-pyrazol-4-yl)-1H-pyrrolo[3,2-c]pyridin-3-yl)methanone metanesulfonic acid): To a solution of 1-1 (50 mg, 104 μmol) in acetone (1 mL), was slowly added a 1M solution of metanesulfonic acid in acetone (220 μL) at room temperature to form solid precipitation. After being stirred for additional 1 hour at room temperature, was added ter-butylethylether (2 mL) for a complete precipitation. The solids were collected by filtration under nitrogen atmosphere, rinsed with ter-butylethylether, and dried under vacuum to give 1-60 (60.3 mg, 90%) as white powder. LC/MS found 480.2 [M+H]⁺.

Step 5b for a Boc protected pyrazole compound: A solution of crude tert-butyl 3-chloro-4-(3-(6-chloro-2H-chromene-3-carbonyl)-1-(2-hydroxyethyl)-1H-pyrrolo[3,2-c]pyridin-6-yl)-1H-pyrazole-1-carboxylate (40.7 mg) in DCM (1.0 mL) was treated with TFA (0.5 mL) and stirred at ambient temperature. After being stirred for 2 h at rt, the reaction mixture was concentrated under reduced pressure to provide a residue. The residue was dissolved in EtOAc (10 mL) and washed with 2M Na₂CO₃ solution (10 mL). The aqueous layer was separated and extracted with EtOAc (5 mL), and the combined organics were dried over Na₂SO₄, filtered, treated with silica gel, and evaporated under reduced pressure. The material was purified by silica gel column chromatography to provide a compound 1-1 (6-chloro-2H-chromen-3-yl)-[6-(3-chloro-1H-pyrazol-4-yl)-1-(2-hydroxyethyl)pyrrolo[3,2-c]pyridin-3-yl]methanone (33.0 mg, 41.8 μmol; 55% yield for steps 4 and 5b) as a pale brown solid. LC/MS found 458.1 [M+H]⁺.

The following compounds 1-2 to 1-60 shown in Table 1 were prepared by the method (General Scheme 1) similar to that described for the preparation of compound 1-1 using appropriate (4) with an appropriate boronic acid/ester (R²) and appropriate R¹ substituent.

TABLE 1

| | | | | | MS |
|---|---|---|---|---|---|
| | R¹ | R² | | | found |
| Compd | block | boronic acid | (4) | Structure | [M + H]⁺ |
| 1-1 | 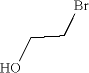 | 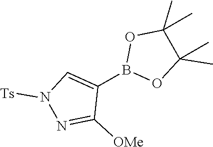 | 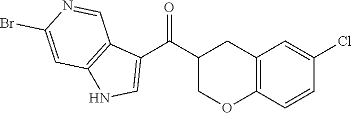 [SMILES: O=C(c1c[nH]c2cc(Br)ncc12)C1COc2ccc(Cl)cc2C1] | 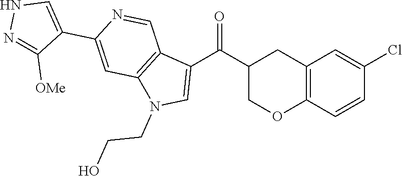 [SMILES: COc1n[nH]cc1-c1cc2c(cn1)c(C(=O)C1COc3ccc(Cl)cc3C1)cn2CCO] | 453.2 |
| 1-2 | 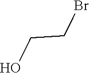 | 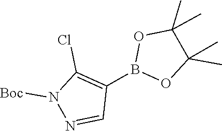 | 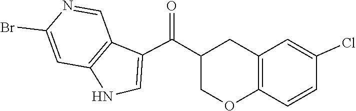 [SMILES: O=C(c1c[nH]c2cc(Br)ncc12)C1COc2ccc(Cl)cc2C1] | 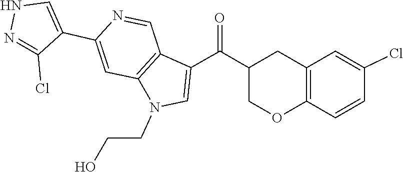 [SMILES: O=C(c1cn(CCO)c2cc(-c3c[nH]nc3Cl)ncc12)C1COc2ccc(Cl)cc2C1] | 458.1 |
| 1-3 | 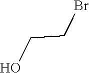 | 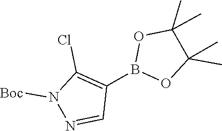 | 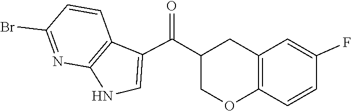 [SMILES: O=C(c1c[nH]c2nc(Br)ccc12)C1COc2ccc(F)cc2C1] | 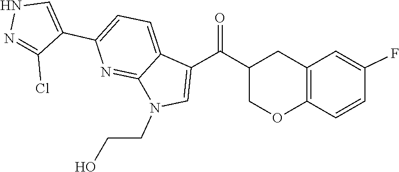 [SMILES: O=C(c1cn(CCO)c2nc(-c3c[nH]nc3Cl)ccc12)C1COc2ccc(F)cc2C1] | 441.1 |
| 1-4 | 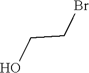 | 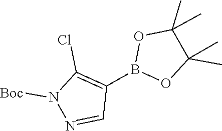 | 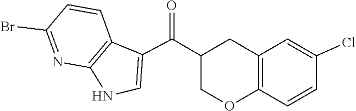 [SMILES: O=C(c1c[nH]c2nc(Br)ccc12)C1COc2ccc(Cl)cc2C1] | 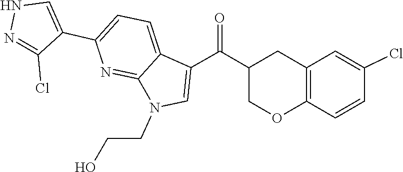 [SMILES: O=C(c1cn(CCO)c2nc(-c3c[nH]nc3Cl)ccc12)C1COc2ccc(Cl)cc2C1] | 457.1 |
| 1-5 | 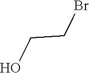 | 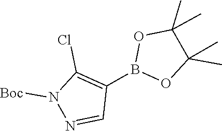 | 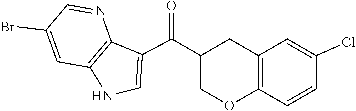 [SMILES: O=C(c1c[nH]c2cc(Br)cnc12)C1COc2ccc(Cl)cc2C1] | 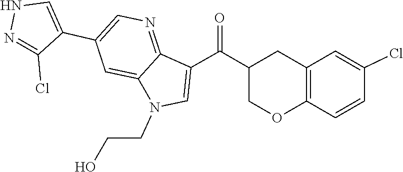 [SMILES: O=C(c1cn(CCO)c2cc(-c3c[nH]nc3Cl)cnc12)C1COc2ccc(Cl)cc2C1] | 457.1 |
| 1-6 | 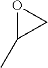 | 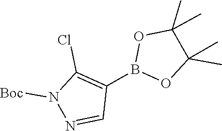 | 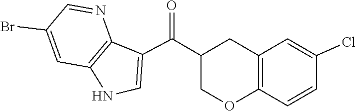 [SMILES: O=C(c1c[nH]c2cc(Br)cnc12)C1COc2ccc(Cl)cc2C1] | 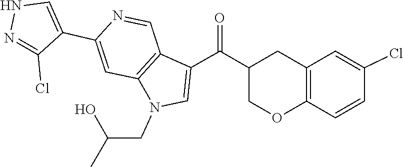 [SMILES: CC(O)Cn1cc(C(=O)C2COc3ccc(Cl)cc3C2)c2cnc(-c3c[nH]nc3Cl)cc21] | 471.2 |
| 1-7 | 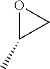 | 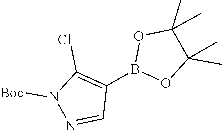 | 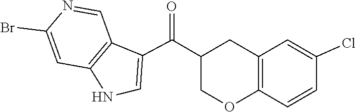 [SMILES: O=C(c1c[nH]c2cc(Br)ncc12)C1COc2ccc(Cl)cc2C1] | 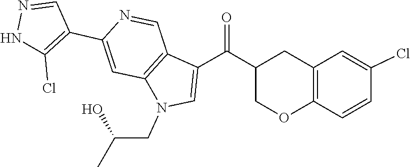 [SMILES: C[C@H](O)Cn1cc(C(=O)C2COc3ccc(Cl)cc3C2)c2cnc(-c3cn[nH]c3Cl)cc21] | 471.1 |
| 1-8 | 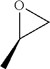 | 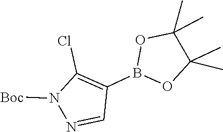 | 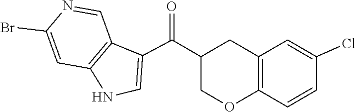 [SMILES: O=C(c1c[nH]c2cc(Br)ncc12)C1COc2ccc(Cl)cc2C1] | 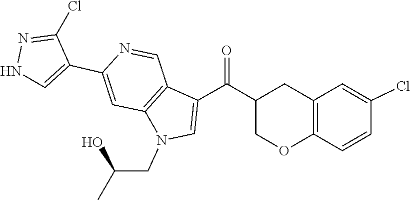 [SMILES: C[C@@H](O)Cn1cc(C(=O)C2COc3ccc(Cl)cc3C2)c2cnc(-c3c[nH]nc3Cl)cc21] | 471.2 |
| 1-9 | 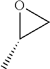 | 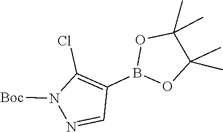 | 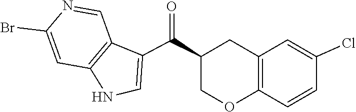 [SMILES: O=C(c1c[nH]c2cc(Br)ncc12)[C@@H]1COc2ccc(Cl)cc2C1] | 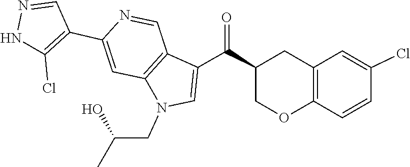 [SMILES: C[C@H](O)Cn1cc(C(=O)[C@@H]2COc3ccc(Cl)cc3C2)c2cnc(-c3cn[nH]c3Cl)cc21] | 471.1 |
| 1-10 | 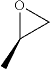 | 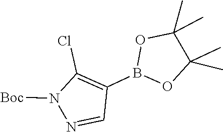 | 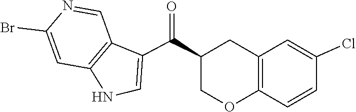 [SMILES: O=C(c1c[nH]c2cc(Br)ncc12)[C@@H]1COc2ccc(Cl)cc2C1] | 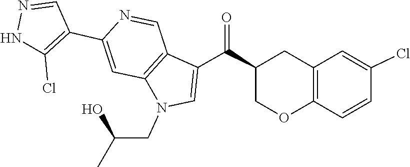 [SMILES: C[C@@H](O)Cn1cc(C(=O)[C@@H]2COc3ccc(Cl)cc3C2)c2cnc(-c3cn[nH]c3Cl)cc21] | 471.3 |
| | | | | ¹H NMR (500 MHz, DMSO-d6) δ ppm | |
| | | | | 13.27 (br s, 1 H) 9.31 (d, J = 0.8 Hz, 1 H) | |
| | | | | 8.67 (s, 1 H) 8.32 (s, 1 H) 8.00 (s, 1 H) | |
| | | | | 7.24 (d, J = 2.4 Hz, 1 H) 7.14 (dd, | |
| | | | | J = 8.7, 2.6 Hz, 1 H) 6.83 (d, J = 8.7 Hz, | |
| | | | | 1 H) 5.04 (d, J = 4.7 Hz, 1 H) 4.45-4.54 | |
| | | | | (m, 1 H) 4.20-4.30 (m, 1 H) 4.03- | |
| | | | | 4.17 (m, 3 H) 3.85 (tdd, J = 9.5, 9.5, 5.7, | |
| | | | | 3.4 Hz, 1 H) 2.94-3.12 (m, 2 H) | |
| | | | | 1.08-1.15 (m, 3 H) | |
| 1-11 | 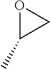 | 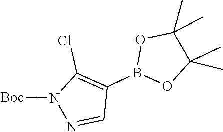 | 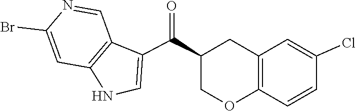 [SMILES: O=C(c1c[nH]c2cc(Br)ncc12)[C@@H]1COc2ccc(Cl)cc2C1] | 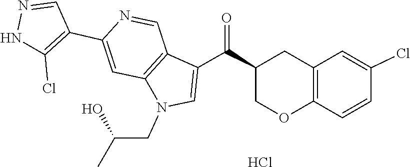 [SMILES: C[C@H](O)Cn1cc(C(=O)[C@@H]2COc3ccc(Cl)cc3C2)c2cnc(-c3cn[nH]c3Cl)cc21 ; Cl] | 471.1 |
| 1-12 | 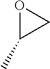 | 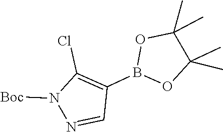 | 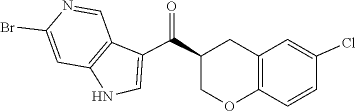 [SMILES: O=C(c1c[nH]c2cc(Br)ncc12)[C@@H]1COc2ccc(Cl)cc2C1] | 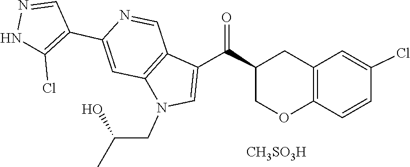 | 471.1 |
| 1-13 | 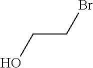 | 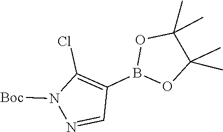 | 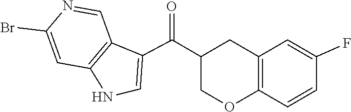 [SMILES: O=C(c1c[nH]c2cc(Br)ncc12)C1COc2ccc(F)cc2C1] | 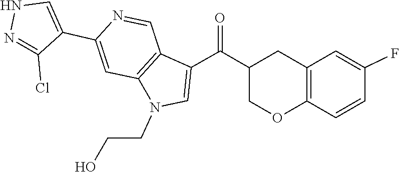 [SMILES: O=C(c1cn(CCO)c2cc(-c3c[nH]nc3Cl)ncc12)C1COc2ccc(F)cc2C1] | 441.1 |
| 1-14 | 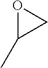 | 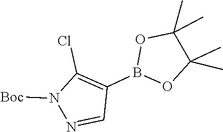 | 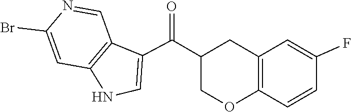 [SMILES: O=C(c1c[nH]c2cc(Br)ncc12)C1COc2ccc(F)cc2C1] | 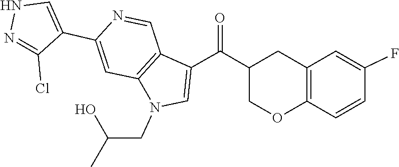 [SMILES: CC(O)Cn1cc(C(=O)C2COc3ccc(F)cc3C2)c2cnc(-c3c[nH]nc3Cl)cc21] | 455.2 |
| 1-15 | 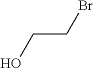 | 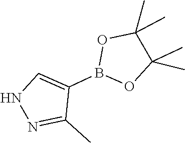 | 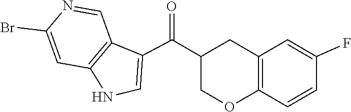 [SMILES: O=C(c1c[nH]c2cc(Br)ncc12)C1COc2ccc(F)cc2C1] | 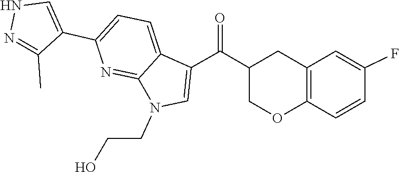 [SMILES: Cc1n[nH]cc1-c1ccc2c(C(=O)C3COc4ccc(F)cc4C3)cn(CCO)c2n1] | 421.2 |
| 1-16 | 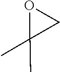 | 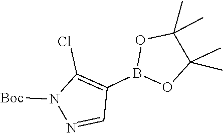 | 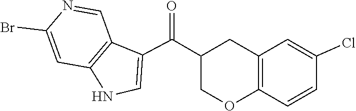 [SMILES: O=C(c1c[nH]c2cc(Br)ncc12)C1COc2ccc(Cl)cc2C1] | 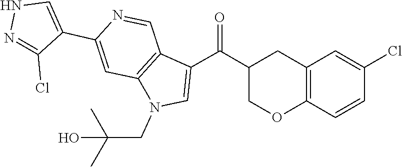 [SMILES: CC(C)(O)Cn1cc(C(=O)C2COc3ccc(Cl)cc3C2)c2cnc(-c3c[nH]nc3Cl)cc21] | 486.2 |
| 1-1 | 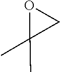 | 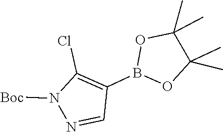 | 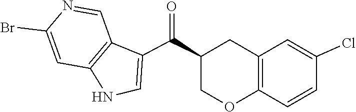 [SMILES: O=C(c1c[nH]c2cc(Br)ncc12)[C@@H]1COc2ccc(Cl)cc2C1] | 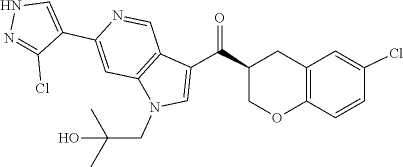 [SMILES: CC(C)(O)Cn1cc(C(=O)[C@@H]2COc3ccc(Cl)cc3C2)c2cnc(-c3c[nH]nc3Cl)cc21] | 486.2 |
| 1-18 | 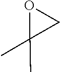 | 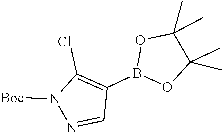 | 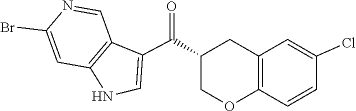 [SMILES: O=C(c1c[nH]c2cc(Br)ncc12)[C@H]1COc2ccc(Cl)cc2C1] | 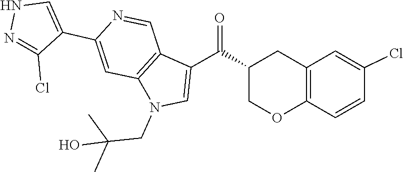 [SMILES: CC(C)(O)Cn1cc(C(=O)[C@H]2COc3ccc(Cl)cc3C2)c2cnc(-c3c[nH]nc3Cl)cc21] | 486.2 |
| 1-19 | 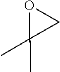 | 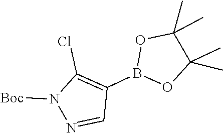 | 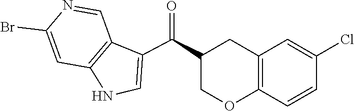 | 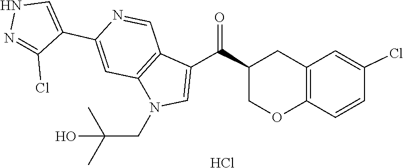 [SMILES: CC(C)(O)Cn1cc(C(=O)[C@@H]2COc3ccc(Cl)cc3C2)c2cnc(-c3c[nH]nc3Cl)cc21 ; Cl] | 486.2 |
| 1-20 | 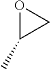 | 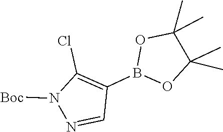 | 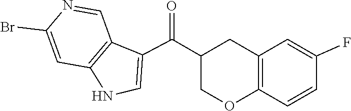 [SMILES: O=C(c1c[nH]c2cc(Br)ncc12)C1COc2ccc(F)cc2C1] | 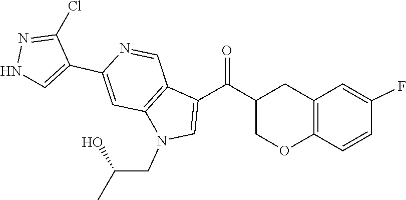 [SMILES: C[C@H](O)Cn1cc(C(=O)C2COc3ccc(F)cc3C2)c2cnc(-c3c[nH]nc3Cl)cc21] | 455.1 |
| 1-21 | 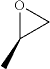 | 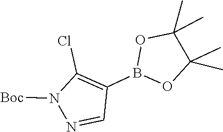 | 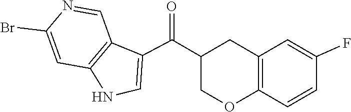 [SMILES: O=C(c1c[nH]c2cc(Br)ncc12)C1COc2ccc(F)cc2C1] | 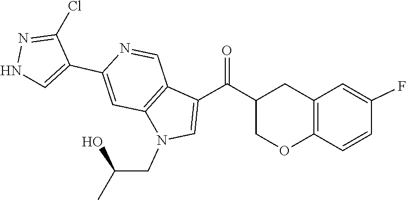 [SMILES: C[C@@H](O)Cn1cc(C(=O)C2COc3ccc(F)cc3C2)c2cnc(-c3c[nH]nc3Cl)cc21] | 455.2 |
| 1-22 | 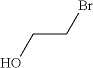 | 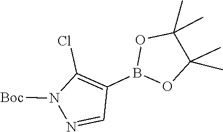 | 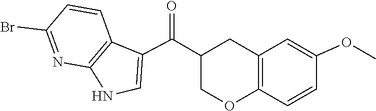 [SMILES: COc1ccc2c(c1)CC(C(=O)c1c[nH]c3nc(Br)ccc13)CO2] | 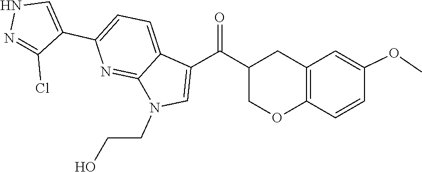 [SMILES: COc1ccc2c(c1)CC(C(=O)c1cn(CCO)c3nc(-c4c[nH]nc4Cl)ccc13)CO2] | 453.2 |
| 1-23 | 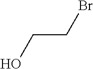 | 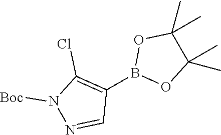 | 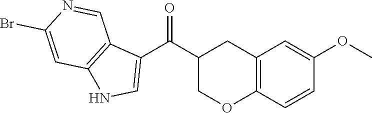 [SMILES: COc1ccc2c(c1)CC(C(=O)c1c[nH]c3cc(Br)ncc13)CO2] | 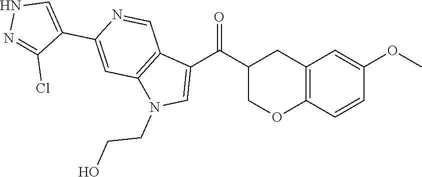 [SMILES: COc1ccc2c(c1)CC(C(=O)c1cn(CCO)c3cc(-c4c[nH]nc4Cl)ncc13)CO2] | 453.1 |
| 1-24 | 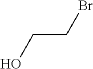 | 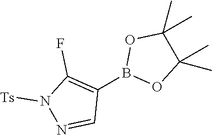 | 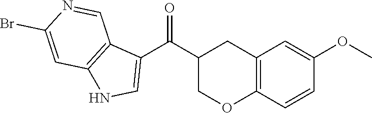 [SMILES: COc1ccc2c(c1)CC(C(=O)c1c[nH]c3cc(Br)ncc13)CO2] | 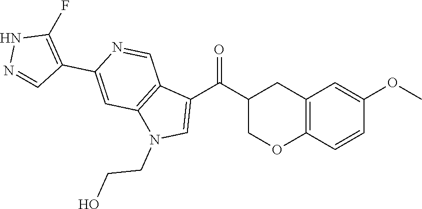 [SMILES: COc1ccc2c(c1)CC(C(=O)c1cn(CCO)c3cc(-c4cn[nH]c4F)ncc13)CO2] | 437.2 |
| 1-25 | 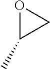 | 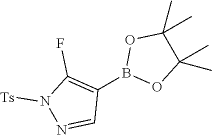 | 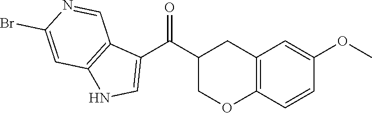 [SMILES: COc1ccc2c(c1)CC(C(=O)c1c[nH]c3cc(Br)ncc13)CO2] | 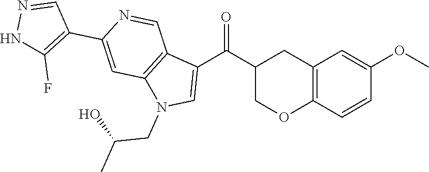 [SMILES: COc1ccc2c(c1)CC(C(=O)c1cn(C[C@H](C)O)c3cc(-c4cn[nH]c4F)ncc13)CO2] | 451.3 |
| 1-26 | 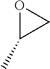 | 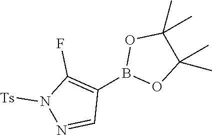 | 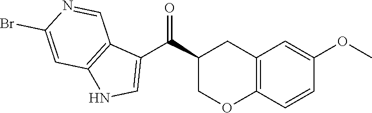 [SMILES: COc1ccc2c(c1)C[C@H](C(=O)c1c[nH]c3cc(Br)ncc13)CO2] | 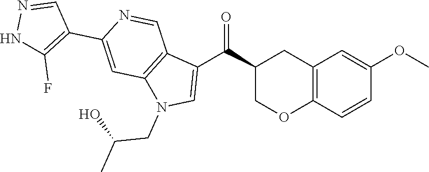 [SMILES: COc1ccc2c(c1)C[C@H](C(=O)c1cn(C[C@H](C)O)c3cc(-c4cn[nH]c4F)ncc13)CO2] | 451.2 |
| 1-27 | 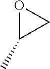 | 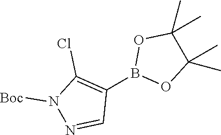 | 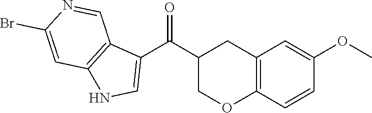 [SMILES: COc1ccc2c(c1)CC(C(=O)c1c[nH]c3cc(Br)ncc13)CO2] | 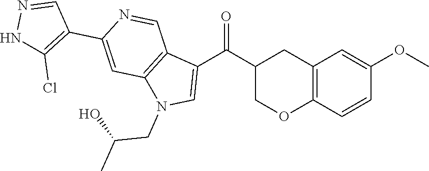 [SMILES: COc1ccc2c(c1)CC(C(=O)c1cn(C[C@H](C)O)c3cc(-c4cn[nH]c4Cl)ncc13)CO2] | 467.2 |
| 1-28 | 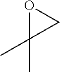 | 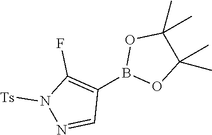 | 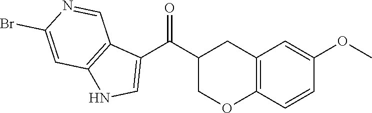 [SMILES: COc1ccc2c(c1)CC(C(=O)c1c[nH]c3cc(Br)ncc13)CO2] | 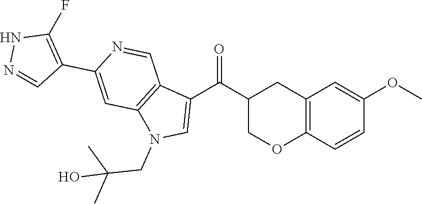 [SMILES: COc1ccc2c(c1)CC(C(=O)c1cn(CC(C)(C)O)c3cc(-c4cn[nH]c4F)ncc13)CO2] | 463.3 |
| 1-29 | 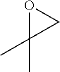 | 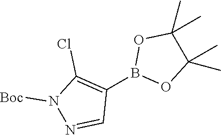 | 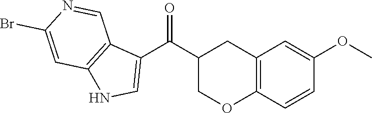 [SMILES: COc1ccc2c(c1)CC(C(=O)c1c[nH]c3cc(Br)ncc13)CO2] | 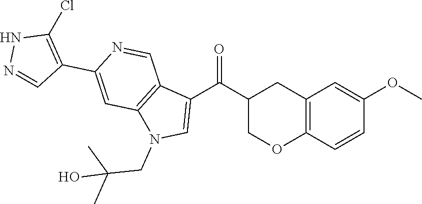 [SMILES: COc1ccc2c(c1)CC(C(=O)c1cn(CC(C)(C)O)c3cc(-c4cn[nH]c4Cl)ncc13)CO2] | 480.1 |
| 1-30 | 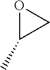 | 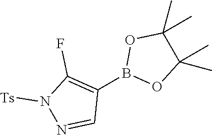 | 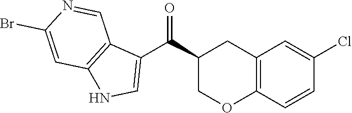 [SMILES: O=C(c1c[nH]c2cc(Br)ncc12)[C@@H]1COc2ccc(Cl)cc2C1] | 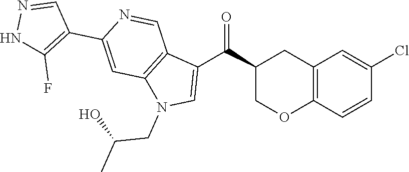 [SMILES: C[C@H](O)Cn1cc(C(=O)[C@@H]2COc3ccc(Cl)cc3C2)c2cnc(-c3cn[nH]c3F)cc21] | 455.2 |
| 1-31 | 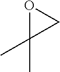 | 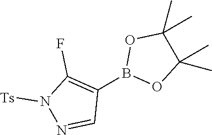 | 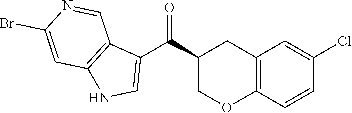 [SMILES: O=C(c1c[nH]c2cc(Br)ncc12)[C@@H]1COc2ccc(Cl)cc2C1] | 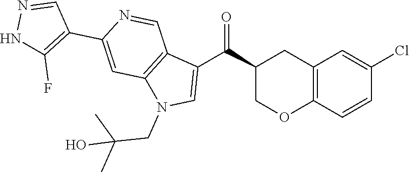 [SMILES: CC(C)(O)Cn1cc(C(=O)[C@@H]2COc3ccc(Cl)cc3C2)c2cnc(-c3cn[nH]c3F)cc21] | 469.3 |
| 1-32 | 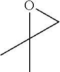 | 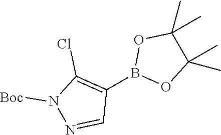 | 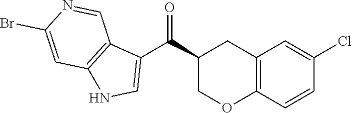 [SMILES: O=C(c1c[nH]c2cc(Br)ncc12)[C@@H]1COc2ccc(Cl)cc2C1] | 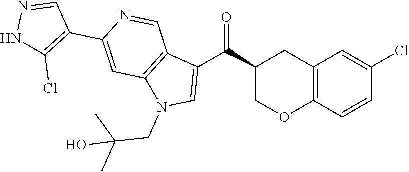 [SMILES: CC(C)(O)Cn1cc(C(=O)[C@@H]2COc3ccc(Cl)cc3C2)c2cnc(-c3cn[nH]c3Cl)cc21] | 485.2 |
| 1-33 | 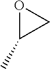 | 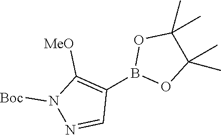 | 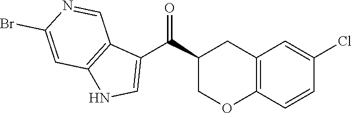 [SMILES: O=C(c1c[nH]c2cc(Br)ncc12)[C@@H]1COc2ccc(Cl)cc2C1] | 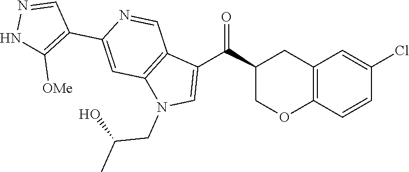 [SMILES: COc1[nH]ncc1-c1cc2c(cn1)c(C(=O)[C@@H]1COc3ccc(Cl)cc3C1)cn2C[C@H](C)O] | 467.2 |
| 1-34 | 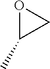 | 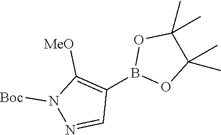 | 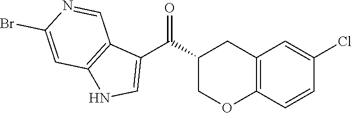 [SMILES: O=C(c1c[nH]c2cc(Br)ncc12)[C@H]1COc2ccc(Cl)cc2C1] | 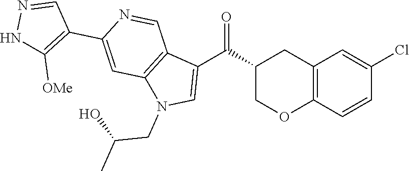 [SMILES: COc1[nH]ncc1-c1cc2c(cn1)c(C(=O)[C@H]1COc3ccc(Cl)cc3C1)cn2C[C@H](C)O] | 467.2 |
| 1-35 | 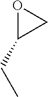 | 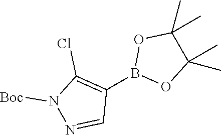 | 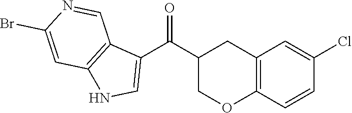 [SMILES: O=C(c1c[nH]c2cc(Br)ncc12)C1COc2ccc(Cl)cc2C1] | 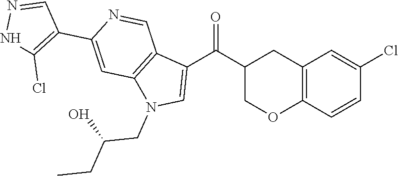 [SMILES: CC[C@H](O)Cn1cc(C(=O)C2COc3ccc(Cl)cc3C2)c2cnc(-c3cn[nH]c3Cl)cc21] | 485.2 |
| 1-36 | 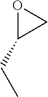 | 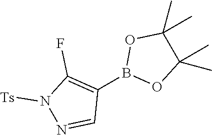 | 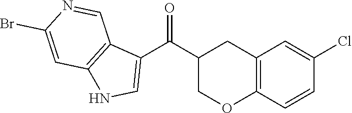 [SMILES: O=C(c1c[nH]c2cc(Br)ncc12)C1COc2ccc(Cl)cc2C1] | 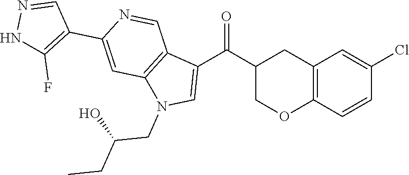 [SMILES: CC[C@H](O)Cn1cc(C(=O)C2COc3ccc(Cl)cc3C2)c2cnc(-c3cn[nH]c3F)cc21] | 469.2 |
| 1-37 | 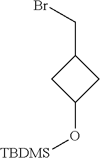 | 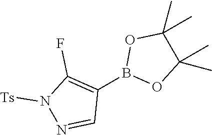 | 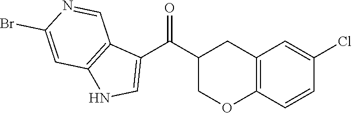 [SMILES: O=C(c1c[nH]c2cc(Br)ncc12)C1COc2ccc(Cl)cc2C1] | 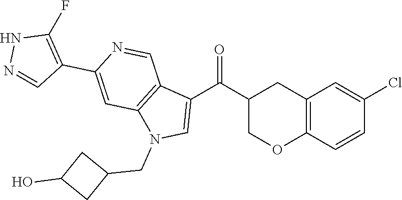 [SMILES: O=C(c1cn(CC2CC(O)C2)c2cc(-c3cn[nH]c3F)ncc12)C1COc2ccc(Cl)cc2C1] | 481.2 |
| 1-38 | 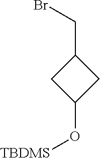 | 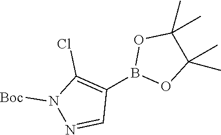 | 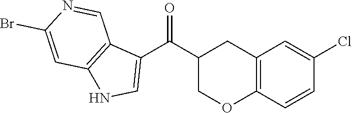 [SMILES: O=C(c1c[nH]c2cc(Br)ncc12)C1COc2ccc(Cl)cc2C1] | 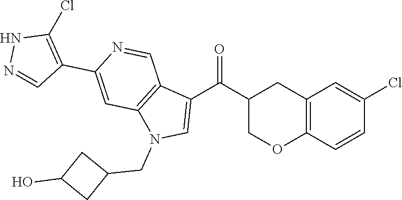 [SMILES: O=C(c1cn(CC2CC(O)C2)c2cc(-c3cn[nH]c3Cl)ncc12)C1COc2ccc(Cl)cc2C1] | 497.2 |
| 1-39 | 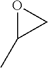 | 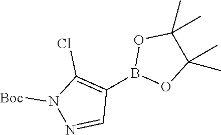 | 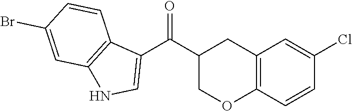 [SMILES: O=C(c1c[nH]c2cc(Br)ccc12)C1COc2ccc(Cl)cc2C1] | 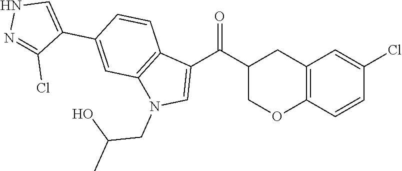 [SMILES: CC(O)Cn1cc(C(=O)C2COc3ccc(Cl)cc3C2)c2ccc(-c3c[nH]nc3Cl)cc21] | 470.1 |
| 1-40 | 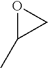 | 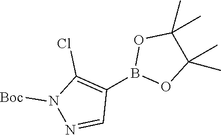 | 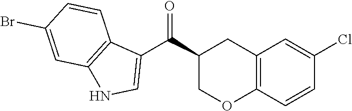 [SMILES: O=C(c1c[nH]c2cc(Br)ccc12)[C@@H]1COc2ccc(Cl)cc2C1] | 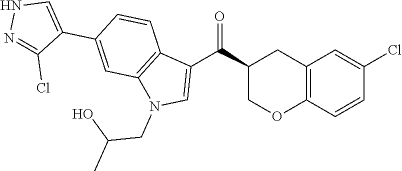 [SMILES: CC(O)Cn1cc(C(=O)[C@@H]2COc3ccc(Cl)cc3C2)c2ccc(-c3c[nH]nc3Cl)cc21] | 470.2 |
| 1-41 | 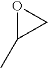 | 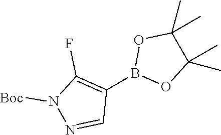 | 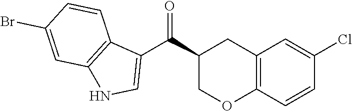 [SMILES: O=C(c1c[nH]c2cc(Br)ccc12)[C@@H]1COc2ccc(Cl)cc2C1] | 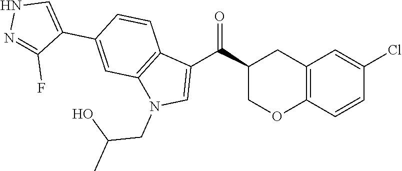 [SMILES: CC(O)Cn1cc(C(=O)[C@@H]2COc3ccc(Cl)cc3C2)c2ccc(-c3c[nH]nc3F)cc21] | 454.1 |
| 1.-42 | 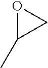 | 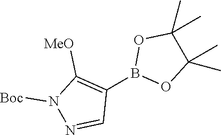 | 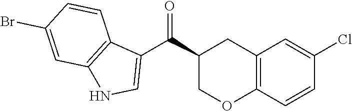 [SMILES: O=C(c1c[nH]c2cc(Br)ccc12)[C@@H]1COc2ccc(Cl)cc2C1] | 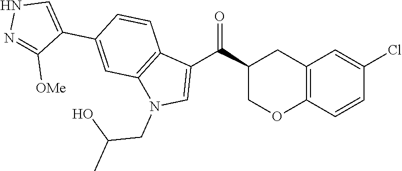 [SMILES: COc1n[nH]cc1-c1ccc2c(C(=O)[C@@H]3COc4ccc(Cl)cc4C3)cn(CC(C)O)c2c1] | 466.2 |
| 1-43 | 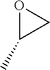 | 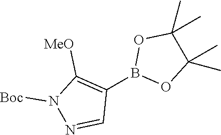 | 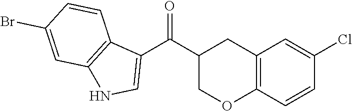 [SMILES: O=C(c1c[nH]c2cc(Br)ccc12)C1COc2ccc(Cl)cc2C1] | 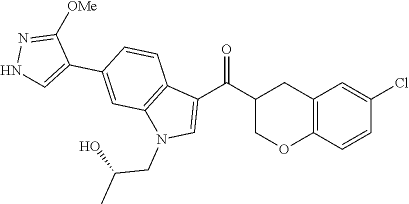 [SMILES: COc1n[nH]cc1-c1ccc2c(C(=O)C3COc4ccc(Cl)cc4C3)cn(C[C@H](C)O)c2c1] | 466.2 |
| 1-44 | 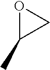 | 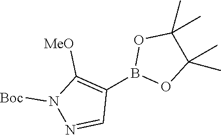 | 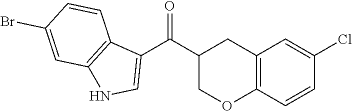 [SMILES: O=C(c1c[nH]c2cc(Br)ccc12)C1COc2ccc(Cl)cc2C1] | 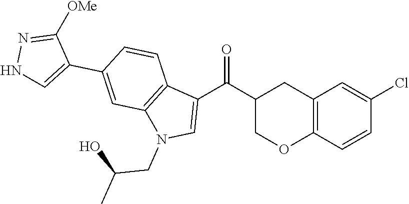 [SMILES: COc1n[nH]cc1-c1ccc2c(C(=O)C3COc4ccc(Cl)cc4C3)cn(C[C@@H](C)O)c2c1] | 466.2 |
| 1-45 | 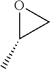 | 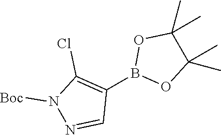 | 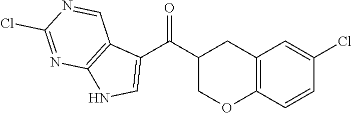 [SMILES: O=C(c1c[nH]c2nc(Cl)ncc12)C1COc2ccc(Cl)cc2C1] | 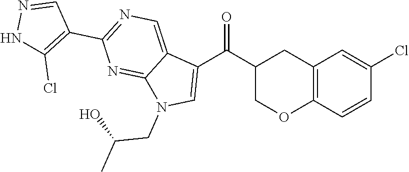 [SMILES: C[C@H](O)Cn1cc(C(=O)C2COc3ccc(Cl)cc3C2)c2cnc(-c3cn[nH]c3Cl)nc21] | 472.1 |
| 1-46 | 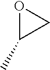 | 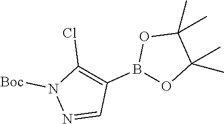 | 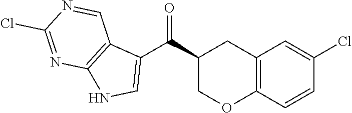 [SMILES: O=C(c1c[nH]c2nc(Cl)ncc12)[C@@H]1COc2ccc(Cl)cc2C1] | 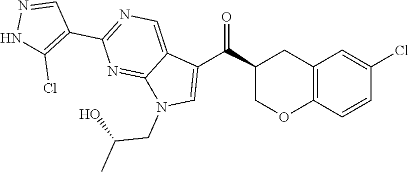 [SMILES: C[C@H](O)Cn1cc(C(=O)[C@@H]2COc3ccc(Cl)cc3C2)c2cnc(-c3cn[nH]c3Cl)nc21] | 472.1 |
| 1-47 | 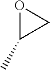 | 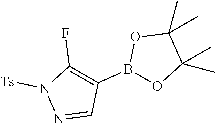 | 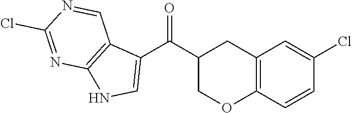 [SMILES: O=C(c1c[nH]c2nc(Cl)ncc12)C1COc2ccc(Cl)cc2C1] | 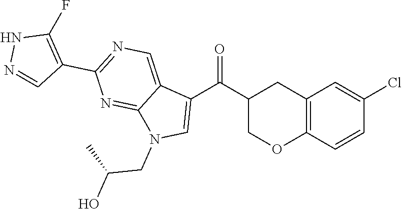 [SMILES: C[C@@H](O)Cn1cc(C(=O)C2COc3ccc(Cl)cc3C2)c2cnc(-c3cn[nH]c3F)nc21] | 455.1 |
| 1-48 | 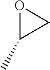 | 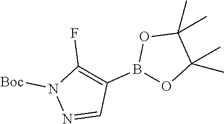 | 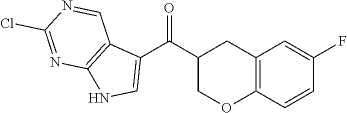 [SMILES: O=C(c1c[nH]c2nc(Cl)ncc12)C1COc2ccc(F)cc2C1] | 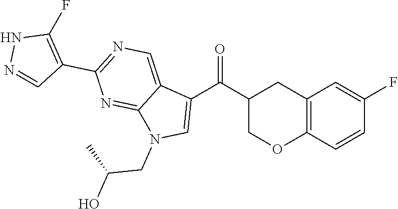 [SMILES: C[C@@H](O)Cn1cc(C(=O)C2COc3ccc(F)cc3C2)c2cnc(-c3cn[nH]c3F)nc21] | 440.1 |
| 1-49 | 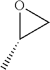 | 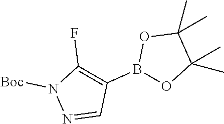 | 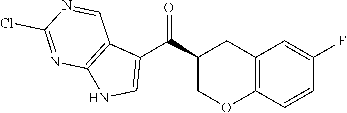 [SMILES: O=C(c1c[nH]c2nc(Cl)ncc12)[C@@H]1COc2ccc(F)cc2C1] | 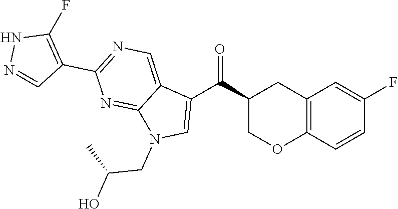 [SMILES: C[C@@H](O)Cn1cc(C(=O)[C@@H]2COc3ccc(F)cc3C2)c2cnc(-c3cn[nH]c3F)nc21] | 440.1 |
| 1-50 | 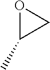 | 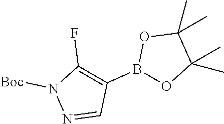 | 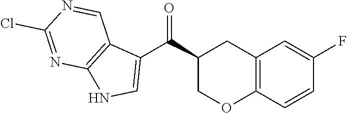 [SMILES: O=C(c1c[nH]c2nc(Cl)ncc12)[C@@H]1COc2ccc(F)cc2C1] | 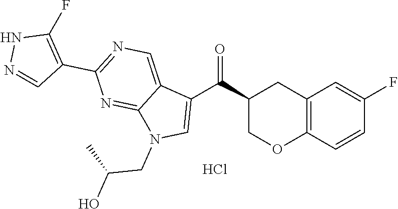 [SMILES: C[C@@H](O)Cn1cc(C(=O)[C@@H]2COc3ccc(F)cc3C2)c2cnc(-c3cn[nH]c3F)nc21 ; Cl] | 440.1 |
| 1-51 | 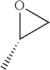 | 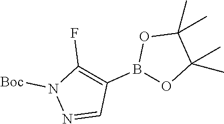 | 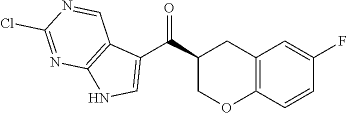 [SMILES: O=C(c1c[nH]c2nc(Cl)ncc12)[C@@H]1COc2ccc(F)cc2C1] | 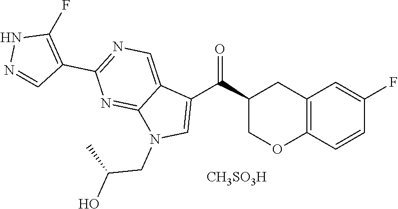 | 440.1 |
| 1-52 | 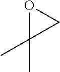 | 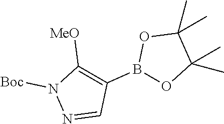 | 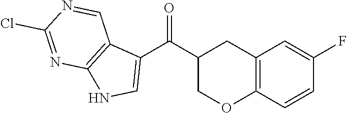 [SMILES: O=C(c1c[nH]c2nc(Cl)ncc12)C1COc2ccc(F)cc2C1] | 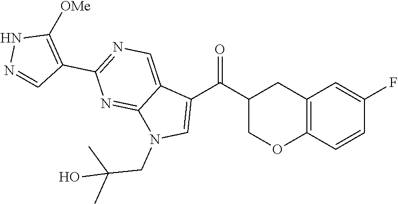 [SMILES: COc1[nH]ncc1-c1ncc2c(C(=O)C3COc4ccc(F)cc4C3)cn(CC(C)(C)O)c2n1] | 466.2 |
| 1-53 | 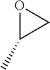 | 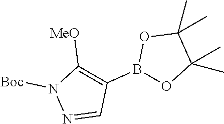 | 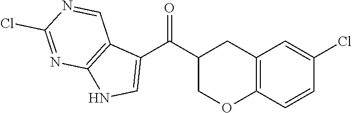 [SMILES: O=C(c1c[nH]c2nc(Cl)ncc12)C1COc2ccc(Cl)cc2C1] | 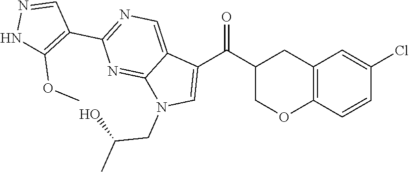 [SMILES: COc1[nH]ncc1-c1ncc2c(C(=O)C3COc4ccc(Cl)cc4C3)cn(C[C@H](C)O)c2n1] | 468.2 |
| 1-54 | 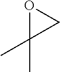 | 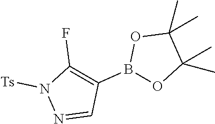 | 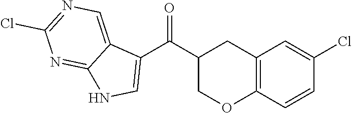 [SMILES: O=C(c1c[nH]c2nc(Cl)ncc12)C1COc2ccc(Cl)cc2C1] | 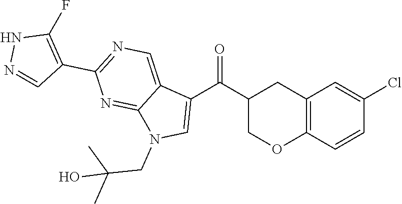 [SMILES: CC(C)(O)Cn1cc(C(=O)C2COc3ccc(Cl)cc3C2)c2cnc(-c3cn[nH]c3F)nc21] | 470.1 |
| 1-55 | 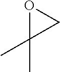 | 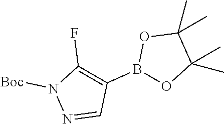 | 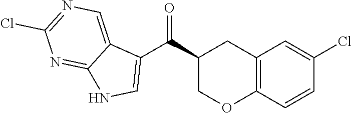 [SMILES: O=C(c1c[nH]c2nc(Cl)ncc12)[C@@H]1COc2ccc(Cl)cc2C1] | 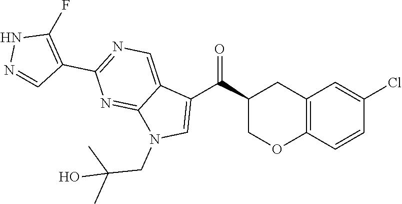 [SMILES: CC(C)(O)Cn1cc(C(=O)[C@@H]2COc3ccc(Cl)cc3C2)c2cnc(-c3cn[nH]c3F)nc21] | 470.1 |
| 1-56 | 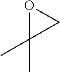 | 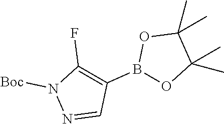 | 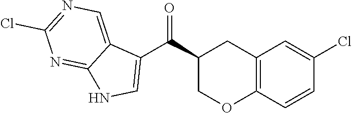 [SMILES: O=C(c1c[nH]c2nc(Cl)ncc12)[C@@H]1COc2ccc(Cl)cc2C1] | 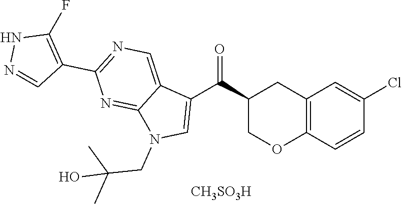 | 470.1 |
| 1-57 | 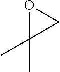 | 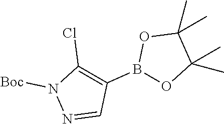 | 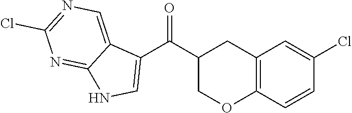 [SMILES: O=C(c1c[nH]c2nc(Cl)ncc12)C1COc2ccc(Cl)cc2C1] | 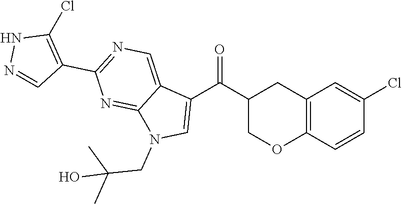 [SMILES: CC(C)(O)Cn1cc(C(=O)C2COc3ccc(Cl)cc3C2)c2cnc(-c3cn[nH]c3Cl)nc21] | 486.1 |
| 1-58 | 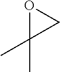 | 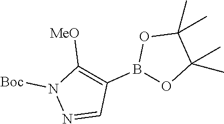 | 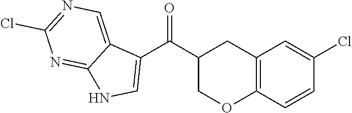 [SMILES: O=C(c1c[nH]c2nc(Cl)ncc12)C1COc2ccc(Cl)cc2C1] | 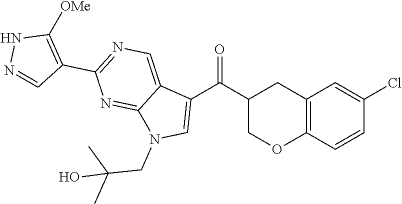 [SMILES: COc1[nH]ncc1-c1ncc2c(C(=O)C3COc4ccc(Cl)cc4C3)cn(CC(C)(C)O)c2n1] | 482.2 |
| 1-59 | 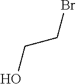 | 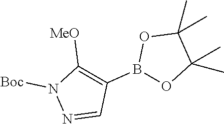 | 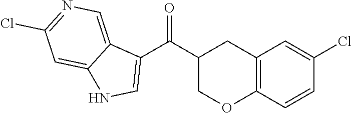 [SMILES: O=C(c1c[nH]c2cc(Cl)ncc12)C1COc2ccc(Cl)cc2C1] | 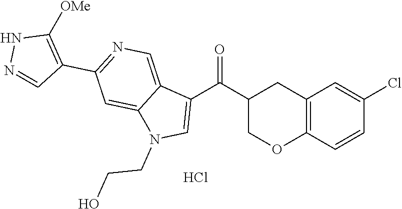 [SMILES: COc1[nH]ncc1-c1cc2c(cn1)c(C(=O)C1COc3ccc(Cl)cc3C1)cn2CCO ; Cl] | 482.2 |
| 1-60 | 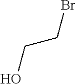 | 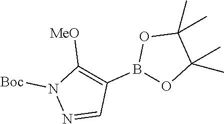 | 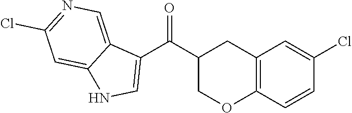 [SMILES: O=C(c1c[nH]c2cc(Cl)ncc12)C1COc2ccc(Cl)cc2C1] | 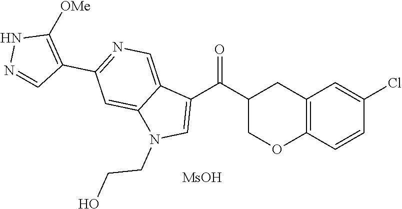 [SMILES: "MsOH" ; COc1[nH]ncc1-c1cc2c(cn1)c(C(=O)C1COc3ccc(Cl)cc3C1)cn2CCO] | 482.2 |

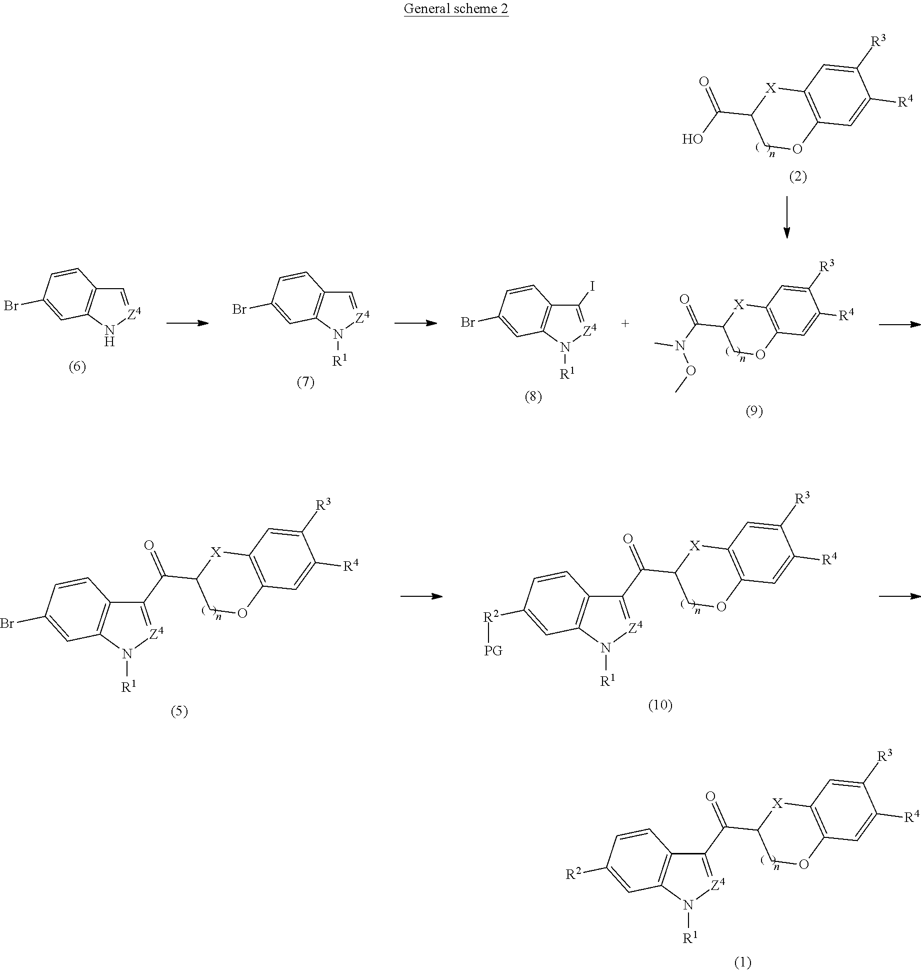

### Compound 2-1: 2,3-Dihydro-1,4-benzodioxin-3-yl-[1-(2-hydroxyethyl)-6-(1H-pyrazol-4-yl) indol-3-yl]methanone

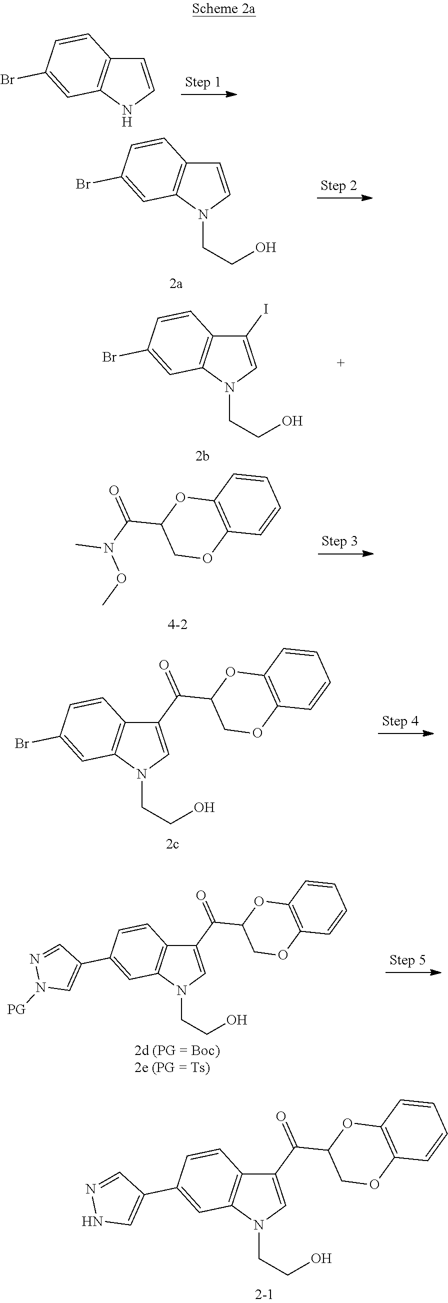

Step 1: A solution of 6-bromo-1H-indole (2.00 g, 10.20 mmol) in DMSO (20 mL) was treated with crushed potassium hydroxide (865 mg, 15.42 mmol) and stirred at room temperature for 50 minutes. A solution of 2-bromoethanol (1.53 g, 12.24 mmol) in DMSO (2.0 mL) was added dropwise (5 minutes), and stirring was continued for two days. The solution was diluted with EtOAc (400 mL), washed sequentially with water (3×400 mL) and brine (400 mL), dried (Na₂SO₄), filtered, and evaporated under reduced pressure. The residue was purified by silica gel column chromatography to provide 2a, 2-(6-bromoindol-1-yl)ethanol as an oil (1.78 g, 7.41 mmol, 73% yield). LC/MS found 240.1, +242.1 [M+H]⁺.

Step 2: A solution of 2-(6-bromoindol-1-yl)ethanol (2a, 1.78 g, 7.41 mmol) in DMF (70 mL) was cooled on an ice bath and treated with N-iodosuccinimide (NIS, 1.75 g, 7.78 mmol). The ice bath was removed, and the solution was stirred at room temperature for 90 minutes. The solution was diluted with EtOAc (500 mL), washed sequentially with water (3×500 mL) and brine (500 mL), dried (Na₂SO₄), filtered, treated with silica gel, and evaporated under reduced pressure. The material was purified by silica gel column chromatography to provide 2b, 2-(6-bromo-3-iodo-indol-1-yl)ethanol (2.17 g, 5.93 mmol, 80% yield) as a light yellow solid. LC/MS found 365.0, 367.0 [M+H]⁺.

Step 3: The 2-(6-bromo-3-iodo-indol-1-yl)ethanol (2b, 249.9 mg, 682.8 μmol) was placed under nitrogen in a 40 mL vial, then THF (9 mL) was added. The solution was cooled on a dry ice/acetonitrile bath (approximate temperature −42° C.), then treated dropwise (two minutes) with isopropylmagnesium chloride (2 M in THF, 0.72 mL). After two minutes the acetonitrile bath was replaced with an ice bath, and the sample was stirred 30 minutes. The reaction was cooled back down to −42° C., and a solution of N-methoxy-N-methyl-2,3-dihydro-1,4-benzodioxine-3-carboxamide (4-2, 183.6 mg, 822.5 μmol) in THF (2.5 mL) was added dropwise (3 minutes). The reaction was stirred for two hours, removed from the cold bath, and quenched by addition of 12% NH₄Cl (aq, 2 mL). The THF was removed under reduced pressure. The aqueous residue was diluted with additional aqueous NH₄Cl to 20 mL and extracted with EtOAc (40 mL). The organic layer was washed with brine (40 mL), dried (Na₂SO₄), and evaporated under reduced pressure. The residue was purified by silica gel column chromatography to provide the desired [6-bromo-1-(2-hydroxyethyl)indol-3-yl]-(2,3-dihydro-1,4-benzodioxin-3-yl)methanone (2c, 56.6 mg, 140.7 μmol, 21% yield) as an oil. LC/MS found 402.1, 404.1 [M+H]⁺.

Step 4: A suspension of [6-bromo-1-(2-hydroxyethyl)indol-3-yl]-(2,3-dihydro-1,4-benzodioxin-3-yl)methanone (2c, 53.4 mg, 132.8 μmol), tert-butyl 4-(4,4,5,5-tetramethyl-1,3,2-dioxaborolan-2-yl)pyrazole-1-carboxylate (60.7 mg, 206.4 μmol), PdCl₂(dppf)-CH₂Cl₂ (16.6 mg, 20.33 μmol), and potassium phosphate tribasic (88.6 mg, 417.4 μmol) in dioxane (2 mL) and water (0.24 mL) was bubbled with nitrogen 6 minutes and shaken at 65° C. overnight. The solution was evaporated under reduced pressure, and the residue was mixed with water (10 mL) and extracted with EtOAc (3×10 mL). The combined organics were dried (Na₂SO₄), filtered, and evaporated under reduced pressure to provide tert-butyl 4-[3-(2,3-dihydro-1,4-benzodioxine-3-carbonyl)-1-(2-hydroxyethyl)indol-6-yl]pyrazole-1-carboxylate (2d, 95.8 mg). The residue was used without further purification. LC/MS found 490.3 [M+H]⁺.

Step 5: A solution of crude tert-butyl 4-[3-(2,3-dihydro-1,4-benzodioxine-3-carbonyl)-1-(2-hydroxyethyl)indol-6-yl]pyrazole-1-carboxylate (2d, 95.8 mg) in DCM (1 mL) and MeOH (0.05 mL) was treated with TFA (0.5 mL) and shaken 15 minutes. The solvents were evaporated under reduced pressure. The residue was dissolved in MeOH (10 mL) and evaporated under reduced pressure. The residue was dissolved in DCM (10 mL), washed sequentially with ammonium hydroxide (1 M) and brine (10 mL each), dried (Na₂SO₄), treated with silica gel, and then evaporated under reduced pressure. The residue was purified by silica gel column chromatography to provide 2,3-dihydro-1,4-benzodioxin-3-yl-[1-(2-hydroxyethyl)-6-(1H-pyrazol-4-yl)indol-3-yl]methanone 2-1 (16.2 mg, 41.6 μmol; 31% yield for steps 4 and 5) as an orange solid. LC/MS found 390.2 [M+H]⁺.

### Compound 2-2: (6-Chlorochroman-3-yl)-[1-(2-hydroxyethyl)-6-(1H-pyrazol-4-yl)indol-3-yl]methanone

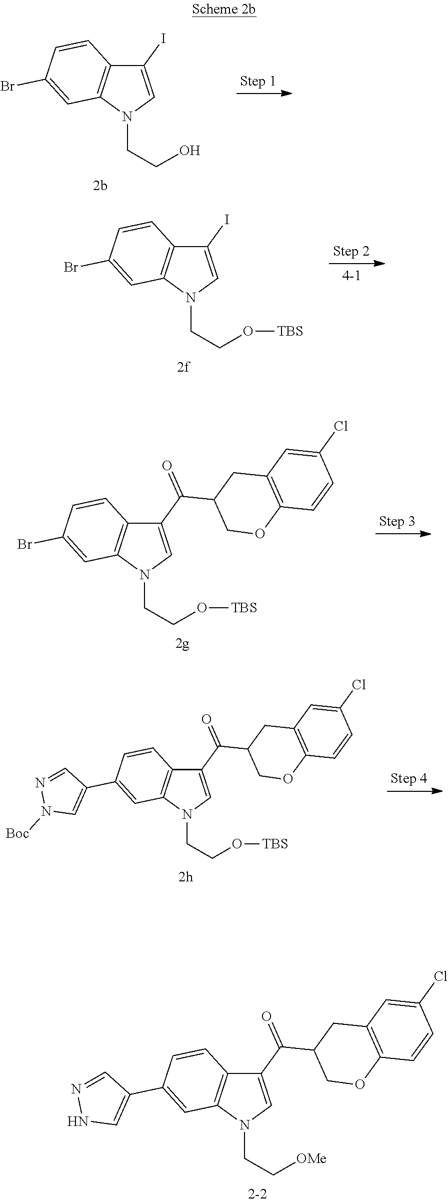

Step 1: A solution of 2-(6-bromo-3-iodo-indol-1-yl)ethanol (2b, 598.1 mg, 1.63 mmol), imidazole (225.0 mg, 3.31 mmol), and DMAP (6.7 mg, 54.84 μmol) in DMF (4 mL) was treated with TBSCl (397.6 mg, 2.64 mmol) and stirred at room temperature for 16 hours. The solution was diluted with EtOAc (40 mL), washed sequentially with water and brine (40 mL each), dried (Na₂SO₄), treated with silica gel, and evaporated under reduced pressure. The residue was purified by silica gel column chromatography to provide 2-(6-bromo-3-iodo-indol-1-yl)ethoxy-tert-butyl-dimethyl-silane (2f, 0.63 g, 1.31 mmol, 80% yield) as a clear oil. LC/MS found 479.9, 481.9 [M+H]⁺.

Step 2: A solution of 2-(6-bromo-3-iodo-indol-1-yl)ethoxy-tert-butyl-dimethyl-silane (2f, 449.2 mg, 935.3 μmol) in anhydrous THF (13 mL) was cooled on a dry ice/acetonitrile bath (approximate temperature −42° C.), then it was treated dropwise (3 minutes) with isopropylmagnesium chloride (2 M in THF, 0.65 mL). After three minutes, the acetonitrile bath was replaced with an ice bath, and the reaction mixture was stirred for 45 minutes. The reaction was cooled back down to −42° C., and a solution of 6-chloro-N-methoxy-N-methyl-chromane-3-carboxamide (4-1, 363.8 mg, 1.42 mmol) in THF (3.6 mL) was added dropwise (3 minutes). After three minutes, the dry ice bath was replaced with an ice water bath and the mixture was stirred for 2.5 hours. The reaction was quenched with water (2 mL), and the THF was evaporated under reduced pressure. The aqueous remainder was diluted with EtOAc (40 mL), washed sequentially with water and brine (40 mL each), dried (Na₂SO₄), filtered, treated with silica gel, and evaporated under reduced pressure. The residue was purified by silica gel column chromatography to provide [6-bromo-1-[2-[tert-butyl(dimethyl)silyl]oxyethyl]indol-3-yl]-(6-chlorochroman-3-yl)methanone (2g, 194.5 mg, 354.30 μmol, 38% yield) as a clear yellow oil. LC/MS found 548.3, 550.3 [M+H]⁺.

Step 3: A suspension of [6-bromo-1-[2-[tert-butyl(dimethyl)silyl]oxyethyl]indol-3-yl]-(6-chlorochroman-3-yl)methanone (2g, 63.9 mg, 116.4 μmol), tert-butyl 4-(4,4,5,5-tetramethyl-1,3,2-dioxaborolan-2-yl)pyrazole-1-carboxylate (52.4 mg, 178.1 μmol), PdCl₂(dppf)-CH₂Cl₂ (15.1 mg, 18.5 μmol), and potassium phosphate tribasic (79.0 mg, 372.2 μmol) in dioxane (2.1 mL) and water (0.25 mL) was bubbled with nitrogen for 5 minutes and shaken at 65° C. overnight. The solution was evaporated under reduced pressure, and the residue was dissolved in EtOAc (10 mL), washed sequentially with water and brine (10 mL each), dried (Na₂SO₄), filtered, and evaporated under reduced pressure to provide crude tert-butyl 4-[1-[2-[tert-butyl(dimethyl)silyl]oxyethyl]-3-(6-chlorochromane-3-carbonyl)indol-6-yl]pyrazole-1-carboxylate (2h, 99.3 mg) as a red oil. The residue was used without further purification. LC/MS found 636.2 [M+H]⁺.

Step 4: A solution of crude tert-butyl 4-[1-[2-[tert-butyl(dimethyl)silyl]oxyethyl]-3-(6-chlorochromane-3-carbonyl)indol-6-yl]pyrazole-1-carboxylate (2h, 99.3 mg) in DCM (1.0 mL) and MeOH (0.05 mL) was treated with TFA (1.0 mL) and stirred at room temperature. At about 2 hours the solvents were evaporated under reduced pressure to provide a red residue. The material was dissolved in DCM (10 mL) and washed with 1M ammonium hydroxide. The aqueous layer was extracted with DCM (10 mL), and the combined organic layers were treated with methanol to provide a clear red solution. The solution was treated with silica gel and evaporated under reduced pressure. The residue was purified by silica gel column chromatography to provide (6-chlorochroman-3-yl)-[1-(2-hydroxyethyl)-6-(1H-pyrazol-4-yl)indol-3-yl]methanone (2-2, 26.8 mg, 63.5 μmol; 55% yield for steps 3 and 4) as a brown solid. LC/MS found 422.2 [M+H]⁺.

### Compound 2-24: Chroman-3-yl-[1-(2-methoxyethyl)-6-(1H-pyrazol-4-yl)indazol-3-yl]methanone

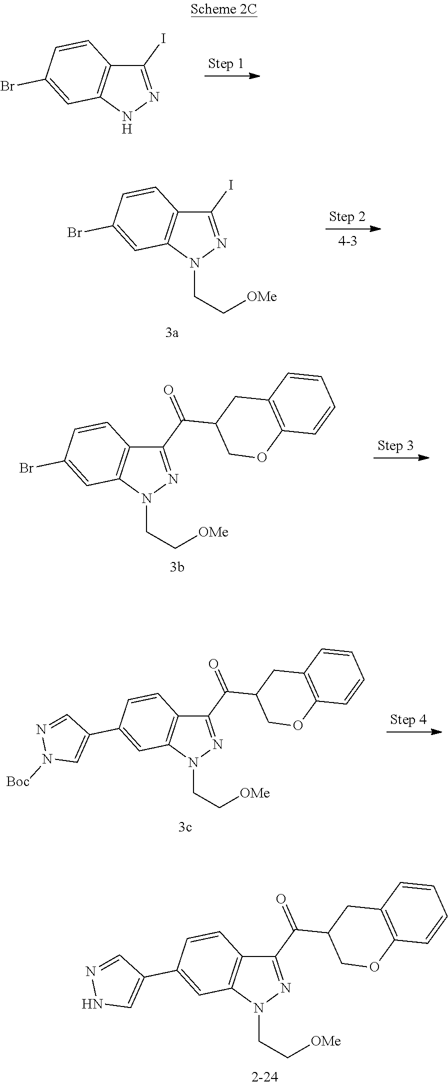 [SMILES: Brc1ccc2c(I)n[nH]c2c1 ; COCCn1nc(I)c2ccc(Br)cc21 ; COCCn1nc(C(=O)C2COc3ccccc3C2)c2ccc(Br)cc21 ; COCCn1nc(C(=O)C2COc3ccccc3C2)c2ccc(-c3cnn(C(=O)OC(C)(C)C)c3)cc21 ; COCCn1nc(C(=O)C2COc3ccccc3C2)c2ccc(-c3cn[nH]c3)cc21]

Step 1: A suspension of 6-bromo-3-iodo-1H-indazole (2.00 g, 6.19 mmol) and potassium carbonate (2.57 g, 18.58 mmol) in DMF (30 mL) was stirred at room temperature for 75 minutes. 1-Chloro-2-methoxy-ethane (0.70 mL, 7.70 mmol) was added, and the suspension was stirred at 60° C. for 9 hours. The mixture was diluted with EtOAc (225 mL), washed sequentially with water (3×225 mL) and brine (225 mL), dried (Na₂SO₄), filtered, and evaporated under reduced pressure. The residue was purified by silica gel column chromatography to provide 6-bromo-3-iodo-1-(2-methoxyethyl)indazole (3a, 1.70 g, 4.47 mmol, 72% yield) as a fluffy white solid. LC/MS found 381.0, 383.0 [M+H]⁺.

Step 2: To a solution of 6-bromo-3-iodo-1-(2-methoxyethyl)indazole (3a, 485.8 mg, 1.3 mmol) in THF (20 mL), was dropwise added of isopropylmagnesium chloride (2 M in THF, 0.76 mL) at −78° C. After being stirred for 35 minutes at −78° C., a solution of N-methoxy-N-methyl-chromane-3-carboxamide (4-3, 443.2 mg, 2.00 mmol) in THF (5 mL) was added dropwise. After 1 hour and 45 minutes the reaction was quenched by dropwise addition of water (3 mL), and the mixture was concentrated under reduced pressure. The residue was diluted with EtOAc (40 mL), and washed sequentially with aqueous ammonium chloride (12%, 20 mL) and brine (40 mL). The organic layer was dried (Na₂SO₄), decanted, and evaporated under reduced pressure. The residue was purified by silica gel column chromatography to provide [6-bromo-1-(2-methoxyethyl)indazol-3-yl]-chroman-3-yl-methanone (3b, 50.7 mg, 122.1 μmol, 10% yield) as an oil. LC/MS found 415.1, 417.1 [M+H]⁺.

Step 3: A suspension of [6-bromo-1-(2-methoxyethyl)indazol-3-yl]-chroman-3-yl-methanone (3b, 50.7 mg, 122.1 μmol), tert-butyl 4-(4,4,5,5-tetramethyl-1,3,2-dioxaborolan-2-yl)pyrazole-1-carboxylate (71.8 mg, 244.1 μmol), PdCl₂(dppf)-CH₂Cl₂ (15.2 mg, 18.61 μmol), and potassium phosphate tribasic (79.1 mg, 372.64 μmol) in dioxane (1.85 mL) and water (0.21 mL) was bubbled with nitrogen for 5 minutes and shaken at 65° C. overnight. The residue was dissolved in EtOAc (10 mL), washed with brine (10 mL), dried (Na₂SO₄), filtered, and evaporated under reduced pressure to provide impure tert-butyl 4-[3-(chromane-3-carbonyl)-1-(2-methoxyethyl)indazol-6-yl]pyrazole-1-carboxylate (3c, 119.6 mg) as a red oil. The material was used without further purification. LC/MS found 503.2 [M+H]⁺.

Step 4: A solution of crude tert-butyl 4-[3-(chromane-3-carbonyl)-1-(2-methoxyethyl)indazol-6-yl]pyrazole-1-carboxylate (3c, 119.6 mg) in DCM (1.0 mL) and MeOH (0.05 mL) was treated with TFA (0.5 mL) and shaken for 15 minutes. The solvent was evaporated under reduced pressure. The residue was dissolved in EtOAc (10 mL), washed sequentially with 1M NH₄OH and brine (10 mL each), dried (Na₂SO₄), decanted, treated with silica gel, and evaporated under reduced pressure. The residue was purified by silica gel column chromatography to provide chroman-3-yl-[1-(2-methoxyethyl)-6-(1H-pyrazol-4-yl)indazol-3-yl]methanone (2-24, 28.3 mg, 70.32 μmol; 58% yield for Steps 3 and 4) as a peach-colored solid. LC/MS 403.2 [M+H]⁺.

The following compounds 2-3 to 2-35 shown in Table 2 were prepared by methods similar to those described for the preparation of compound 2-1, 2-2, or 2-24, using the appropriate boronic acid/ester and Weinreb amide.

TABLE 2

| | R² | | | MS |
|---|---|---|---|---|
| | boronic acid or | Weinreb | | found |
| Compd | ester | amide | Structure | [M + H]⁺ |
| 2-3 | 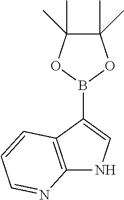 | 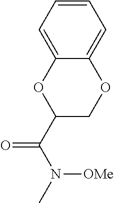 [SMILES: CON(C)C(=O)C1COc2ccccc2O1] | 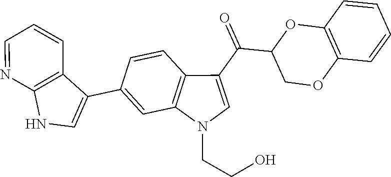 [SMILES: O=C(c1cn(CCO)c2cc(-c3c[nH]c4ncccc34)ccc12)C1COc2ccccc2O1] | 440.2 |
| 2-4 | 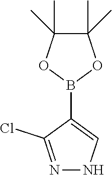 | 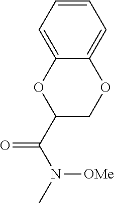 [SMILES: CON(C)C(=O)C1COc2ccccc2O1] | 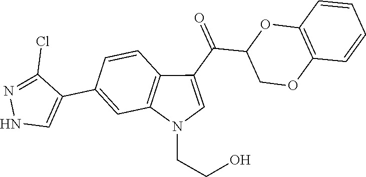 [SMILES: O=C(c1cn(CCO)c2cc(-c3c[nH]nc3Cl)ccc12)C1COc2ccccc2O1] | 424.1 |
| 2-5 | 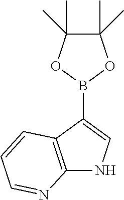 | 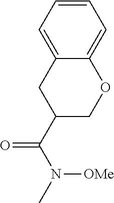 [SMILES: CON(C)C(=O)C1COc2ccccc2C1] | 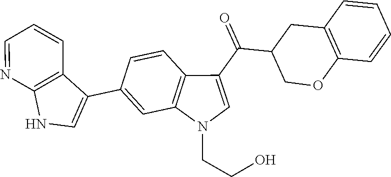 [SMILES: O=C(c1cn(CCO)c2cc(-c3c[nH]c4ncccc34)ccc12)C1COc2ccccc2C1] | 438.2 |
| 2-6 | 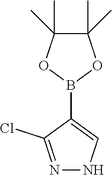 | 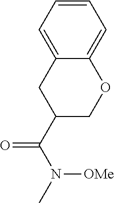 [SMILES: CON(C)C(=O)C1COc2ccccc2C1] | 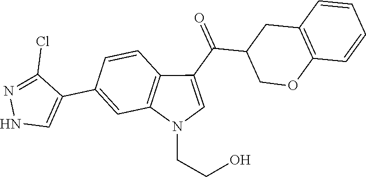 [SMILES: O=C(c1cn(CCO)c2cc(-c3c[nH]nc3Cl)ccc12)C1COc2ccccc2C1] | 422.1 |
| 2-7 | 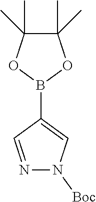 | 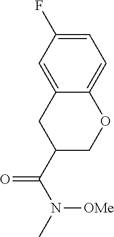 [SMILES: CON(C)C(=O)C1COc2ccc(F)cc2C1] | 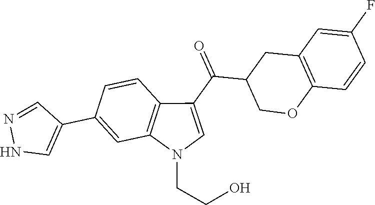 [SMILES: O=C(c1cn(CCO)c2cc(-c3cn[nH]c3)ccc12)C1COc2ccc(F)cc2C1] | 406.0 |
| 2-8 | 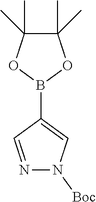 | 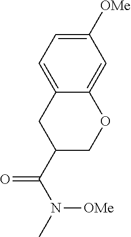 [SMILES: COc1ccc2c(c1)OCC(C(=O)N(C)OC)C2] | 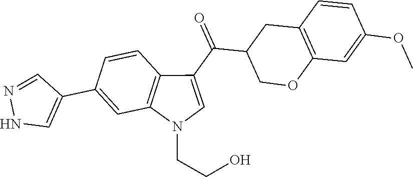 [SMILES: COc1ccc2c(c1)OCC(C(=O)c1cn(CCO)c3cc(-c4cn[nH]c4)ccc13)C2] | 418.0 |
| 2-9 | 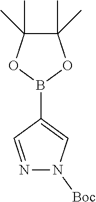 | 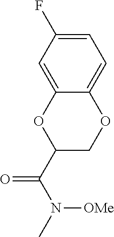 [SMILES: CON(C)C(=O)C1COc2ccc(F)cc2O1] | 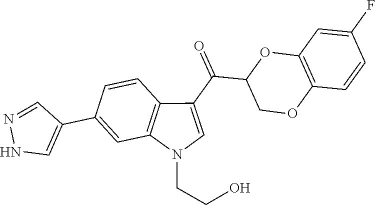 [SMILES: O=C(c1cn(CCO)c2cc(-c3cn[nH]c3)ccc12)C1COc2ccc(F)cc2O1] | 408.0 |
| 2-10 | 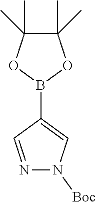 | 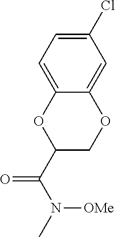 [SMILES: CON(C)C(=O)C1COc2cc(Cl)ccc2O1] | 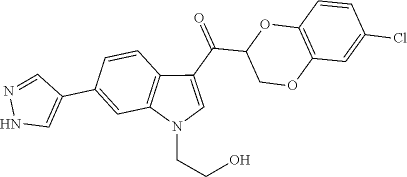 [SMILES: O=C(c1cn(CCO)c2cc(-c3cn[nH]c3)ccc12)C1COc2cc(Cl)ccc2O1] | 424.1 |
| 2-11 | 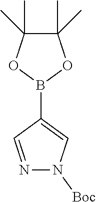 | 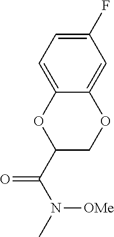 [SMILES: CON(C)C(=O)C1COc2cc(F)ccc2O1] | 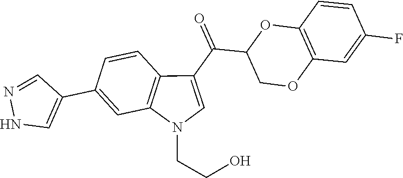 [SMILES: O=C(c1cn(CCO)c2cc(-c3cn[nH]c3)ccc12)C1COc2cc(F)ccc2O1] | 408.2 |
| 2-12 | 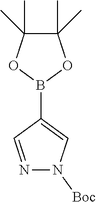 | 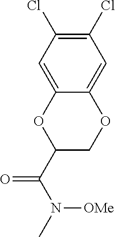 [SMILES: CON(C)C(=O)C1COc2cc(Cl)c(Cl)cc2O1] | 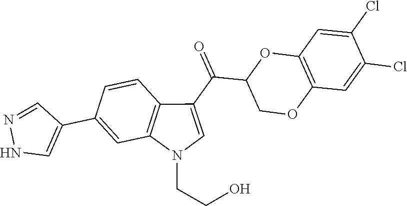 [SMILES: O=C(c1cn(CCO)c2cc(-c3cn[nH]c3)ccc12)C1COc2cc(Cl)c(Cl)cc2O1] | 458.1 |
| 2-13 | 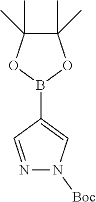 | 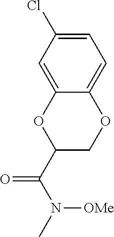 [SMILES: CON(C)C(=O)C1COc2ccc(Cl)cc2O1] | 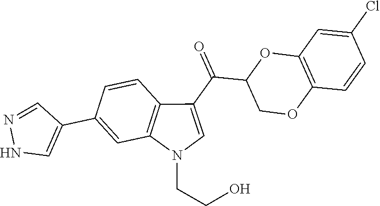 [SMILES: O=C(c1cn(CCO)c2cc(-c3cn[nH]c3)ccc12)C1COc2ccc(Cl)cc2O1] | 424.1 |
| 2-14 | 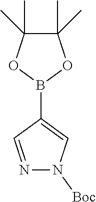 | 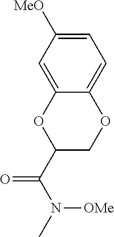 [SMILES: COc1ccc2c(c1)OC(C(=O)N(C)OC)CO2] | 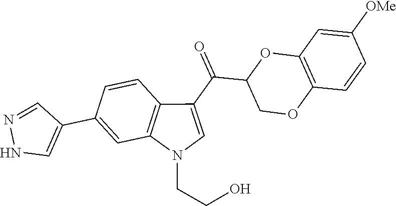 [SMILES: COc1ccc2c(c1)OC(C(=O)c1cn(CCO)c3cc(-c4cn[nH]c4)ccc13)CO2] | 420.2 |
| 2-15 | 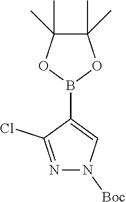 | 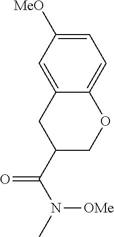 [SMILES: COc1ccc2c(c1)CC(C(=O)N(C)OC)CO2] | 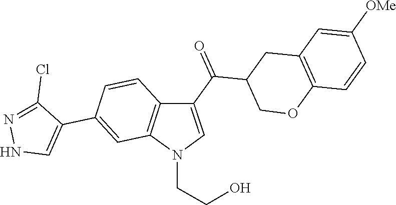 [SMILES: COc1ccc2c(c1)CC(C(=O)c1cn(CCO)c3cc(-c4c[nH]nc4Cl)ccc13)CO2] | 452.2 |
| 2-16 | 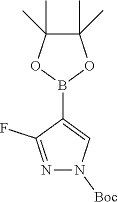 | 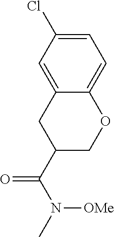 [SMILES: CON(C)C(=O)C1COc2ccc(Cl)cc2C1] | 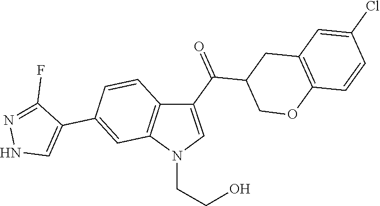 [SMILES: O=C(c1cn(CCO)c2cc(-c3c[nH]nc3F)ccc12)C1COc2ccc(Cl)cc2C1] | 440.2 |
| 2-17 | 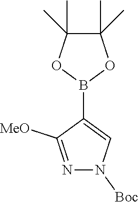 | 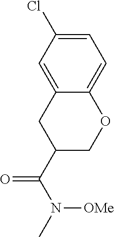 [SMILES: CON(C)C(=O)C1COc2ccc(Cl)cc2C1] | 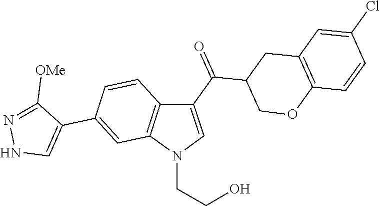 [SMILES: COc1n[nH]cc1-c1ccc2c(C(=O)C3COc4ccc(Cl)cc4C3)cn(CCO)c2c1] | 452.3 |
| 2-18 | 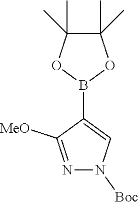 | 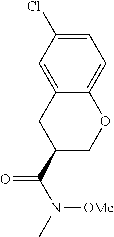 [SMILES: CON(C)C(=O)[C@@H]1COc2ccc(Cl)cc2C1] | 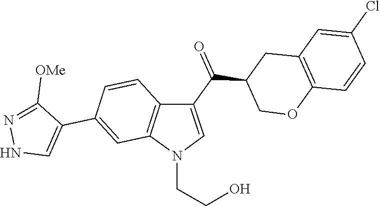 [SMILES: COc1n[nH]cc1-c1ccc2c(C(=O)[C@@H]3COc4ccc(Cl)cc4C3)cn(CCO)c2c1] | 452.2 |
| | | | ¹H NMR (400 MHz, DMSO-d6) δ ppm 12.03 | |
| | | | (br s, 1 H), 8.52 (s, 1 H), 8.10 (d, J = 8.8 Hz, 1 H), | |
| | | | 8.07 (d, J = 1.5 Hz, 1 H), 7.81 (d, J = 0.8 Hz, | |
| | | | 1 H), 7.56 (dd, J = 8.3, 1.3 Hz, 1 H), 7.24 (d, | |
| | | | J = 2.8 Hz, 1 H), 7.13 (dd, J = 8.7, 2.7 Hz, 1 H), | |
| | | | 6.82 (d, J = 8.6 Hz, 1 H), 4.98 (t, J = 5.3 Hz, 1 H), | |
| | | | 4.42-4.50 (m, 1 H), 4.27 (ddd, J = 5.8, 5.1, | |
| | | | 1.5 Hz, 2 H), 4.05 (dd, J = 10.6, 9.9 Hz, 1 H), | |
| | | | 3.93 (s, 3 H), 3.81 (q, J = 5.6 Hz, 3 H), 3.06 | |
| | | | (dd, J = 16.2, 10.1 Hz, 1 H), 2.94 (dd, J = 15.4, | |
| | | | 4.0 Hz, 1 H) | |
| 2-19 | 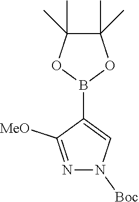 | 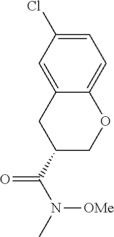 [SMILES: CON(C)C(=O)[C@H]1COc2ccc(Cl)cc2C1] | 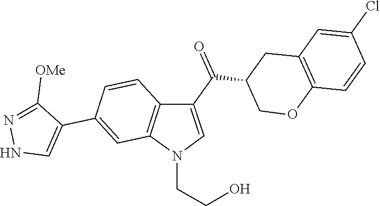 [SMILES: COc1n[nH]cc1-c1ccc2c(C(=O)[C@H]3COc4ccc(Cl)cc4C3)cn(CCO)c2c1] | 452.3 |
| 2-20 | 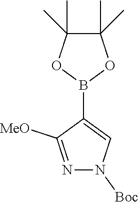 | 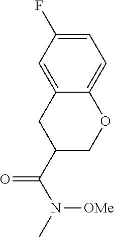 [SMILES: CON(C)C(=O)C1COc2ccc(F)cc2C1] | 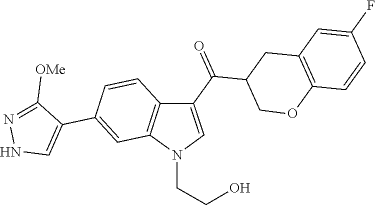 [SMILES: COc1n[nH]cc1-c1ccc2c(C(=O)C3COc4ccc(F)cc4C3)cn(CCO)c2c1] | 436.2 |
| 2-21 | 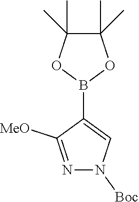 | 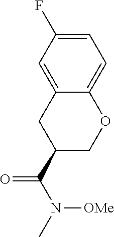 [SMILES: CON(C)C(=O)[C@@H]1COc2ccc(F)cc2C1] | 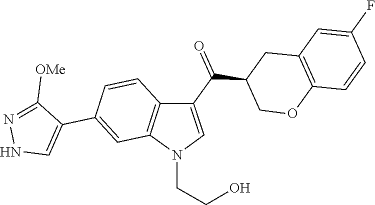 [SMILES: COc1n[nH]cc1-c1ccc2c(C(=O)[C@@H]3COc4ccc(F)cc4C3)cn(CCO)c2c1] | 436.2 |
| 2-22 | 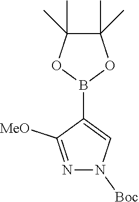 | 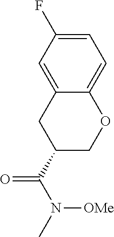 [SMILES: CON(C)C(=O)[C@H]1COc2ccc(F)cc2C1] | 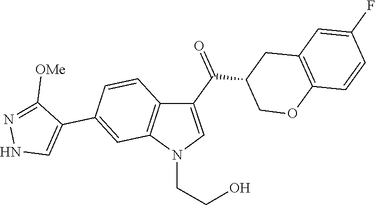 [SMILES: COc1n[nH]cc1-c1ccc2c(C(=O)[C@H]3COc4ccc(F)cc4C3)cn(CCO)c2c1] | 436.3 |
| 2-23 | 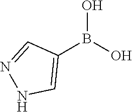 | 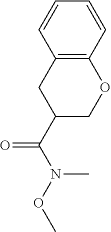 [SMILES: CON(C)C(=O)C1COc2ccccc2C1] | 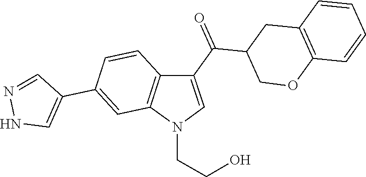 [SMILES: O=C(c1cn(CCO)c2cc(-c3cn[nH]c3)ccc12)C1COc2ccccc2C1] | 388.1 |
| 2-24 | 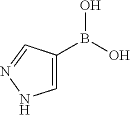 | 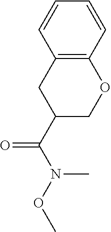 [SMILES: CON(C)C(=O)C1COc2ccccc2C1] | 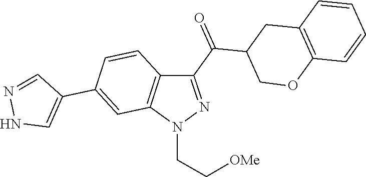 [SMILES: COCCn1nc(C(=O)C2COc3ccccc3C2)c2ccc(-c3cn[nH]c3)cc21] | 403.3 |
| 2-25 | 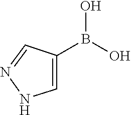 | 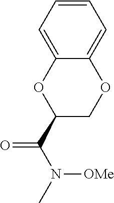 [SMILES: CON(C)C(=O)[C@@H]1COc2ccccc2O1] | 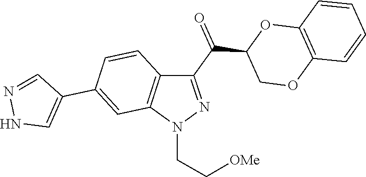 [SMILES: COCCn1nc(C(=O)[C@@H]2COc3ccccc3O2)c2ccc(-c3cn[nH]c3)cc21] | 405.2 |
| 2-26 | 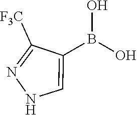 | 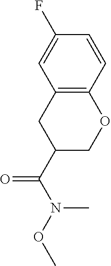 [SMILES: CON(C)C(=O)C1COc2ccc(F)cc2C1] | 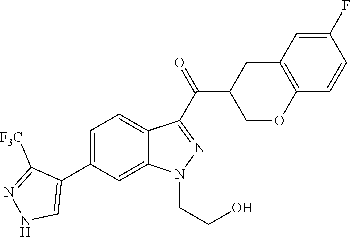 [SMILES: O=C(c1nn(CCO)c2cc(-c3c[nH]nc3C(F)(F)F)ccc12)C1COc2ccc(F)cc2C1] | 475.2 |
| 2-27 | 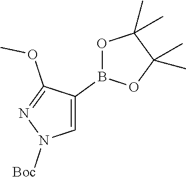 | 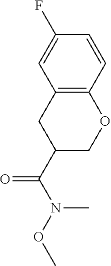 [SMILES: CON(C)C(=O)C1COc2ccc(F)cc2C1] | 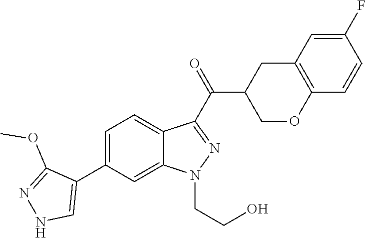 [SMILES: COc1n[nH]cc1-c1ccc2c(C(=O)C3COc4ccc(F)cc4C3)nn(CCO)c2c1] | 437.2 |
| 2-28 | 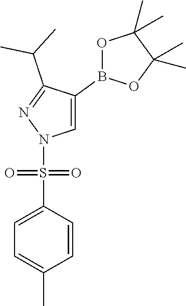 | 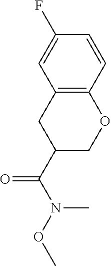 [SMILES: CON(C)C(=O)C1COc2ccc(F)cc2C1] | 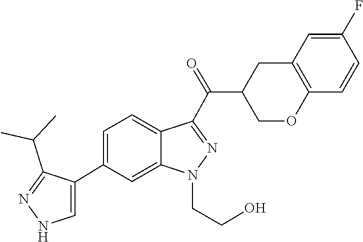 [SMILES: CC(C)c1n[nH]cc1-c1ccc2c(C(=O)C3COc4ccc(F)cc4C3)nn(CCO)c2c1] | 449.3 |
| 2-29 | 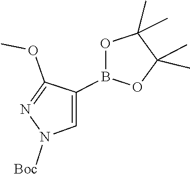 | 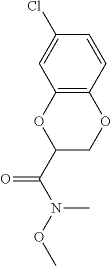 [SMILES: CON(C)C(=O)C1COc2ccc(Cl)cc2O1] | 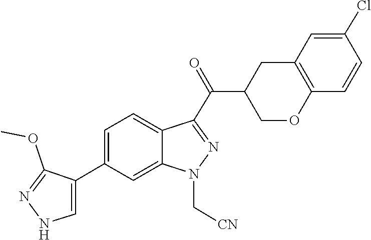 [SMILES: COc1n[nH]cc1-c1ccc2c(C(=O)C3COc4ccc(Cl)cc4C3)nn(CC#N)c2c1] | 448.4 |
| 2-30 | 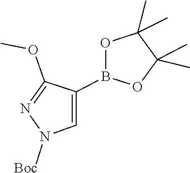 | 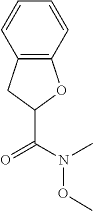 | 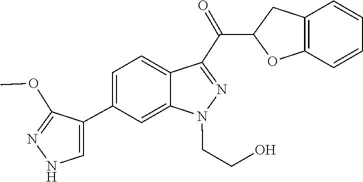 [SMILES: COc1n[nH]cc1-c1ccc2c(C(=O)C3Cc4ccccc4O3)nn(CCO)c2c1] | 405.2 |
| 2-31 | 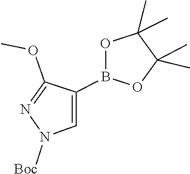 | 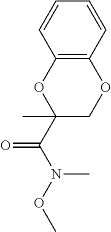 [SMILES: CON(C)C(=O)C1(C)COc2ccccc2O1] | 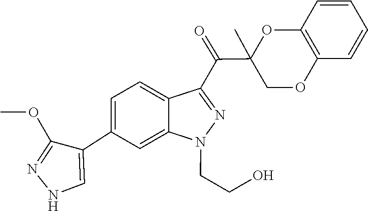 [SMILES: COc1n[nH]cc1-c1ccc2c(C(=O)C3(C)COc4ccccc4O3)nn(CCO)c2c1] | 435.3 |
| 2-32 | 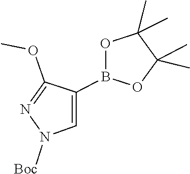 | 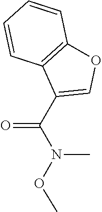 | 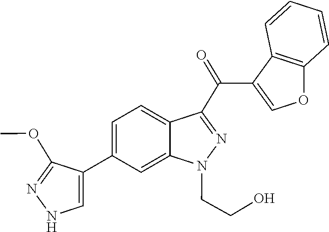 [SMILES: COc1n[nH]cc1-c1ccc2c(C(=O)c3coc4ccccc34)nn(CCO)c2c1] | 403.1 |
| 2-33 | 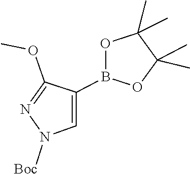 | 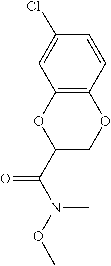 [SMILES: CON(C)C(=O)C1COc2ccc(Cl)cc2O1] | 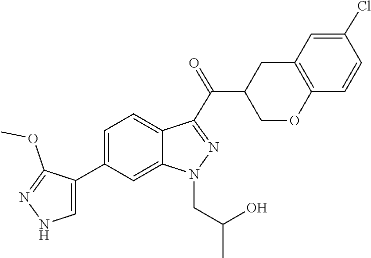 [SMILES: COc1n[nH]cc1-c1ccc2c(C(=O)C3COc4ccc(Cl)cc4C3)nn(CC(C)O)c2c1] | 467.2 |
| 2-34 | 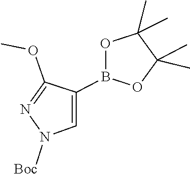 | 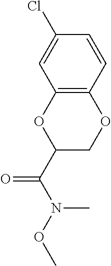 [SMILES: CON(C)C(=O)C1COc2ccc(Cl)cc2O1] | 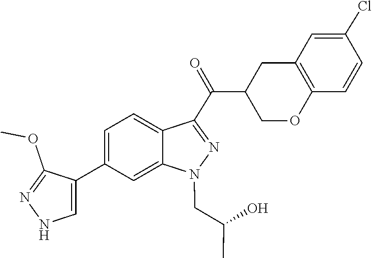 [SMILES: COc1n[nH]cc1-c1ccc2c(C(=O)C3COc4ccc(Cl)cc4C3)nn(C[C@@H](C)O)c2c1] | 467.2 |
| 2-35 | 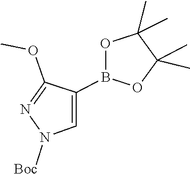 | 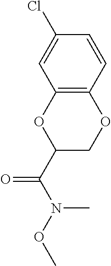 [SMILES: CON(C)C(=O)C1COc2ccc(Cl)cc2O1] | 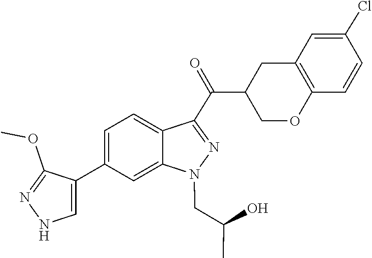 [SMILES: COc1n[nH]cc1-c1ccc2c(C(=O)C3COc4ccc(Cl)cc4C3)nn(C[C@H](C)O)c2c1] | 467.1 |

Alternatively, racemic mixtures of compounds were separated by supercritical fluid chromatography as below.

### Compound 2-18: [(3S)-6-Chlorochroman-3-yl]-[6-(5-methoxy-1H-pyrazol-4-yl)-1-(2-oxidanylethyl)indol-3-yl]methanone & Compound 2-19: [(3R)-6-Chlorochroman-3-yl]-[6-(5-methoxy-1H-pyrazol-4-yl)-1-(2-oxidanylethyl)indol-3-yl]methanone

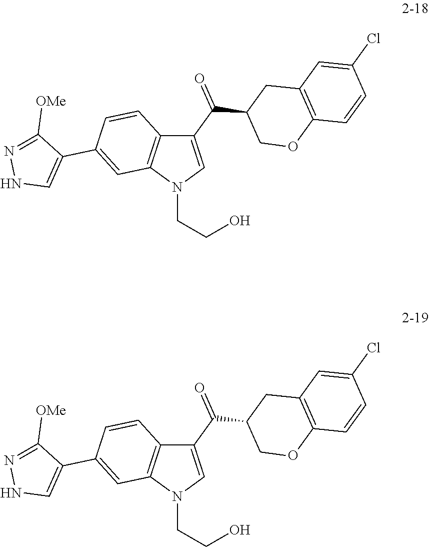 [SMILES: COc1n[nH]cc1-c1ccc2c(C(=O)[C@@H]3COc4ccc(Cl)cc4C3)cn(CCO)c2c1 ; COc1n[nH]cc1-c1ccc2c(C(=O)[C@H]3COc4ccc(Cl)cc4C3)cn(CCO)c2c1]

A sample of racemic Compound 2-17 was separated by supercritical fluid chromatography under the following conditions: Column, ChiralPak IC-H 21×250 mm; Mobile phase, 40% methanol in CO₂; Flow rate, 70 mL/min; Sample, 19.4 mg dissolved in 2.0 mL methanol and 2.0 mL dichloromethane; Injection, 0.75 mL; Detection: 220 nm.

Compound 2-18: [(3S)-6-Chlorochroman-3-yl]-[6-(5-methoxy-1H-pyrazol-4-yl)-1-(2-oxidanylethyl)indol-3-yl]methanone: first-eluting peak, 8.4 mg, 100% purity, 97.9% ee. LC/MS found 452.2 [M+H]⁺.

Compound 2-19: [(3R)-6-Chlorochroman-3-yl]-[6-(5-methoxy-1H-pyrazol-4-yl)-1-(2-oxidanylethyl)indol-3-yl]methanone: second-eluting peak, 8.7 mg, 100% purity, 96.4% ee. LC/MS found 452.2 [M+H]⁺.

### Compound 2-21: [(3S)-6-Fluorochroman-3-yl]-[1-(2-hydroxyethyl)-6-(5-methoxy-1H-pyrazol-4-yl)indol-3-yl]methanone & Compound 2-22: [(3R)-6-Fluorochroman-3-yl]-[1-(2-hydroxyethyl)-6-(5-methoxy-1H-pyrazol-4-yl)indol-3-yl]methanone

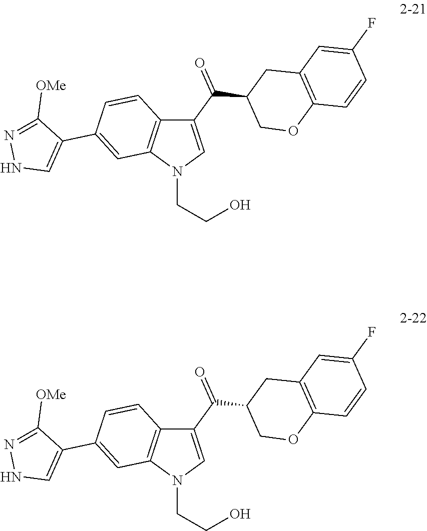 [SMILES: COc1n[nH]cc1-c1ccc2c(C(=O)[C@@H]3COc4ccc(F)cc4C3)cn(CCO)c2c1 ; COc1n[nH]cc1-c1ccc2c(C(=O)[C@H]3COc4ccc(F)cc4C3)cn(CCO)c2c1]

A sample of racemic Compound 2-20 was separated by supercritical fluid chromatography under the following conditions: Column, ChiralPak IC-H 21×250 mm; Mobile phase, 35% methanol in CO₂; Flow rate, 70 mL/min; Sample, 25.5 mg was dissolved in 2.0 mL methanol and 2.0 mL dichloromethane; Injection, 1.0 mL; Detection, 254 nm.

Compound 2-21: [(3S)-6-Fluorochroman-3-yl]-[1-(2-hydroxyethyl)-6-(5-methoxy-1H-pyrazol-4-yl)indol-3-yl]methanone: first-eluting peak, 10.9 mg, 100% purity, 98.5% ee. LC/MS found 436.2 [M+H]⁺.

Compound 2-22: Compound 2-22: [(3R)-6-Fluorochroman-3-yl]-[1-(2-hydroxyethyl)-6-(5-methoxy-1H-pyrazol-4-yl)indol-3-yl]methanone: second-eluting peak, 10.8 mg, 100% purity, 97.7% ee. LC/MS found 436.2 [M+H]⁺.

### Compound 3-1: Chroman-3-yl-[1-(2-methoxyethyl)-6-(1H-pyrazol-4-yl)indazol-3-yl]methanone

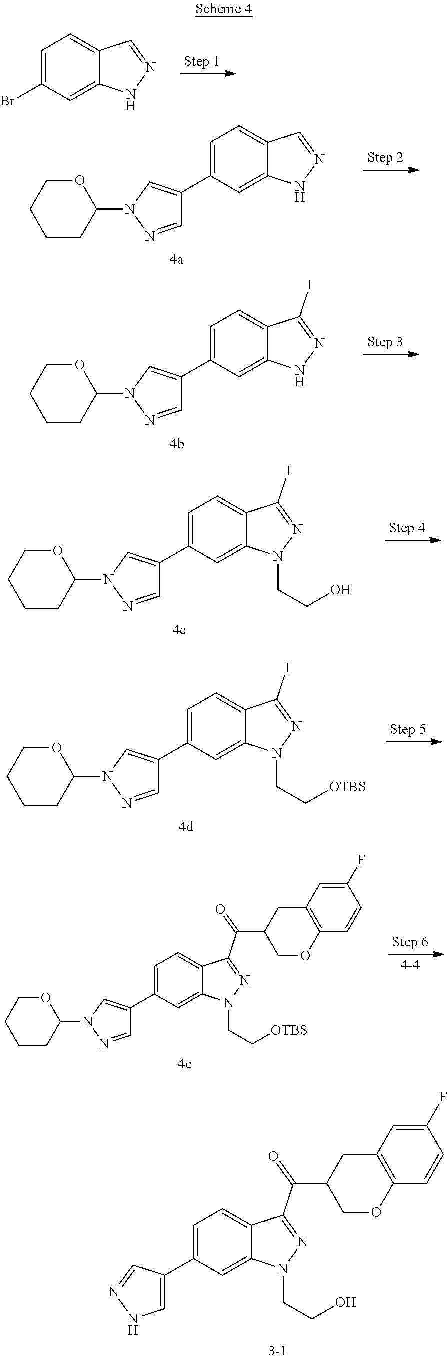

Step 1: A mixture of 6-bromo-1H-indazole (985 mg, 5.00 mmol) and 1-tetrahydropyran-2-yl-4-(4,4,5,5-tetramethyl-1,3,2-dioxaborolan-2-yl)pyrazole (2.09 g, 7.50 mmol) tris(dibenzylideneacetone)dipalladium, tri-t-butylphosphonium tetrafluoroborate (4 mol %), and Na₂CO₃ (3 eq.) in 1:1 mixture of ethanol/water (10 mL) was degassed via sparging with N for 5 minutes and heated to 100° C. with stirring overnight. The mixture was cooled to room temperature, poured over water, and extracted with EtOAc. The combined organic layers were washed with brine, dried over MgSO₄, filtered, and then concentrated. The residue was purified by silica gel column chromatography with 50-100% EtOAc in heptanes to provide 4a, 6-(1-(tetrahydro-2H-pyran-2-yl)-1H-pyrazol-4-yl)-1H-indazole as a white foamy solid in 90% yield (4a, 1.21 g, 4.51 mmol). LC/MS found 269.2 [M+H]⁺.

Step 2: To a solution of 6-(1-(tetrahydro-2H-pyran-2-yl)-1H-pyrazol-4-yl)-1H-indazole (4a, 1.01 g, 3.76 mmol) in DMF (10 mL) was added potassium carbonate (1.04 g, 7.53 mmol) followed by N-iodosuccinimide (1.02 g, 4.52 mmol). The mixture was stirred at room temperature overnight and poured into 10% aqueous NaHSO₃. The mixture was extracted with EtOAc, and the combined organic layers were washed with water, washed with brine, dried over Na₂SO₄, filtered, and then concentrated to provide 3-iodo-6-(1-(tetrahydro-2H-pyran-2-yl)-1H-pyrazol-4-yl)-1H-indazole as a white solid in 86% yield (4b, 1.27 g, 3.22 mmol). LC/MS found 395.1 [M+H]⁺. The crude product was used without further purification.

Step 3: To a solution of 3-iodo-6-(1-(tetrahydro-2H-pyran-2-yl)-1H-pyrazol-4-yl)-1H-indazole (4b, 1.27 g, 3.22 mmol) in acetonitrile (32 mL) was added potassium carbonate (888 mg, 6.43 mmol) followed by ethylene oxide (2.9 M in THF, 2.2 mL, 6.4 mmol). The mixture was heated to 80° C. with stirring overnight. The mixture was cooled to room temperature, poured over water, and extracted with EtOAc. The combined organic layers were washed with brine, dried over Na₂SO₄, filtered, and then concentrated. The residue was purified by silica gel column chromatography using a gradient of 50-100% EtOAc in heptanes to provide 4c, 2-(3-iodo-6-(1-(tetrahydro-2H-pyran-2-yl)-1H-pyrazol-4-yl)-1H-indazol-1-yl)ethan-1-ol as a solid in 32% yield (450 mg, 1.03 mmol). LC/MS found 439.1 [M+H]⁺.

Step 4: To a solution of 2-(3-iodo-6-(1-(tetrahydro-2H-pyran-2-yl)-1H-pyrazol-4-yl)-1H-indazol-1-yl)ethan-1-ol (4c, 437 mg, 1.00 mmol) and imidazole (140 mg, 2.06 mmol) in DMF (4 mL) cooled to 0 C was added tert-butyldimethylchlorosilane (170 mg, 1.13 mmol). The mixture was warmed to room temperature and stirred overnight. The mixture was poured over water and extracted with 1:1 EtOAc/hexanes. The combined organic layers were washed with water (3×), washed with brine, dried over Na₂SO₄, filtered, and concentrated. The residue was purified by silica gel column chromatography using a gradient of 1-10% EtOAc in hexanes to provide 1-(2-((tert-butyldimethylsilyl)oxy)ethyl)-3-iodo-6-(1-(tetrahydro-2H-pyran-2-yl)-1H-pyrazol-4-yl)-1H-indazole as a white solid in 88% yield (4d, 487 mg, 0.88 mmol). LC/MS found 553.3 [M+H]⁺.

Step 5: To a solution of 1-(2-((tert-butyldimethylsilyl)oxy)ethyl)-3-iodo-6-(1-(tetrahydro-2H-pyran-2-yl)-1H-pyrazol-4-yl)-1H-indazole (4d, 85 mg, 0.15 mmol) in dry THF (2 mL) cooled to −40° C. was added dropwise iso-propylmagnesium chloride (2.0 M in THF, 0.11 mL, 0.22 mmol). After 5 minutes the mixture was warmed to 0° C. and allowed to stir at this temperature for 45 minutes. The mixture was cooled back down to −40° C., and a solution of 6-fluoranyl-N-methoxy-N-methyl-chromane-3-carboxamide (62 mg, 0.26 mmol) in dry THF (1 mL) was added dropwise. The mixture was warmed back up to 0° C. and allowed to stir for 2 hours. The reaction was quenched by the addition of water and extracted with EtOAc. The combined organic layers were washed with 10% aqueous NH₄Cl, washed with brine, dried over Na₂SO₄, filtered, and concentrated to dryness. The residue was purified by silica gel column chromatography using a gradient of 1-10% EtOAc in heptanes to provide (1-(2-((tert-butyldimethylsilyl)oxy)ethyl)-6-(1-(tetrahydro-2H-pyran-2-yl)-1H-pyrazol-4-yl)-1H-indazol-3-yl)(6-fluorochroman-3-yl)methanone as a white solid in 38% yield (4e, 35 mg, 59 μmol).

Step 6: To a solution of (1-(2-((tert-butyldimethylsilyl)oxy)ethyl)-6-(1-(tetrahydro-2H-pyran-2-yl)-1H-pyrazol-4-yl)-1H-indazol-3-yl)(6-fluorochroman-3-yl)methanone (4e, 35 mg, 59 mol) in 1:1 mixture of THF/water (2 mL) was added p-toluenesulfonic acid monohydrate (11 mg), and the mixture was heated to 60° C. with stirring overnight. The mixture was cooled to room temperature, poured over water, and extracted with EtOAc. The combined organic layers were washed with brine, dried over Na₂SO₄, filtered, and concentrated. The residue was purified by silica gel column chromatography using a gradient of 1-10% CH₃OH in CH₂Cl₂ to provide (6-fluorochroman-3-yl)(1-(2-hydroxyethyl)-6-(1H-pyrazol-4-yl)-1H-indazol-3-yl)methanone as a white solid in 24% yield (3-1, 5.7 mg, 14 μmol). LC/MS found 407.1 [M+H]⁺.

The following compounds 3-2 to 3-4 shown in Table 3 were prepared by methods similar to those described for the preparation of compound 3-1, using the appropriate Weinreb amide and the appropriate R² boronic ester.

TABLE 3

| | | | | LC/MS |
|---|---|---|---|---|
| | R² boronic | Weinreb | | found |
| Compound | ester | amide | Structure | [M + H]⁺ |
| 3-2 | 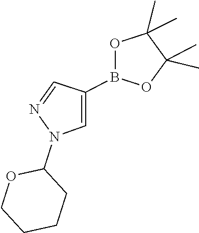 | 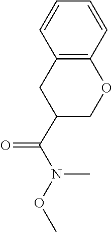 [SMILES: CON(C)C(=O)C1COc2ccccc2C1] | 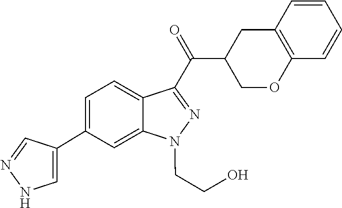 [SMILES: O=C(c1nn(CCO)c2cc(-c3cn[nH]c3)ccc12)C1COc2ccccc2C1] | 389.1 |
| 3-3 | 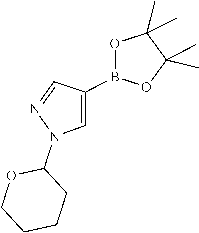 | 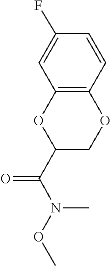 [SMILES: CON(C)C(=O)C1COc2ccc(F)cc2O1] | 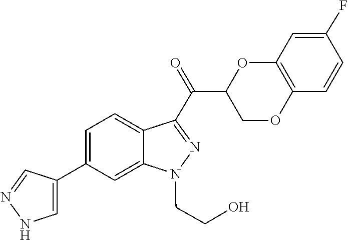 [SMILES: O=C(c1nn(CCO)c2cc(-c3cn[nH]c3)ccc12)C1COc2ccc(F)cc2O1] | 409.2 |
| 3-4 | 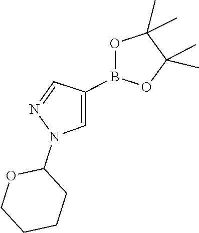 | 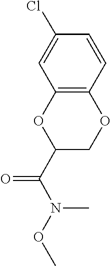 [SMILES: CON(C)C(=O)C1COc2ccc(Cl)cc2O1] | 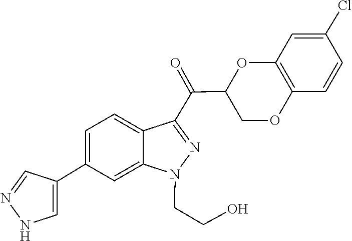 [SMILES: O=C(c1nn(CCO)c2cc(-c3cn[nH]c3)ccc12)C1COc2ccc(Cl)cc2O1] | 425.1 |

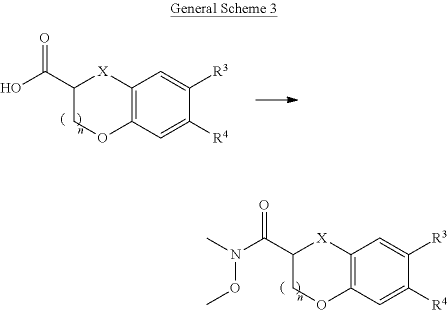

### Intermediate 4-1: 6-Chloro-N-methoxy-N-methyl-chromane-3-carboxamide

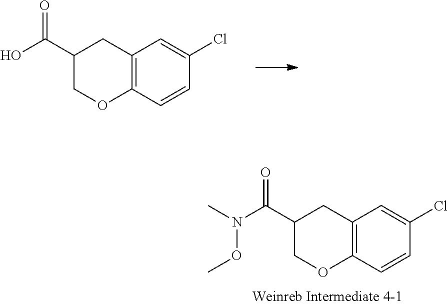 [SMILES: O=C(O)C1COc2ccc(Cl)cc2C1 ; CON(C)C(=O)C1COc2ccc(Cl)cc2C1]

A suspension of 6-chlorochromane-3-carboxylic acid (964.5 mg, 4.54 mmol), N,O-dimethyl hydroxylamine hydrochloride (665.3 mg, 6.82 mmol), and 1-[bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide (HATU) (2.15 g, 5.67 mmol) in DMF (33 mL) was treated with N,N-diisopropylethylamine (18.4 mmol, 3.2 mL). The solution was stirred at ambient temperature overnight. The solution was diluted with EtOAc (300 ml), washed successively with water (3×300 mL) and brine (300 mL), dried (Na₂SO₄), filtered, and evaporated under reduced pressure. The material was purified by silica gel column chromatography to provide 6-chloro-N-methoxy-N-methyl-chromane-3-carboxamide (Weinreb Intermediate 4-1; 1.06 g, 4.15 mmol, 91% yield) as a clear, colorless oil. LC/MS found 256.0 [M+H]⁺.

The following Weinreb amide Intermediates 4-2 through 4-23 shown in Table 4 were prepared by methods similar to those described for the preparation of Intermediate 4-1, using the appropriate carboxylic acid.

TABLE 4

| | | | LC/MS |
|---|---|---|---|
| Intermediate | Carboxylic acid | Weinreb Intermediate | [M + H]⁺ |
| 4-2 | 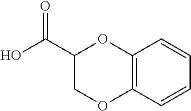 [SMILES: O=C(O)C1COc2ccccc2O1] | 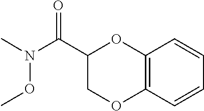 [SMILES: CON(C)C(=O)C1COc2ccccc2O1] | 224.1 |
| 4-3 | 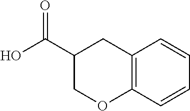 [SMILES: O=C(O)C1COc2ccccc2C1] | 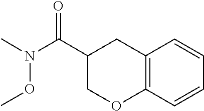 [SMILES: CON(C)C(=O)C1COc2ccccc2C1] | 222.2 |
| 4-4 | 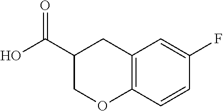 [SMILES: O=C(O)C1COc2ccc(F)cc2C1] | 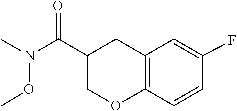 [SMILES: CON(C)C(=O)C1COc2ccc(F)cc2C1] | 239.9 |
| 4-5 | 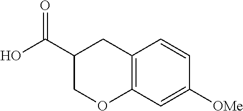 [SMILES: COc1ccc2c(c1)OCC(C(=O)O)C2] | 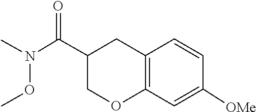 [SMILES: COc1ccc2c(c1)OCC(C(=O)N(C)OC)C2] | 251.9 |
| 4-6 | 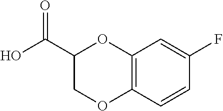 [SMILES: O=C(O)C1COc2ccc(F)cc2O1] | 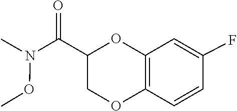 [SMILES: CON(C)C(=O)C1COc2ccc(F)cc2O1] | 242.1 |
| 4-7 | 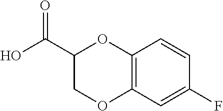 [SMILES: O=C(O)C1COc2cc(F)ccc2O1] | 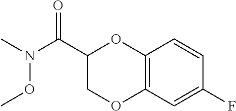 [SMILES: CON(C)C(=O)C1COc2cc(F)ccc2O1] | 242.3 |
| 4-8 | 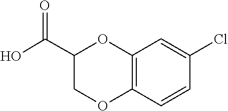 [SMILES: O=C(O)C1COc2ccc(Cl)cc2O1] | 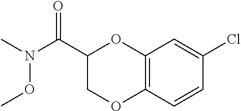 [SMILES: CON(C)C(=O)C1COc2ccc(Cl)cc2O1] | 258.1 |
| 4-9 | 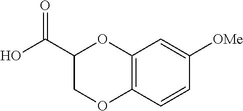 [SMILES: COc1ccc2c(c1)OC(C(=O)O)CO2] | 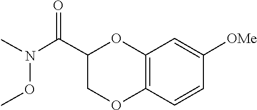 [SMILES: COc1ccc2c(c1)OC(C(=O)N(C)OC)CO2] | 254.1 |
| 4-10 | 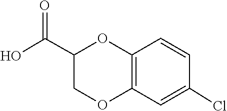 [SMILES: O=C(O)C1COc2cc(Cl)ccc2O1] | 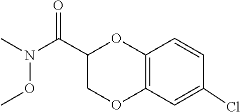 [SMILES: CON(C)C(=O)C1COc2cc(Cl)ccc2O1] | 258.0 |
| 4-11 | 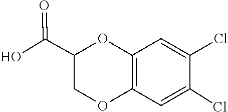 [SMILES: O=C(O)C1COc2cc(Cl)c(Cl)cc2O1] | 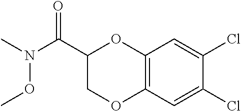 [SMILES: CON(C)C(=O)C1COc2cc(Cl)c(Cl)cc2O1] | 292.1 |
| 4-12 | 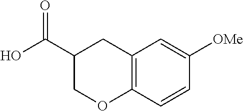 [SMILES: COc1ccc2c(c1)CC(C(=O)O)CO2] | 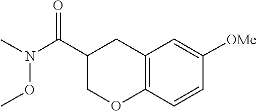 [SMILES: COc1ccc2c(c1)CC(C(=O)N(C)OC)CO2] | 252.1 |
| 4-13 | 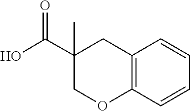 | 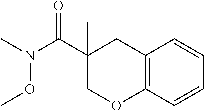 [SMILES: CON(C)C(=O)C1(C)COc2ccccc2C1] | 236.2 |
| 4-14 | 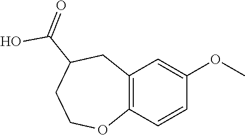 [SMILES: COc1ccc2c(c1)CC(C(=O)O)CCO2] | 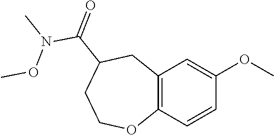 | 266.1 |
| 4-15 | 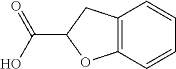 [SMILES: O=C(O)C1Cc2ccccc2O1] | 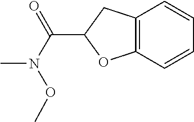 | 208.1 |
| 4-16 | 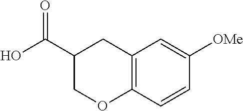 [SMILES: COc1ccc2c(c1)CC(C(=O)O)CO2] | 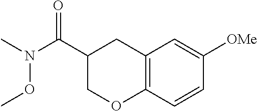 [SMILES: COc1ccc2c(c1)CC(C(=O)N(C)OC)CO2] | 251.2 |
| 4-17 | 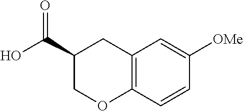 [SMILES: COc1ccc2c(c1)C[C@H](C(=O)O)CO2] | 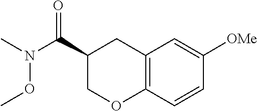 [SMILES: COc1ccc2c(c1)C[C@H](C(=O)N(C)OC)CO2] | 251.1 |
| 4-18 | 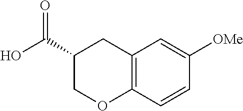 [SMILES: COc1ccc2c(c1)C[C@@H](C(=O)O)CO2] | 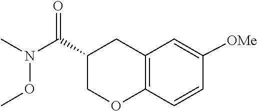 [SMILES: COc1ccc2c(c1)C[C@@H](C(=O)N(C)OC)CO2] | 252.1 |
| 4-19 | 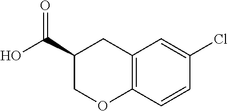 [SMILES: O=C(O)[C@@H]1COc2ccc(Cl)cc2C1] | 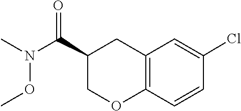 [SMILES: CON(C)C(=O)[C@@H]1COc2ccc(Cl)cc2C1] | 256.1 |
| 4-20 | 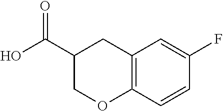 [SMILES: O=C(O)C1COc2ccc(F)cc2C1] | 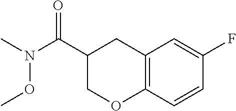 [SMILES: CON(C)C(=O)C1COc2ccc(F)cc2C1] | 240.2 |
| 4-21 | 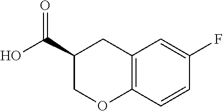 [SMILES: O=C(O)[C@@H]1COc2ccc(F)cc2C1] | 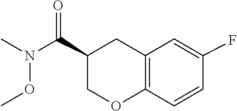 [SMILES: CON(C)C(=O)[C@@H]1COc2ccc(F)cc2C1] | 240.2 |
| 4-22 | 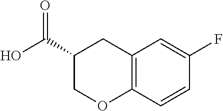 [SMILES: O=C(O)[C@H]1COc2ccc(F)cc2C1] | 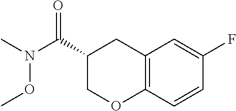 [SMILES: CON(C)C(=O)[C@H]1COc2ccc(F)cc2C1] | 240.1 |
| 4-23 | 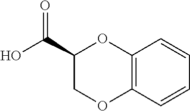 [SMILES: O=C(O)[C@@H]1COc2ccccc2O1] | 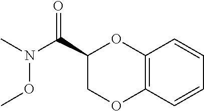 [SMILES: CON(C)C(=O)[C@@H]1COc2ccccc2O1] | 224.2 |

### Example 2: ROCK Inhibition Assay

Kinase IC₅₀ was determined through an in vitro assay based on LANCE Ultra TR-FRET (Time-Resolved Fluorescence Resonance Energy Transfer) homogeneous technologies method (Perkin Elmer). Recombinant ROCK1 (amino acids 1-477) and ROCK2 (amino acids 5-554) proteins were purchased from Carna Biosciences and SignalChem. Compound activities were measured by Envision and IC₅₀s were calculated. The assays were performed in white LUMITRAC™ 200 96 well half-area microplates from Greiner Bio-One. The kinase reaction buffer consisted of 50 mM HEPES pH 7.5, 1 mM EGTA, 10 mM MgCl₂, 2 mM DTT and 0.01% Tween-20. Kinases were incubated with 50 nM (ULight-CREBtide) substrate in the presence of 1 mM (ROCK1) or 1 mM (ROCK2) of ATP. The kinase reaction was carried out for 1 hour before addition of a stopping buffer to a final of 10 mM EDTA and 0.6 nM of LANCE Ultra Europium anti-phospho-CREB (Ser133) antibody (PerkinElmer TRF0200) in LANCE detection buffer. All assay incubations were performed at room temperature, and the microplates were sealed with polyester film during that time. The reaction was incubated for 1 hour, and the signal was read in Envision in TR-FRET mode (excitation at 320 nm and emission at 615/665 nm).

The compounds of Formula (1) exhibited useful pharmacological properties. As used herein, a way to describe potency of inhibitory activity (nM) is a value of inhibitory activity at 50% (IC₅₀). The results are shown in Table xx below, where an IC₅₀ of less than 10 nM is defined as “A,” an IC₅₀ of between 11 nM and 50 nM is defined as “B,” an IC₅₀ of between 51 nM and 500 nM is defined as “C,” and an IC₅₀ of greater than 501 nM is defined as “D.” Table 5 illustrates the inhibition of ROCK1 and ROCK2 by representative compounds of (1).

TABLE 5
Inhibition Activity of ROCK1 and ROCK2 at Km of ATP

| Compound | ROCK1 | ROCK2 |
|---|---|---|
| 1-1 | D | D |
| 1-2 | B | A |
| 1-3 | B | A |
| 1-4 | B | A |
| 1-5 | C | B |
| 1-6 | B | A |
| 1-7 | B | A |
| 1-8 | B | A |
| 1-9 | B | A |
| 1-10 | B | A |
| 1-11 | B | B |
| 1-12 | B | A |
| 1-13 | B | A |
| 1-14 | A | A |
| 1-15 | A | A |
| 1-16 | B | A |
| 1-17 | A | A |
| 1-18 | D | C |
| 1-19 | A | A |
| 1-20 | A | A |
| 1-21 | A | A |
| 1-22 | A | A |
| 1-23 | A | A |
| 1-24 | A | A |
| 1-25 | B | A |
| 1-26 | A | A |
| 1-27 | A | A |
| 1-28 | A | A |
| 1-29 | A | A |
| 1-30 | B | A |
| 1-31 | A | A |
| 1-32 | B | A |
| 1-33 | B | A |
| 1-34 | C | B |
| 1-35 | C | B |
| 1-36 | C | B |
| 1-37 | C | B |
| 1-38 | B | A |
| 1-39 | B | B |
| 1-40 | B | A |
| 1-41 | B | A |
| 1-42 | B | A |
| 1-43 | C | B |
| 1-44 | B | B |
| 1-45 | B | A |
| 1-46 | A | A |
| 1-47 | B | A |
| 1-48 | A | A |
| 1-49 | A | A |
| 1-50 | A | A |
| 1-51 | A | A |
| 1-52 | C | B |
| 1-53 | C | B |
| 1-54 | B | A |
| 1-55 | A | A |
| 1-56 | A | A |
| 1-57 | A | A |
| 1-58 | B | A |
| 1-59 | C | B |
| 1-60 | A | A |
| 2-1 | A | A |
| 2-2 | A | A |
| 2-3 | C | B |
| 2-4 | A | A |
| 2-5 | C | B |
| 2-6 | B | A |
| 2-7 | A | A |
| 2-8 | C | C |
| 2-9 | B | A |
| 2-10 | C | B |
| 2-11 | C | A |
| 2-12 | C | C |
| 2-13 | C | B |
| 2-14 | B | A |
| 2-15 | A | A |
| 2-16 | B | A |
| 2-17 | B | B |
| 2-18 | B | A |
| 2-19 | D | C |
| 2-20 | B | B |
| 2-21 | B | A |
| 2-22 | D | C |
| 2-23 | B | B |
| 2-24 | B | A |
| 2-25 | B | B |
| 2-26 | C | C |
| 2-27 | B | A |
| 2-28 | D | D |
| 2-29 | D | C |
| 2-30 | C | C |
| 2-31 | C | C |
| 2-32 | D | C |
| 2-33 | C | B |
| 2-34 | C | B |
| 2-35 | C | B |
| 3-1 | B | A |
| 3-2 | B | A |
| 3-3 | B | A |
| 3-4 | C | A |

1. A compound of Formula (1), a pharmaceutically acceptable salt of the compound, or a prodrug of the compound or the salt: 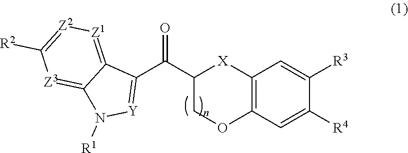
wherein:
X is CH₂ or O;
Y is N or CH;
Z¹, Z² and Z³ each are independently N or CH;
n is 0, 1, 2 or 3;
R¹ is H, C₂-C₆alkyl, or CH₂C₃-C₈cyclocloalkyl wherein the C₂-C₆alkyl or CH₂C₃-C₈cyclocloalkyl is optionally substituted with one or more substituents selected from the group consisting of C₁-C₃alkyl, hydroxyl, CN, and C₁-C₃alkoxy;
R² is heteroaryl or bicyclic heteroaryl, wherein the heteroaryl or bicyclic heteroaryl is optionally substituted with one or more suitable substituents selected from the group consisting of halogen, CN, CHF₂, CF₃, C₁-C₃alkyl, and amino, wherein heteroaryl, bicyclic heteroaryl has 1-3 heteroatoms selected from the group consisting of oxygen and nitrogen; and
R³ and R⁴ are independently H, Cl, F, OH, OCD³, C₁-C₃alkyl, or C₁-C₃alkoxy, wherein the C₁-C₃alkyl or C₁-C₃alkoxy is optionally substituted with one or more suitable substituents, for example, OH or OMe, OEt, OPrⁱ.

2. The compound, salt, or prodrug of claim 1, wherein R² is selected from the following group:

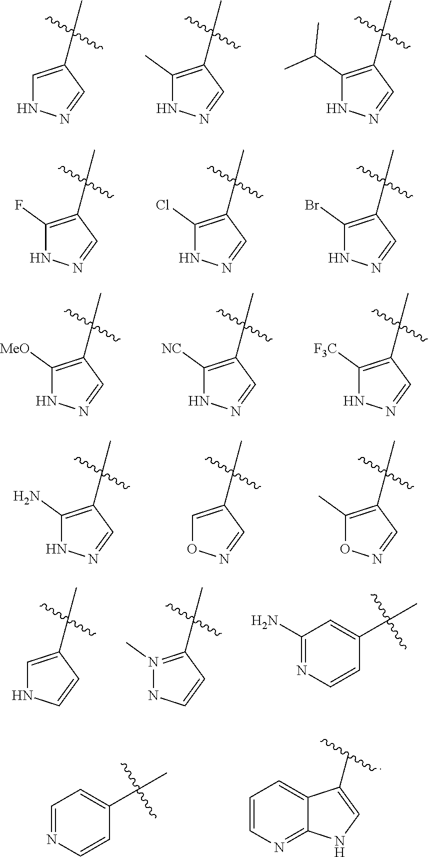

3. The compound, salt, or prodrug of claim 1, wherein R³ and R⁴ are independently H, Cl, F, OH, OMe, OCD³, OEt, OPrⁱ, or C₁-C₃alkyl, wherein the C₁-C₃alkyl is optionally substituted with one or more suitable substituents selected from the group consisting of OH, OMe, OEt, and OPrⁱ.

4. A pharmaceutical composition comprising the compound, salt, or prodrug of claim 1 and an additional component selected from the group consisting of a pharmaceutically acceptable carrier, diluent, excipient, adjuvant, and a combination thereof.

5. A compound represented by the following list, a pharmaceutically acceptable salt of the compound, or a prodrug of the compound or the salt:
(6-Chlorochroman-3-yl)-[1-(2-hydroxyethyl)-6-(3-methoxy-1H-pyrazol-4-yl)pyrrolo[3,2-c]pyridin-3-yl]methanone;
(6-Chloro-2H-chromen-3-yl)-[6-(3-chloro-1H-pyrazol-4-yl)-1-(2-hydroxyethyl)pyrrolo[3,2-c]pyridin-3-yl]methanone;
(6-(3-Chloro-1H-pyrazol-4-yl)-1-(2-hydroxyethyl)-1H-pyrrolo[2,3-b]pyridin-3-yl)(6-fluorochroman-3-yl)methanone;
(6-Chlorochroman-3-yl)-[6-(3-chloro-1H-pyrazol-4-yl)-1-(2-hydroxyethyl)pyrrolo[2,3-b]pyridin-3-yl]methanone;
(6-Chlorochroman-3-yl)-[6-(3-chloro-1H-pyrazol-4-yl)-1-(2-hydroxyethyl)pyrrolo[3,2-b]pyridin-3-yl]methanone;
(6-Chlorochroman-3-yl)-[6-(3-chloro-1H-pyrazol-4-yl)-1-(2-hydroxypropyl)pyrrolo[3,2-c]pyridin-3-yl]methanone
(6-Chlorochroman-3-yl)-[6-(5-chloro-1H-pyrazol-4-yl)-1-[(2S)-2-hydroxypropyl]pyrrolo[3,2-c]pyridin-3-yl]methanone;
(6-Chlorochroman-3-yl)-[6-(3-chloro-1H-pyrazol-4-yl)-1-[(2R)-2-hydroxypropyl]pyrrolo[3,2-c]pyridin-3-yl]methanone;
(6-(5-Chloro-1H-pyrazol-4-yl)-1-((S)-2-hydroxypropyl)-1H-pyrrolo[3,2-c]pyridin-3-yl)((S)-6-chlorochroman-3-yl)methanone;
(6-(5-Chloro-1H-pyrazol-4-yl)-1-((S)-2-hydroxypropyl)-1H-pyrrolo[3,2-c]pyridin-3-yl)((R)-6-chlorochroman-3-yl)methanone;
(6-(5-Chloro-1H-pyrazol-4-yl)-1-((S)-2-hydroxypropyl)-1H-pyrrolo[3,2-c]pyridin-3-yl)((S)-6-chlorochroman-3-yl)methanone methanesulfonic acid;
[6-(3-Chloro-1H-pyrazol-4-yl)-1-(2-hydroxyethyl)pyrrolo[3,2-c]pyridin-3-yl]-(6-fluorochroman-3-yl)methanone;
[6-(3-chloro-1H-pyrazol-4-yl)-1-(2-hydroxypropyl)pyrrolo[3,2-c]pyridin-3-yl]-(6-fluorochroman-3-yl)methanone;
(6-Fluorochroman-3-yl)-[1-(2-hydroxyethyl)-6-(3-methyl-1H-pyrazol-4-yl)pyrrolo[2,3-b]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone;
(6-Chlorochroman-3-yl)-[6-(3-chloro-1H-pyrazol-4-yl)-1-(2-hydroxy-2-methyl-propyl)pyrrolo[3,2-c]pyridin-3-yl]methanone hydrochloride;
[6-(3-Chloro-1H-pyrazol-4-yl)-1-[(2S)-2-hydroxypropyl]pyrrolo[3,2-c]pyridin-3-yl]-(6-fluorochroman-3-yl)methanone;
[[6-(3-Chloro-1H-pyrazol-4-yl)-1-[(2R)-2-hydroxypropyl]pyrrolo[3,2-c]pyridin-3-yl]-(6-fluorochroman-3-yl)methanone;
[6-(3-Chloro-1H-pyrazol-4-yl)-1-(2-hydroxyethyl)pyrrolo[2,3-b]pyridin-3-yl]-(6-methoxychroman-3-yl)methanone;
[6-(3-Chloro-1H-pyrazol-4-yl)-1-(2-hydroxyethyl)pyrrolo[3,2-c]pyridin-3-yl]-(6-methoxychroman-3-yl)methanone;
[6-(5-Fluoro-1H-pyrazol-4-yl)-1-(2-hydroxyethyl)pyrrolo[3,2-c]pyridin-3-yl]-(6-methoxychroman-3-yl)methanone;
[6-(5-Fluoro-1H-pyrazol-4-yl)-1-[(2S)-2-hydroxypropyl]pyrrolo[3,2-c]pyridin-3-yl]-(6-methoxychroman-3-yl)methanone;
[6-(5-Fluoro-1H-pyrazol-4-yl)-1-[(2S)-2-hydroxypropyl]pyrrolo[3,2-c]pyridin-3-yl]-(6-methoxychroman-3-yl)methanone;
[6-(5-Chloro-1H-pyrazol-4-yl)-1-[(2S)-2-hydroxypropyl]pyrrolo[3,2-c]pyridin-3-yl]-(6-methoxychroman-3-yl)methanone;
[6-(5-Fluoro-1H-pyrazol-4-yl)-1-(2-hydroxy-2-methyl-propyl)pyrrolo[3,2-c]pyridin-3-yl]-(6-methoxychroman-3-yl)methanone;
[6-(5-Chloro-1H-pyrazol-4-yl)-1-(2-hydroxy-2-methyl-propyl)pyrrolo[3,2-c]pyridin-3-yl]-(6-methoxychroman-3-yl)methanone;
[(3S)-6-Chlorochroman-3-yl]-[6-(5-fluoro-1H-pyrazol-4-yl)-1-[(2S)-2-hydroxypropyl]pyrrolo[3,2-c]pyridin-3-yl]methanone;
[(3S)-6-Chlorochroman-3-yl]-[6-(5-fluoro-1H-pyrazol-4-yl)-1-(2-hydroxy-2-methyl-propyl)pyrrolo[3,2-c]pyridin-3-yl]methanone;
[(3S)-6-Chlorochroman-3-yl]-[6-(5-chloro-1H-pyrazol-4-yl)-1-(2-hydroxy-2-methyl-propyl)pyrrolo[3,2-c]pyridin-3-yl]methanone;
[(3S)-6-Chlorochroman-3-yl]-[1-[(2S)-2-hydroxypropyl]-6-(5-methoxy-1H-pyrazol-4-yl)pyrrolo[3,2-c]pyridin-3-yl]methanone;
[(3R)-6-Chlorochroman-3-yl]-[1-[(2R)-2-hydroxypropyl]-6-(5-methoxy-1H-pyrazol-4-yl)pyrrolo[3,2-c]pyridin-3-yl]methanone;
(6-Chlorochroman-3-yl)-[6-(5-chloro-1H-pyrazol-4-yl)-1-[(2S)-2-hydroxybutyl]pyrrolo[3,2-c]pyridin-3-yl]methanone;
(6-Chlorochroman-3-yl)-[6-(5-fluoro-1H-pyrazol-4-yl)-1-[(2S)-2-hydroxybutyl]pyrrolo[3,2-c]pyridin-3-yl]methanone;
(6-Chlorochroman-3-yl)-[6-(5-fluoro-1H-pyrazol-4-yl)-1-[(3-hydroxycyclobutyl)methyl]pyrrolo[3,2-c]pyridin-3-yl]methanone;
(6-Chlorochroman-3-yl)-[6-(5-chloro-1H-pyrazol-4-yl)-1-[(3-hydroxycyclobutyl)methyl]pyrrolo[3,2-c]pyridin-3-yl]methanone;
(6-Chlorochroman-3-yl)-[6-(3-chloro-1H-pyrazol-4-yl)-1-(2-hydroxypropyl)indol-3-yl]methanone;
(6-(3-Chloro-1H-pyrazol-4-yl)-1-(2-hydroxypropyl)-1H-indol-3-yl)((S)-6-chlorochroman-3-yl)methanone;
(6-(3-Chloro-1H-pyrazol-4-yl)-1-(2-hydroxypropyl)-1H-indol-3-yl)((S)-6-chlorochroman-3-yl)methanone;
((S)-6-Chlorochroman-3-yl)(1-(2-hydroxypropyl)-6-(3-methoxy-1H-pyrazol-4-yl)-1H-indol-3-yl)methanone;
(6-Chlorochroman-3-yl)-[1-[(2S)-2-hydroxypropyl]-6-(3-methoxy-1H-pyrazol-4-yl)indol-3-yl]methanone;
(6-Chlorochroman-3-yl)-[1-[(2R)-2-hydroxypropyl]-6-(3-methoxy-1H-pyrazol-4-yl)indol-3-yl]methanone;
(6-Chlorochroman-3-yl)-[2-(5-chloro-1H-pyrazol-4-yl)-7-[(2S)-2-hydroxypropyl]pyrrolo[2,3-d]pyrimidin-5-yl]methanone;
(2-(5-Chloro-1H-pyrazol-4-yl)-7-((S)-2-hydroxypropyl)-7H-pyrrolo[2,3-d]pyrimidin-5-yl)((S)-6-chlorochroman-3-yl)methanone;
(6-Chlorochroman-3-yl)-[2-(5-fluoro-1H-pyrazol-4-yl)-7-[(2S)-2-hydroxypropyl]pyrrolo[2,3-d]pyrimidin-5-yl]methanone;
(2-(5-Fluoro-1H-pyrazol-4-yl)-7-((R)-2-hydroxypropyl)-7H-pyrrolo[2,3-d]pyrimidin-5-yl)(6-fluorochroman-3-yl)methanone;
(2-(5-Fluoro-1H-pyrazol-4-yl)-7-((R)-2-hydroxypropyl)-7H-pyrrolo[2,3-d]pyrimidin-5-yl)((S)-6-fluorochroman-3-yl)methanone;
(2-(5-Fluoro-1H-pyrazol-4-yl)-7-((R)-2-hydroxypropyl)-7H-pyrrolo[2,3-d]pyrimidin-5-yl)((S)-6-fluorochroman-3-yl)methanone hydrochloride;
(2-(5-Fluoro-1H-pyrazol-4-yl)-7-((R)-2-hydroxypropyl)-7H-pyrrolo[2,3-d]pyrimidin-5-yl)((S)-6-fluorochroman-3-yl)methanone methanesulfonic acid;
(6-Fluorochroman-3-yl)(7-(2-hydroxy-2-methylpropyl)-2-(5-methoxy-1H-pyrazol-4-yl)-7H-pyrrolo[2,3-d]pyrimidin-5-yl)methanone;
(6-Chlorochroman-3-yl)-[7-[(2S)-2-hydroxypropyl]-2-(5-methoxy-1H-pyrazol-4-yl)pyrrolo[2,3-d]pyrimidin-5-yl]methanone;
(6-Chlorochroman-3-yl)-[2-(5-fluoro-1H-pyrazol-4-yl)-7-(2-hydroxy-2-methyl-propyl)pyrrolo[2,3-d]pyrimidin-5-yl]methanone;
(S)-(6-Chlorochroman-3-yl)(2-(5-fluoro-1H-pyrazol-4-yl)-7-(2-hydroxy-2-methylpropyl)-7H-pyrrolo[2,3-d]pyrimidin-5-yl)methanone;
(S)-(6-Chlorochroman-3-yl)(2-(5-fluoro-1H-pyrazol-4-yl)-7-(2-hydroxy-2-methylpropyl)-7H-pyrrolo[2,3-d]pyrimidin-5-yl)methanone methanesulfonic acid;
(6-Chlorochroman-3-yl)-[2-(5-chloro-1H-pyrazol-4-yl)-7-(2-hydroxy-2-methyl-propyl)pyrrolo[2,3-d]pyrimidin-5-yl]methanone;
(6-Chlorochroman-3-yl)-[7-(2-hydroxy-2-methyl-propyl)-2-(5-methoxy-1H-pyrazol-4-yl)pyrrolo[2,3-d]pyrimidin-5-yl]methanone;
(6-Chlorochroman-3-yl)(1-(2-hydroxyethyl)-6-(5-methoxy-1H-pyrazol-4-yl)-1H-pyrrolo[3,2-c]pyridin-3-yl)methanone hydrochloride;
(6-Chlorochroman-3-yl)(1-(2-hydroxyethyl)-6-(5-methoxy-1H-pyrazol-4-yl)-1H-pyrrolo[3,2-c]pyridin-3-yl)methanone methanesulfonic acid;
(2,3-Dihydrobenzo[b][1,4]dioxin-2-yl)(1-(2-hydroxyethyl)-6-(1H-pyrazol-4-yl)-1H-indol-3-yl)methanone;
Chroman-3-yl-[1-(2-hydroxyethyl)-6-(1H-pyrazol-4-yl)indol-3-yl]methanone;
2,3-Dihydro-1,4-benzodioxin-3-yl-[1-(2-hydroxyethyl)-6-(1H-pyrrolo[2,3-b]pyridin-3-yl)indol-3-yl]methanone;
[6-(3-Chloro-1H-pyrazol-4-yl)-1-(2-hydroxyethyl)indol-3-yl]-(2,3-dihydro-1,4-benzodioxin-3-yl)methanone;
Chroman-3-yl-[1-(2-hydroxyethyl)-6-(1H-pyrrolo[2,3-b]pyridin-3-yl)indol-3-yl]methanone;
[6-(3-Chloro-1H-pyrazol-4-yl)-1-(2-hydroxyethyl)indol-3-yl]-chroman-3-yl-methanone;
(6-Fluorochroman-3-yl)-[1-(2-hydroxyethyl)-6-(1H-pyrazol-4-yl)indol-3-yl]methanone;
[1-(2-Hydroxyethyl)-6-(1H-pyrazol-4-yl)indol-3-yl]-(7-methoxychroman-3-yl)methanone;
(6-Fluoro-2,3-dihydro-1,4-benzodioxin-3-yl)-[1-(2-hydroxyethyl)-6-(1H-pyrazol-4-yl)indol-3-yl]methanone;
(6-Chloro-2,3-dihydro-1,4-benzodioxin-2-yl)-[1-(2-hydroxyethyl)-6-(1H-pyrazol-4-yl)indol-3-yl]methanone;
(6-Fluoro-2,3-dihydro-1,4-benzodioxin-2-yl)-[1-(2-hydroxyethyl)-6-(1H-pyrazol-4-yl)indol-3-yl]methanone;
(6,7-Dichloro-2,3-dihydro-1,4-benzodioxin-3-yl)-[1-(2-hydroxyethyl)-6-(1H-pyrazol-4-yl)indol-3-yl]methanone;
(6-Chloro-2,3-dihydro-1,4-benzodioxin-3-yl)-[1-(2-hydroxyethyl)-6-(1H-pyrazol-4-yl)indol-3-yl]methanone;
[1-(2-Hydroxyethyl)-6-(1H-pyrazol-4-yl)indol-3-yl]-(6-methoxy-2,3-dihydro-1,4-benzodioxin-3-yl)methanone;
[6-(3-Chloro-1H-pyrazol-4-yl)-1-(2-hydroxyethyl)indol-3-yl]-(6-methoxychroman-3-yl)methanone;
(6-Chlorochroman-3-yl)(6-(3-fluoro-1H-pyrazol-4-yl)-1-(2-hydroxyethyl)-1H-indol-3-yl)methanone;
(6-Chlorochroman-3-yl)(1-(2-hydroxyethyl)-6-(3-methoxy-1H-pyrazol-4-yl)-1H-indol-3-yl)methanone;
(S)-(6-Chlorochroman-3-yl)(1-(2-hydroxyethyl)-6-(3-methoxy-1H-pyrazol-4-yl)-1H-indol-3-yl)methanone;
(R)-(6-Chlorochroman-3-yl)(1-(2-hydroxyethyl)-6-(3-methoxy-1H-pyrazol-4-yl)-1H-indol-3-yl)methanone;
(6-Fluorochroman-3-yl)(1-(2-hydroxyethyl)-6-(3-methoxy-1H-pyrazol-4-yl)-1H-indol-3-yl)methanone;
(S)-(6-Fluorochroman-3-yl)(1-(2-hydroxyethyl)-6-(3-methoxy-1H-pyrazol-4-yl)-1H-indol-3-yl)methanone;
(R)-(6-Fluorochroman-3-yl)(1-(2-hydroxyethyl)-6-(3-methoxy-1H-pyrazol-4-yl)-1H-indol-3-yl)methanone;
Chroman-3-yl(1-(2-hydroxyethyl)-6-(1H-pyrazol-4-yl)-1H-indol-3-yl)methanone;
Chroman-3-yl(1-(2-methoxyethyl)-6-(1H-pyrazol-4-yl)-1H-indazol-3-yl)methanone;
(S)-(2,3-Dihydrobenzo[b][1,4]dioxin-2-yl)(1-(2-methoxyethyl)-6-(1H-pyrazol-4-yl)-1H-indazol-3-yl)methanone;
(6-Fluorochroman-3-yl)(1-(2-hydroxyethyl)-6-(3-(trifluoromethyl)-1H-pyrazol-4-yl)-1H-indazol-3-yl)methanone;
(6-Fluorochroman-3-yl)(1-(2-hydroxyethyl)-6-(3-methoxy-1H-pyrazol-4-yl)-1H-indazol-3-yl)methanone;
(6-Fluorochroman-3-yl)(1-(2-hydroxyethyl)-6-(3-isopropyl-1H-pyrazol-4-yl)-1H-indazol-3-yl)methanone;
2-(3-(6-Chlorochromane-3-carbonyl)-6-(3-methoxy-1H-pyrazol-4-yl)-1H-indazol-1-yl)acetonitrile
(2,3-Dihydrobenzofuran-2-yl)(1-(2-hydroxyethyl)-6-(3-methoxy-1H-pyrazol-4-yl)-1H-indazol-3-yl)methanone;
(1-(2-Hydroxyethyl)-6-(3-methoxy-1H-pyrazol-4-yl)-1H-indazol-3-yl)(2-methyl-2,3-dihydrobenzo[b][1,4]dioxin-2-yl)methanone;
Benzofuran-3-yl(1-(2-hydroxyethyl)-6-(3-methoxy-1H-pyrazol-4-yl)-1H-indazol-3-yl)methanone;
(6-Chlorochroman-3-yl)(1-(2-hydroxypropyl)-6-(3-methoxy-1H-pyrazol-4-yl)-1H-indazol-3-yl)methanone;
(6-Chlorochroman-3-yl)(1-((R)-2-hydroxypropyl)-6-(3-methoxy-1H-pyrazol-4-yl)-1H-indazol-3-yl)methanone;
(6-Chlorochroman-3-yl)(1-((S)-2-hydroxypropyl)-6-(3-methoxy-1H-pyrazol-4-yl)-1H-indazol-3-yl)methanone;
(6-Fluorochroman-3-yl)(1-(2-hydroxyethyl)-6-(1H-pyrazol-4-yl)-1H-indazol-3-yl)methanone;
(Chroman-3-yl(1-(2-hydroxyethyl)-6-(1H-pyrazol-4-yl)-1H-indazol-3-yl)methanone;
(7-Fluoro-2,3-dihydrobenzo[b][1,4]dioxin-2-yl)(1-(2-hydroxyethyl)-6-(1H-pyrazol-4-yl)-1H-indazol-3-yl)methanone; and
(7-Chloro-2,3-dihydrobenzo[b][1,4]dioxin-2-yl)(1-(2-hydroxyethyl)-6-(1H-pyrazol-4-yl)-1H-indazol-3-yl)methanone.

6. Use of the compound, salt, or prodrug of claim 1 for preparing a medicament for treating a disease that can be treated by inhibition of a ROCK enzyme.

7. Use of the compound, salt, or prodrug of claim 1 for treating or alleviating a fibrotic disorder, a cardiovascular disease or disorder, an inflammatory disease or disorder, or a proliferative disease or disorder.

8. The use of claim 7, wherein the fibrotic disorder is pulmonary fibrosis selected from the group consisting of cystic and idiopathic pulmonary fibrosis, radiation induced lung injury, liver fibrosis including cirrhosis, cardiac fibrosis including arterial fibrosis, endomyocardial fibrosis, old myocardial infraction, arterial stiffness, atherosclerosis, restenosis, arthrofibrosis, Crohn's disease, myelofibrosis, Peyronie's diseases, nephrogenic systemic fibrosis, progressive massive fibrosis, retroperitoneal cavity fibrosis, schleroderma/systemic sclerosis, mediastinal fibrosis, Keloids and hypertrophic scars, glial scaring, and renal fibrosis.

9. The use of claim 7, wherein the cardiovascular disease or disorder is selected from the group consisting of angina, atherosclerosis, cerebral vasospasm, cerebrovascular contraction, coronary vasospasm, endothelial dysfunction, erectile dysfunction, glaucoma, hypertension, ischemic/reperfusion injury, myocardial hypertrophy, myocardial infarction, peripheral circulation disorder, pre-term labor, Raynaud's Disease, renal disease, and stroke.

10. The use of claim 7, wherein the proliferative disease or disorder is an invasive or metastatic cancer selected from the group consisting of adenocarcinoma, adrenocortical cancer, bladder cancer, bone cancer, brain cancer, breast cancer, cancer of the buccal cavity, cervical cancer, colon cancer, colorectal cancer, endometrial or uterine carcinoma, epidermoid carcinoma, esophageal cancer, eye cancer, follicular carcinoma, gallbladder cancer, gastrointestinal cancer, cancer of the genitourinary tract, glioblastoma, hairy cell carcinoma, head and neck cancer, hepatic carcinoma, hepatocellular cancer, Hodgkin's disease, keratoacarithoma, kidney cancer, large cell carcinoma, cancer of the large intestine, laryngeal cancer, liver cancer, adenocarcinoma of the lung, small-cell lung cancer, squamous carcinoma of the lung, non-small cell lung cancer, melanoma, a myeloproliferative disorder, neuroblastoma, ovarian cancer, papillary carcinoma, pancreatic cancer, cancer of the peritoneum, prostate cancer, rectal cancer, salivary gland carcinoma, sarcoma, squamous cell cancer, small cell carcinoma, cancer of the small intestine, stomach cancer, testicular cancer, thyroid cancer, and vulval cancer.
